(19) United States
(12) Patent Application Publication
(10) Pub. No.: US 2009/0285719 A1
(43) Pub. Date: Nov. 19, 2009

(54) Compressible Transparent Sealing for Open Microplates

(75) Inventors: Kirk M. Hirano, Foster City, CA (US); James C. Nurse, Pleasanton, CA (US); Albert L. Carrillo, Redwood City, CA (US); Mike Y. Lu, San Jose, CA (US); Adrian Fawcett, Pleasanton, CA (US)

Correspondence Address: LIFE TECHNOLOGIES CORPORATION;C/O INTELLEVATE, P.O. BOX 52050, MINNEAPOLIS, MN, 55402, US

(73) Assignee: LIFE TECHNOLOGIES CORPORATION, Carlsbad, CA (US)

(21) Appl. No.: 12/471,300

(22) Filed: May 22, 2009

Related U.S. Application Data
(63) Continuation of application No. 11/768,369, filed on Jun. 26, 2007, now abandoned.
(60) Provisional application No. 60/816,689, filed on Jun. 26, 2006.
(60) Provisional application No. 60/816,814, filed on Jun. 26, 2006.
(60) Provisional application No. 60/816,816, filed on Jun. 26, 2006.
(60) Provisional application No. 60/816,817, filed on Jun. 26, 2006.

(51) Int. Cl. G01N 33/00

(52) U.S. Cl. 422/68.1

(57) ABSTRACT

An apparatus for sealing a microplate, wherein the apparatus comprises a microplate having a first surface and an opposing second surface. A plurality of wells is formed in the first surface of the microplate, wherein each of the plurality of wells is sized to receive an assay therein. A sealing cover is disposed over the microplate adjacent the plurality of wells and is compliant to accommodate variations between the sealing cover and the microplate and/or distribute loads evenly therebetween.

### CROSS-REFERENCE TO RELATED APPLICATIONS

This application is a continuation of application Ser. No. 11/768,369 filed Jun. 26, 2007, which claims the benefit of U.S. Provisional Application No. 60/816,689 filed on Jun. 26, 2006, U.S. Provisional Application No. 60/816,814 filed on Jun. 26, 2006, U.S. Provisional Application No. 60/816,816 filed on Jun. 26, 2006, and U.S. Provisional Application No. 60/816,817 filed on Jun. 26, 2006. The disclosures of the above applications are incorporated herein by reference.

All literature and similar materials cited in this application, including but not limited to, patents, patent applications, articles, books, treatises, and internet web pages, regardless of the format of such literature and similar materials, are expressly incorporated by reference in their entirety for any purpose. In the event that one or more of the incorporated literature and similar materials differs from or contradicts this application, including but not limited to defined terms, term usage, described techniques, or the like, this application controls.

### INTRODUCTION

Currently, genomic analysis, including that of the estimated 30,000 human genes is a major focus of basic and applied biochemical and pharmaceutical research. Such analysis may aid in developing diagnostics, medicines, and therapies for a wide variety of disorders. However, the complexity of the human genome and the interrelated functions of genes often make this task difficult. There is a continuing need for methods and apparatus to aid in such analysis.

### DRAWINGS

The skilled artisan will understand that the drawings, described herein, are for illustration purposes only. The drawings are not intended to limit the scope of the present teachings in any way.

FIG. 1 is a perspective view illustrating a high-density sequence detection system according to some embodiments of the present teachings;

FIG. 2 is a top perspective view illustrating a microplate in accordance with some embodiments;

FIG. 3 is a top perspective view illustrating a microplate in accordance with some embodiments;

FIG. 4 is an enlarged perspective view illustrating a microplate in accordance with some embodiments comprising a plurality of wells comprising a circular rim portion;

FIG. 5 is an enlarged perspective view illustrating a microplate in accordance with some embodiments comprising a plurality of wells comprising a square-shaped rim portion;

FIG. 6 is a cross-sectional view illustrating a well comprising a pressure relief bore according to some embodiments;

FIG. 7 is a cross-sectional view illustrating the well of FIG. 6 wherein the pressure relief bore is partially filled;

FIG. 8 is a cross-sectional view illustrating a well comprising an offset pressure relief bore according to some embodiments, being filled by a spotting device;

FIG. 9 is a cross-sectional view illustrating the well of FIG. 8 being filled by a micro-piezo dispenser;

FIG. 10 is a cross-sectional view illustrating a microplate employing a plurality of apertures, a backing sheet, and a sealing cover according to some embodiments;

FIG. 11 is a top view illustrating a microplate in accordance with some embodiments comprising one or more grooves;

FIG. 12 is an enlarged top view illustrating a corner of the microplate illustrated in FIG. 11;

FIG. 13 is a cross-sectional view of the microplate of FIG. 12 taken along Line 13-13;

FIG. 14 is an enlarged top view illustrating a corner of a microplate according to some embodiments;

FIG. 15 is a cross-sectional view of the microplate of FIG. 14 taken along Line 15-15;

FIG. 16 is a top view illustrating a microplate in accordance with some embodiments comprising at least one thermally isolated portion;

FIG. 17 is a side view illustrating the microplate of FIG. 16;

FIG. 18 is a bottom view illustrating the microplate of FIG. 16;

FIG. 19 is an enlarged cross-sectional view illustrating the microplate of FIG. 16 taken along Line 19-19;

FIG. 20 is an exploded perspective view illustrating a filling apparatus according to some embodiments;

FIG. 21 is a cross-sectional perspective view of the filling apparatus of FIG. 20;

FIG. 22(a) is a cross-sectional perspective view of a filling apparatus according to some embodiments;

FIG. 22(b) is a cross-sectional view of a portion of a filling apparatus comprising a plurality of staging capillaries, microfluidic channels, and ramp features according to some embodiments;

FIG. 23 is a schematic cross-sectional view illustrating exaggerated variations between the sealing cover and the microplate, and the microplate and the thermocycler block;

FIG. 24 is a schematic cross-sectional view illustrating a heated window contacting the sealing cover to eliminate the variations shown in FIG. 23;

FIG. 25 is an enlarged perspective view illustrating a microplate in accordance with some embodiments comprising a compressible rim about each well;

FIG. 26 is a cross-sectional view illustrating a well of a microplate according to some embodiments;

FIG. 27 is a cross-sectional view illustrating a well of an inverted microplate according to some embodiments;

FIG. 28 is a cross-sectional view illustrating a sealing cover according to some embodiments;

FIG. 29 is a cross-sectional view illustrating a hot roller apparatus that can be used to seal a sealing cover to a microplate according to some embodiments;

FIG. 30 is a cross-sectional view illustrating a pressure clamp system according to some embodiments comprising an inflatable transparent bag;

FIG. 31 is a cross-sectional view illustrating a pressure clamp system according to some embodiments comprising a moveable transparent window;

FIG. 32 is a cross-sectional view illustrating a pressure clamp system according to some embodiments comprising an inverted microplate;

FIG. 33 is a cross-sectional view illustrating a pressure clamp system according to some embodiments comprising a plurality of apertures in a microplate;

FIG. 34 is a cross-sectional view illustrating a pressure clamp system according to some embodiments comprising a pressure chamber engaging a sealing cover;

FIG. 35 is a cross-sectional view illustrating a pressure clamp system according to some embodiments comprising a pressure chamber used together with an inverted microplate;

FIG. 36 is a cross-sectional view illustrating a pressure clamp system according to some embodiments comprising a pressure chamber used together with a microplate comprising a plurality of apertures;

FIG. 37 is a cross-sectional view illustrating a pressure clamp system according to some embodiments comprising a pressure chamber engaging a thermocycler block;

FIG. 38 is a cross-sectional view illustrating a pressure clamp system according to some embodiments comprising a vacuum assist system;

FIG. 39 is a cross-sectional view illustrating a pressure clamp system according to some embodiments comprising a pressure chamber engaging a thermocycler block and a microplate;

FIG. 40 is a cross-sectional view illustrating a pressure clamp system according to some embodiments comprising a pressure chamber and a relief port;

FIG. 41 is an exploded cross-sectional view illustrating a pressure clamp system according to some embodiments comprising a heatable transparent window;

FIG. 42 is a top perspective view illustrating an upright configuration, according to some embodiments, of a thermocycler system, an excitation system, a detection system, and a microplate;

FIG. 43 is a side view illustrating the upright configuration of the thermocycler system, the excitation system, the detection system, and the microplate of FIG. 42;

FIG. 44 is a perspective view illustrating an inverted configuration, according to some embodiments, of a thermocycler system, an excitation system, a detection system, and a microplate;

FIG. 45 is an enlarged perspective view illustrating an excitation system according to some embodiments comprising a plurality of LED excitation sources;

FIG. 46 is an enlarged perspective view illustrating an excitation system according to some embodiments comprising a plurality of LED excitation sources;

FIG. 47 is a side view illustrating the inverted configuration of the thermocycler system, the excitation system, the detection system, and the microplate of FIG. 44;

FIG. 48 is a perspective view illustrating an inverted configuration, according to some embodiments, of a thermocycler system, an excitation system comprising individually mirrored excitation sources, a detection system, and a microplate;

FIG. 49 is an enlarged perspective view illustrating the excitation system comprising individually mirrored excitation sources of FIG. 48;

FIG. 50 is a graph exemplifying vignetting and shadowing relative to excitation source position;

FIG. 51 is a graph exemplifying vignetting and shadowing and an illumination profile according to some embodiments;

FIG. 52 is a schematic view illustrating an excitation source comprising a lens according to some embodiments;

FIG. 53 is a schematic view illustrating an excitation source comprising a concave mirror according to some embodiments;

FIG. 54 is a schematic view illustrating an excitation source comprising a concave mirror and a lens according to some embodiments;

FIG. 55 is a schematic view illustrating multiple excitation sources focused to a point on a microplate according to some embodiments;

FIG. 56 is a schematic view illustrating multiple excitation sources focused to multiple points to achieve a desired irradiance profile according to some embodiments;

FIG. 57 is a flow chart illustrating a manufacturing procedure of preloaded microplates according to some embodiments;

FIG. 58 is a cross-sectional view illustrating a sealing cover according to some embodiments;

FIG. 59 is a perspective view illustrating a sealing cover roll according to some embodiments;

FIG. 60(a) is a schematic cross sectional view illustrating a heated pressure clamp according to some embodiments;

FIG. 60(b) is a schematic cross sectional view illustrating a heated cover design according to some embodiments;

FIG. 60(c) is a schematic cross sectional view illustrating a heated cover design according to some embodiments;

FIG. 60(d) is a schematic cross sectional view illustrating a heated cover design according to some embodiments;

FIG. 60(e) is a schematic cross sectional view illustrating a heated cover design according to some embodiments;

FIG. 60(f) is a schematic cross sectional view illustrating a heated cover design according to some embodiments;

FIG. 61 is an exploded perspective view illustrating a heated pressure clamp according to some embodiments, with portions removed for clarity;

FIG. 62 is a perspective view illustrating a heated pressure clamp according to some embodiments, with portions removed for clarity;

FIG. 63 is a schematic cross sectional view illustrating a heated pressure clamp according to some embodiments with a transparent window in contact with a microplate;

FIG. 64 is a schematic cross sectional view illustrating a heated pressure clamp according to some embodiments;

FIG. 65 is a schematic cross sectional view illustrating a heated pressure clamp according to some embodiments;

FIG. 66 is a schematic cross sectional view illustrating a heated pressure clamp according to some embodiments prior to engagement with a microplate;

FIG. 67 is a schematic cross sectional view illustrating a heated pressure clamp according to some embodiments following engagement with a microplate;

FIG. 68 is a perspective view of a thin film heater;

FIG. 69 is a perspective view of a thin film heater;

FIG. 70 is a schematic cross sectional view illustrating a heated pressure clamp according to some embodiments employing a thin film heater;

FIG. 71 is a schematic cross sectional view illustrating a heated pressure clamp according to some embodiments employing a heated plate;

FIG. 72 is a schematic cross sectional view illustrating a heated pressure clamp according to some embodiments employing a fine wire heater;

FIG. 73 is an enlarged perspective view illustrating a fine wire heater circuit;

FIG. 74 is a schematic cross sectional view illustrating a heated pressure clamp according to some embodiments employing a hot pressure chamber;

FIG. 75 is a schematic cross sectional view illustrating a heated pressure clamp according to some embodiments employing a convective chamber;

FIG. 76 is a schematic cross sectional view illustrating a heated pressure clamp according to some embodiments employing an induction window heater;

FIG. 77 is a schematic cross sectional view illustrating a heated pressure clamp according to some embodiments employing a hot air chamber;

FIGS. 78(a)-(b) are thermal modeling images illustrating the heat distribution of a heated transparent window spaced apart from a microplate defining a gap therebetween during a cold cycle;

FIG. 79 is thermal modeling image illustrating the heat distribution of a heated transparent window spaced apart from a microplate defining a gap therebetween during a hot cycle;

FIG. 80 is a graph illustrating a thermal profile of a heated transparent window spaced apart from a microplate defining a gap therebetween during both a heating cycle and a cooling cycle;

FIG. 81 is an exploded perspective view illustrating a clamp adapter;

FIG. 82 is a side view illustrating a clamp adapter;

FIG. 83 is a perspective view illustrating a clamp adapter;

FIG. 84 is an exploded view illustrating an inverted configuration of a pressure chamber according to some embodiments;

FIG. 85 is a cross-sectional view illustrating section A-A of the pressure chamber of FIG. 84 in combination with a thermocycler system according to some embodiments;

FIG. 86 is a side view illustrating a clamp mechanism in a locked condition according to some embodiments;

FIG. 87 is a side view illustrating a clamp mechanism in an unlocked condition according to some embodiments;

FIG. 88 is a bottom perspective view illustrating a clamp mechanism in a locked condition according to some embodiments;

FIG. 89 is a pneumatic diagram illustrating a pneumatic system for a pressure chamber and a clamp mechanism according to some embodiments;

FIG. 90 is a perspective view illustrating the pneumatic system of FIG. 89 according to some embodiments;

FIG. 91 is a flow diagram illustrating a method of clamping a chamber to a thermocycler system according to some embodiments;

FIG. 92 is a flow diagram illustrating a method of performing a leak test on a chamber according to some embodiments;

FIG. 93 is a flow diagram illustrating a method of unclamping a chamber from a thermocycler system according to some embodiments;

FIG. 94 is a cross-sectional view illustrating an adjustable lens and camera mount according to some embodiments;

FIG. 95 is a schematic cross-sectional view illustrating a transparent window having a diamond thin film;

FIG. 96 is a schematic cross-sectional view illustrating a transparent window having a diamond thin film and a heating device;

FIG. 97 is a plan view illustrating a transparent window having a diamond thin film with a resistive path formed in parallel therein;

FIG. 98 is a plan view illustrating a transparent window having a diamond thin film with a resistive path formed in series therein; and

FIG. 99 is a schematic cross-sectional view illustrating a transparent window with a first diamond thin film having a resistive path and a second diamond thin film disposed over the first diamond thin film.

### DESCRIPTION OF SOME EMBODIMENTS

The following description of some embodiments is merely exemplary in nature and is in no way intended to limit the present teachings, applications, or uses. Although the present teachings will be discussed in some embodiments as relating to polynucleotide amplification, such as PCR, such discussion should not be regarded as limiting the present teaching to only such applications.

The section headings and sub-headings used herein are for general organizational purposes only and are not to be construed as limiting the subject matter described in any way.

### High-Density Sequence Detection System

In some embodiments, a high density sequence detection system comprises one or more components useful in an analytical method or chemical reaction, such as the analysis of biological and other materials containing polynucleotides. Such systems are, in some embodiments, useful in the analysis of assays, as further described below. High density sequence detection systems, in some embodiments, comprise an excitation system and a detection system which can be useful for analytical methods involving the generation and/or detection of electromagnetic radiation (e.g., visible, ultraviolet or infrared light) generated during analytical procedures. In some embodiments, such procedures include those comprising the use of fluorescent or other materials that absorb and/or emit light or other radiation under conditions that allow quantitative and/or qualitative analysis of a material (e.g., assays among those described herein). In some embodiments useful for polynucleotide amplification and/or detection, a high density sequence detection system can further comprise a thermocycler. In some embodiments, a high density sequence system can further comprise microplate and components for, e.g., filling and handling the microplate, such as a pressure clamp system. It will be understood that, although high density sequence detection systems are described herein with respect to specific microplates, assays and other embodiments, such systems and components thereof are useful with a variety of analytical platforms, equipment, and procedures.

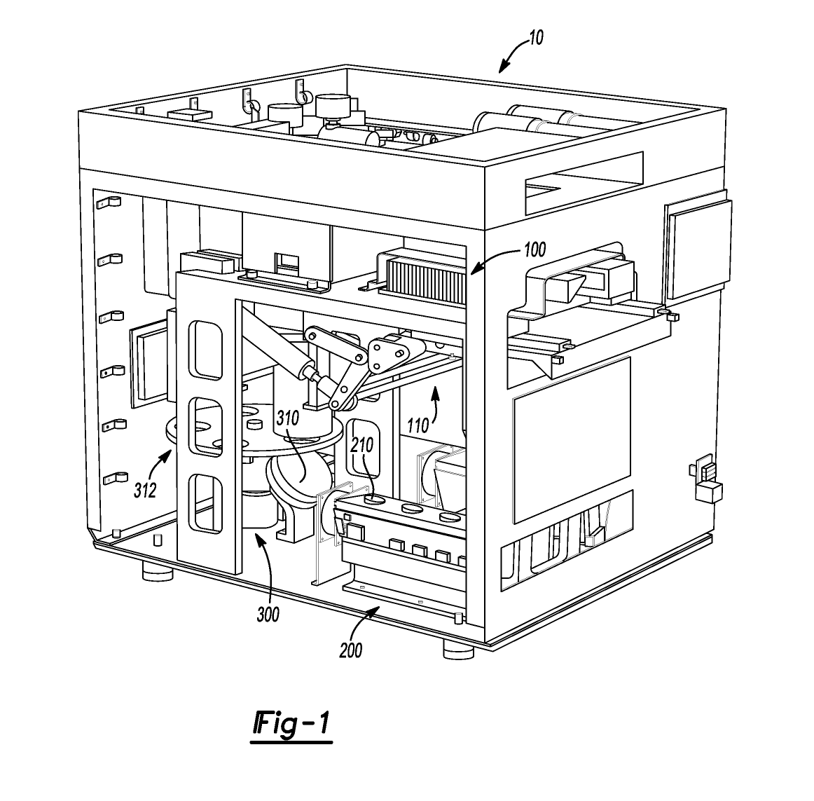

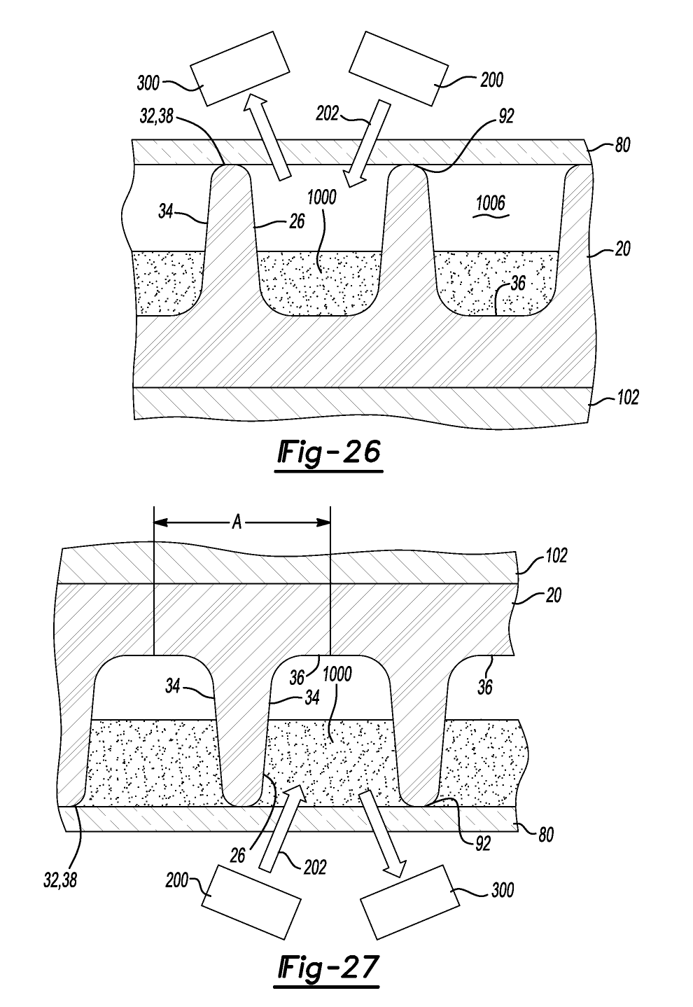

Referring to FIG. 1, a high-density sequence detection system 10 is illustrated in accordance with some embodiments of the present teachings. In some embodiments, high-density sequence detection system 10 comprises a microplate 20 containing an assay 1000 (see FIGS. 26 and 27), a thermocycler system 100, a pressure clamp system 110, an excitation system 200, and a detection system 300 disposed in a housing 1008.

In some embodiments, assay 1000 can comprise any material that is useful in, the subject of, a precursor to, or a product of, an analytical method or chemical reaction. In some embodiments for amplification and/or detection of polynucleotides, assay 1000 comprises one or more reagents (such as PCR master mix, as described further herein); an analyte (such as a biological sample comprising DNA, a DNA fragment, cDNA, RNA, or any other nucleic acid sequence), one or more primers, one or more primer sets, one or more detection probes; components thereof; and combinations thereof. In some embodiments, assay 1000 comprises a homogenous solution of a DNA sample, at least one primer set, at least one detection probe, a polymerase, and a buffer, as used in a homogenous assay (described further herein). In some embodiments, assay 1000 can comprise an aqueous solution of at least one analyte, at least one primer set, at least one detection probe, and a polymerase. In some embodiments, assay 1000 can be an aqueous homogenous solution. In some embodiments, assay 1000 can comprise at least one of a plurality of different detection probes and/or primer sets to perform multiplex PCR, which can be useful, for example, when analyzing a whole genome (e.g., 20,000 to 30,000 genes, or more) or other large numbers of genes or sets of genes.

### Microplate

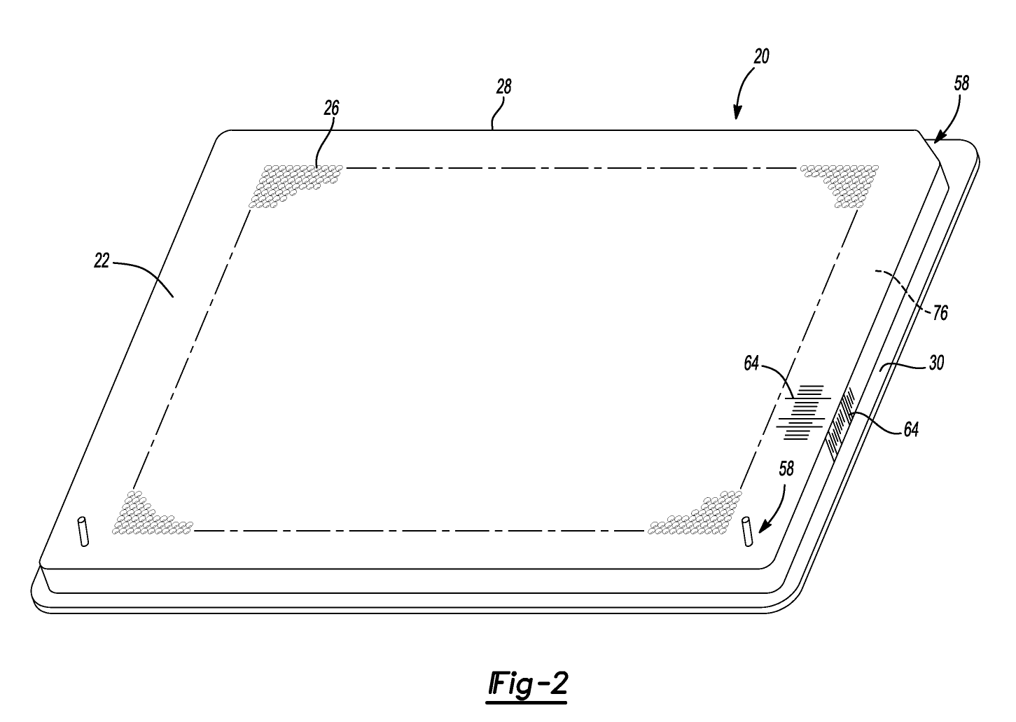

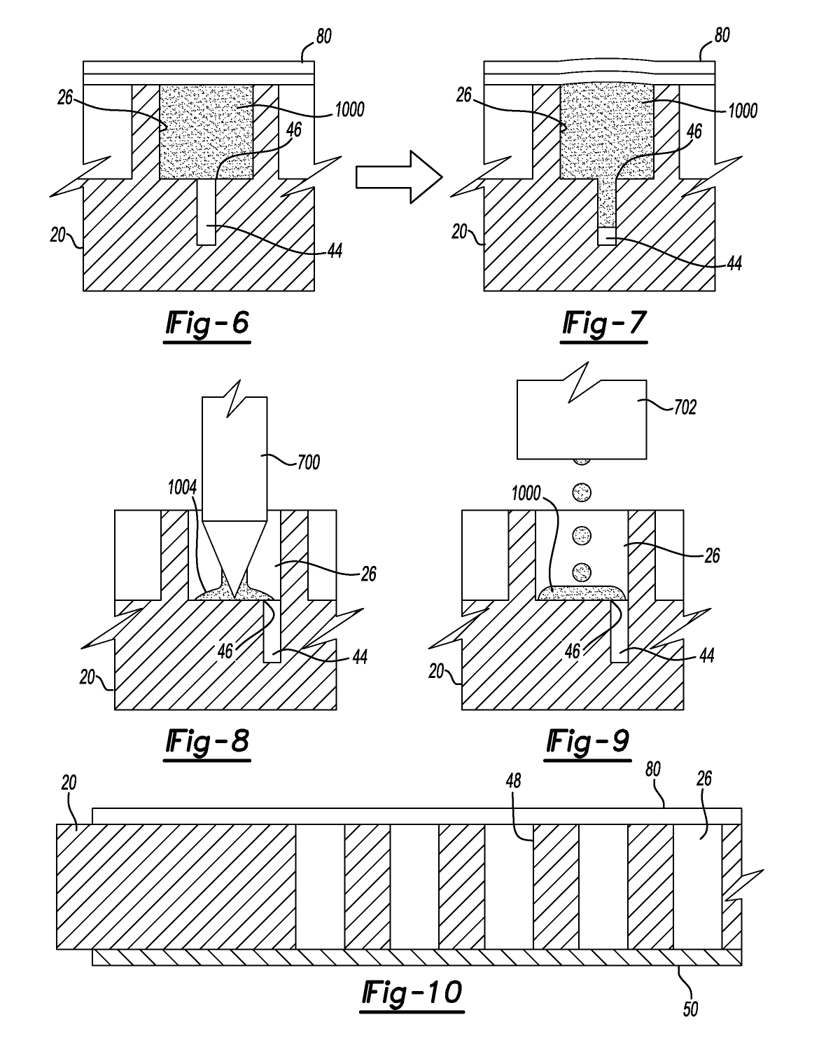

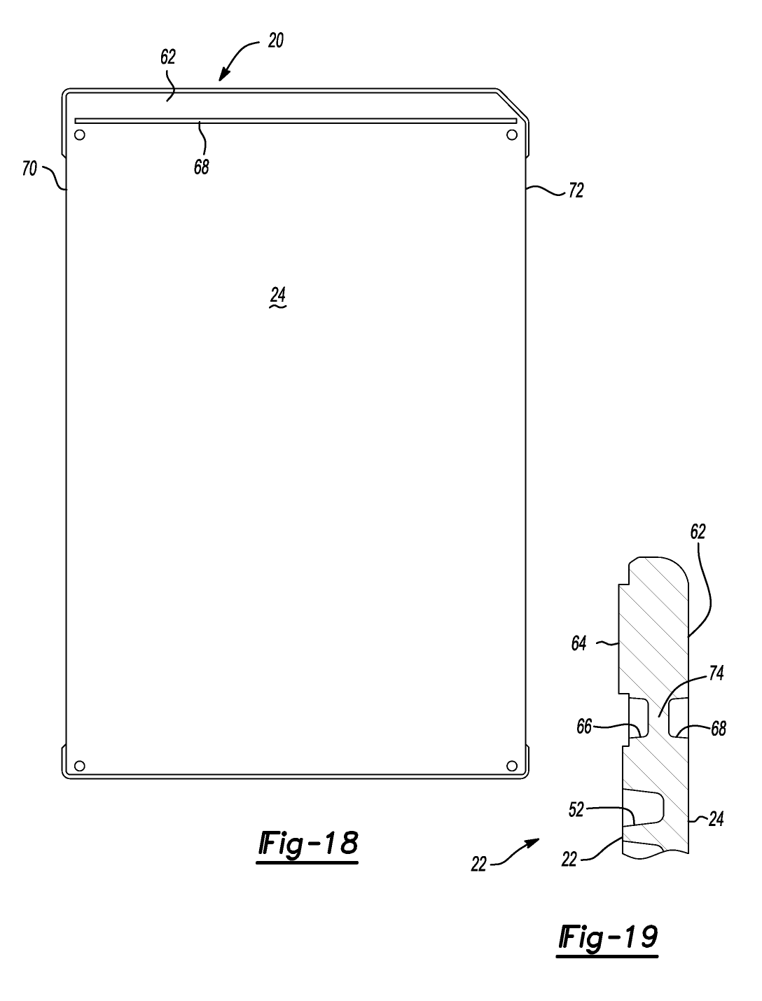

In some embodiments, a microplate comprises a substrate useful in the performance of an analytical method or chemical reaction. In some embodiments, the microplate is substantially planar, having substantially planar upper and lower surfaces, wherein the dimensions of the planar surfaces in the x- and y-dimensions are substantially greater than the thickness of the substrate in the z-direction. In some embodiments, a microplate can comprise one or more material retention regions or reaction chambers, configured to hold or support a material (e.g., an assay, as discussed below, or other solid or liquid) at one or more locations on or in the microplate. In some embodiments, such material retention regions can be wells, through-holes, reaction spots or pads, and the like. In some embodiments, such as shown in FIGS. 2-19, material retention regions comprise wells, as at 26. In some embodiments, such wells can comprise a feature on or in the surface of the microplate wherein assay 1000 is contained at least in part by physical separation from adjacent features. Such well features can include, in some embodiments, depressions, indentations, ridges, and combinations thereof, in regular or irregular shapes. In some embodiments a microplate is single-use, wherein it is filled or otherwise used with a single assay for a single experiment or set of experiments, and is thereafter discarded. In some embodiments, a microplate is multiple-use, wherein it can be operable for use in a plurality of experiments or sets of experiments.

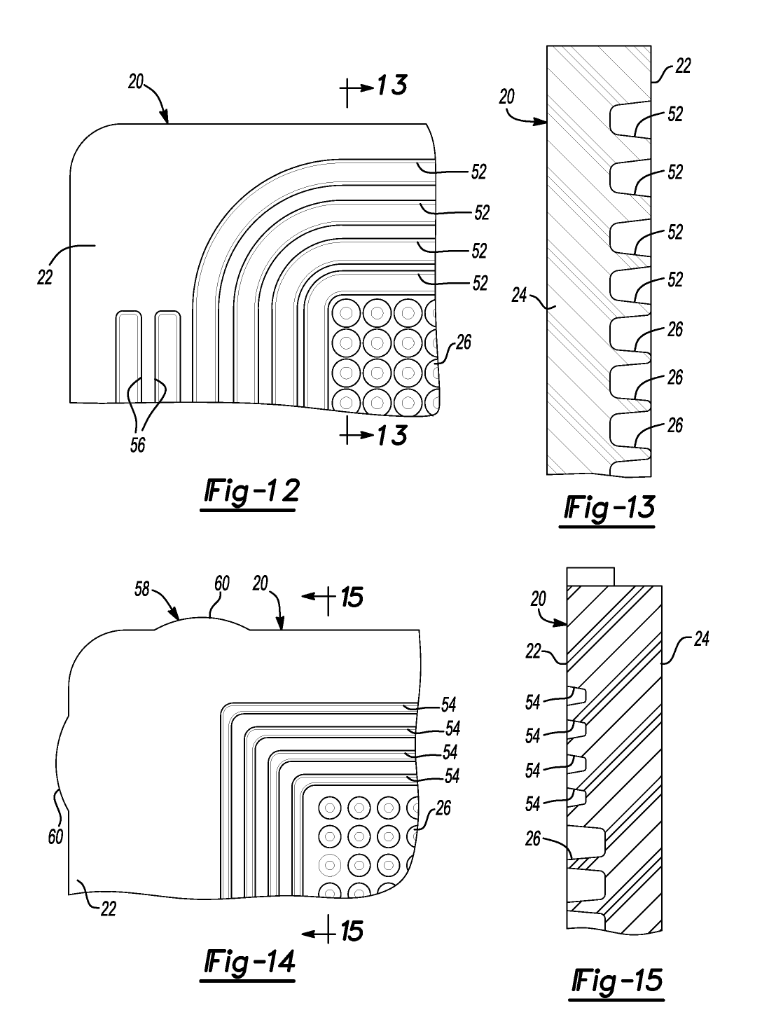

Referring now to FIGS. 2-19, in some embodiments, microplate 20 comprises a substantially planar construction having a first surface 22 and an opposing second surface 24 (see FIG. 12-19). First surface 22 comprises a plurality of wells 26 disposed therein or thereon. The overall positioning of the plurality of wells 26 can be referred to as a well array. Each of the plurality of wells 26 is sized to receive assay 1000 (FIGS. 26 and 27). As illustrated in FIGS. 26 and 27, assay 1000 is disposed in at least one of the plurality of wells 26 and sealing cover 80 (FIG. 26) is disposed thereon (as will be discussed herein). In some embodiments, one or more of the plurality of wells 26 may not be completely filled with assay 1000, thereby defining a headspace 1006 (FIG. 26), which can define an air gap or other gas gap.

In some embodiments, the material retention regions of microplate 20 can comprise a plurality of reaction spots on the surface of the microplate. In such embodiments, a reaction spot can be an area on the microplate which localizes, at least in part by non-physical means, assay 1000. In such embodiments, assay 1000 can be localized in sufficient quantity, and isolation from adjacent areas on the microplate, so as to facilitate an analytical or chemical reaction (e.g., amplification of one or more target DNA) in the material retention region. Such localization can be accomplished by physical and chemical modalities, including, for example, physical containment of reagents in one dimension and chemical containment in one or more other dimensions.

### Microplate Footprint

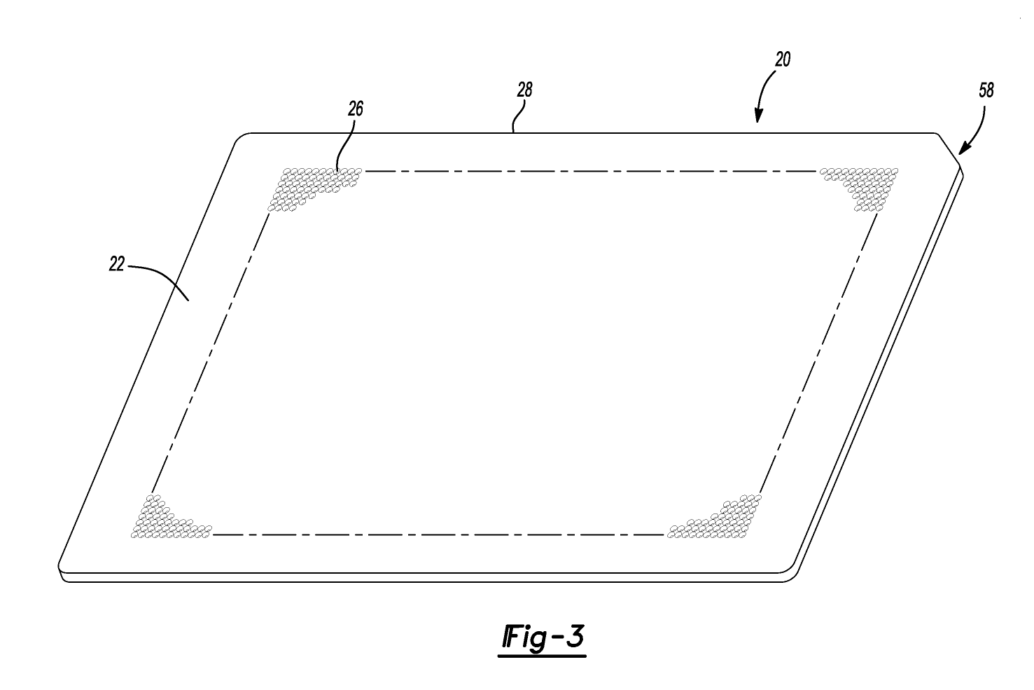

With reference to FIGS. 2-19, microplate 20 generally comprises a main body or substrate 28. In some embodiments, main body 28 is substantially planar. In some embodiments, microplate 20 comprises an optional skirt or flange portion 30 disposed about a periphery of main body 28 (see FIG. 2). Skirt portion 30 can form a lip around main body 28 and can vary in height. Skirt portion 30 can facilitate alignment of microplate 20 on thermocycler block 102. Additionally, skirt portion 30 can provide additional rigidity to microplate 20 such that during handling, filling, testing, and the like, microplate 20 remains rigid, thereby ensuring assay 1000, or any other components, disposed in each of the plurality of wells 26 does not contaminate adjacent wells. However, in some embodiments, microplate 20 can employ a skirtless design (see FIGS. 3-5) depending upon user preference.

In some embodiments, microplate 20 can be from about 50 to about 200 mm in width, and from about 50 to about 200 mm in length. In some embodiments, microplate 20 can be from about 50 to about 100 mm in width, and from about 100 to about 150 mm in length. In some embodiments, microplate 20 can be about 72 mm wide and about 120 mm long.

In order to facilitate use with existing equipment, robotic implements, and instrumentation, the footprint dimensions of main body 28 and/or skirt portion 30 of microplate 20, in some embodiments, can conform to standards specified by the Society of Biomolecular Screening (SBS) and the American National Standards Institute (ANSI), published January 2004 (ANSI/SBS 3-2004). In some embodiments, the footprint dimensions of main body 28 and/or skirt portion 30 of microplate 20 are about 127.76 mm (5.0299 inches) in length and about 85.48 mm (3.3654 inches) in width. In some embodiments, the outside corners of microplate 20 comprise a corner radius of about 3.18 mm (0.1252 inches). In some embodiments, microplate 20 comprises a thickness of about 0.5 mm to about 3.0 mm. In some embodiments, microplate 20 comprises a thickness of about 1.25 mm. In some embodiments, microplate 20 comprises a thickness of about 2.25 mm. One skilled in the art will recognize that microplate 20 and skirt portion 30 can be formed in dimensions other than those specified herein.

### Plurality of Material Retention Regions

The density of material retention regions (i.e., number of material retention regions per unit surface area of microplate) and the size and volume of material retention regions can vary depending on the desired application and such factors as, for example, the species of the organism for which the methods of the present teachings may be employed. In some embodiments, the density of material retention regions can be from about 10 to about 1000 regions/cm², or from about 50 to about 100 regions/cm², for example about 79 regions/cm². In some embodiments, the density of material retention regions can be from about 150 to about 170 regions/cm². In some embodiments, the density of material retention regions can be from about 480 to about 500 regions/cm².

In some embodiments, the pitch of material retention regions on microplate 20 can be from about 50 to about 10000 μm, or from about 50 to about 1500 μm, or from about 450 to 550 μm. In some embodiments, the pitch of material retention regions on microplate 20 can be from about 50 to about 1000 μm, or from about 400 to 500 μm. In some embodiments, the pitch can be from about 1000 to 1200 μm. In some embodiments, the distance between the material retention regions (the thickness of the wall between chambers) can be from about 50 to about 200 μm, or from about 100 to about 200 μm, for example, about 150 μm.

In some embodiments, the total number of material retention regions on the microplate can be from about 5000 to about 100,000, or from about 5000 to about 50,000, or from about 5000 to about 10,000. In some embodiments, the microplate can comprise from about 10,000 to about 15,000 material retention regions. In some embodiments, the microplate can comprise from about 25,000 to about 35,000 material retention regions.

In order to increase throughput of genotyping, gene expression, and other assays, in some embodiments, microplate 20 comprises an increased quantity of the plurality of wells 26 beyond that employed in prior conventional microplates. In some embodiments, microplate 20 comprises 6,144 wells. According to the present teachings, microplate 20 can comprise, but is not limited to, any of the array configurations of wells described in Table 1.

TABLE 1

| Total Number of Wells | Rows × Columns | Approximate Well Area |
|---|---|---|
| 96 | 8 × 12 | 9 × 9 mm |
| 384 | 16 × 24 | 4.5 × 4.5 mm |
| 1536 | 32 × 48 | 2.25 × 2.25 mm |
| 3456 | 48 × 72 | 1.5 × 1.5 mm |
| 6144 | 64 × 96 | 1.125 × 1.125 mm |
| 13824 | 96 × 144 | 0.75 × .075 mm |
| 24576 | 128 × 192 | 0.5625 × 0.5625 mm |
| 55296 | 192 × 288 | 0.375 × 0.375 mm |
| 768 | 24 × 32 | 3 × 3 mm |
| 1024 | 32 × 32 | 2.25 × 3 mm |
| 1600 | 40 × 40 | 1.8 × 2.7 mm |
| 1280 | 32 × 40 | 2.25 × 2.7 mm |
| 1792 | 32 × 56 | 2.25 × 1.714 mm |
| 2240 | 40 × 56 | 1.8 × 1.714 mm |
| 864 | 24 × 36 | 3 × 3 mm |
| 4704 | 56 × 84 | 1.257 × 1.257 mm |
| 7776 | 72 × 108 | 1 × 1 mm |
| 9600 | 80 × 120 | 0.9 × .09 mm |
| 11616 | 88 × 132 | 0.818 × 0.818 mm |
| 16224 | 104 × 156 | 0.692 × 0.692 mm |
| 18816 | 112 × 168 | 0.643 × 0.643 mm |
| 21600 | 120 × 180 | 0.6 × 0.6 mm |
| 27744 | 136 × 204 | 0.529 × 0.529 mm |
| 31104 | 144 × 216 | 0.5 × 0.5 mm |
| 34656 | 152 × 228 | 0.474 × 0.474 mm |
| 38400 | 160 × 240 | 0.45 × 0.45 mm |
| 42336 | 168 × 252 | 0.429 × 0.429 mm |
| 46464 | 176 × 264 | 0.409 × 0.409 mm |
| 50784 | 184 × 256 | 0.391 × 0.391 mm |

### Material Retention Region Size and Shape

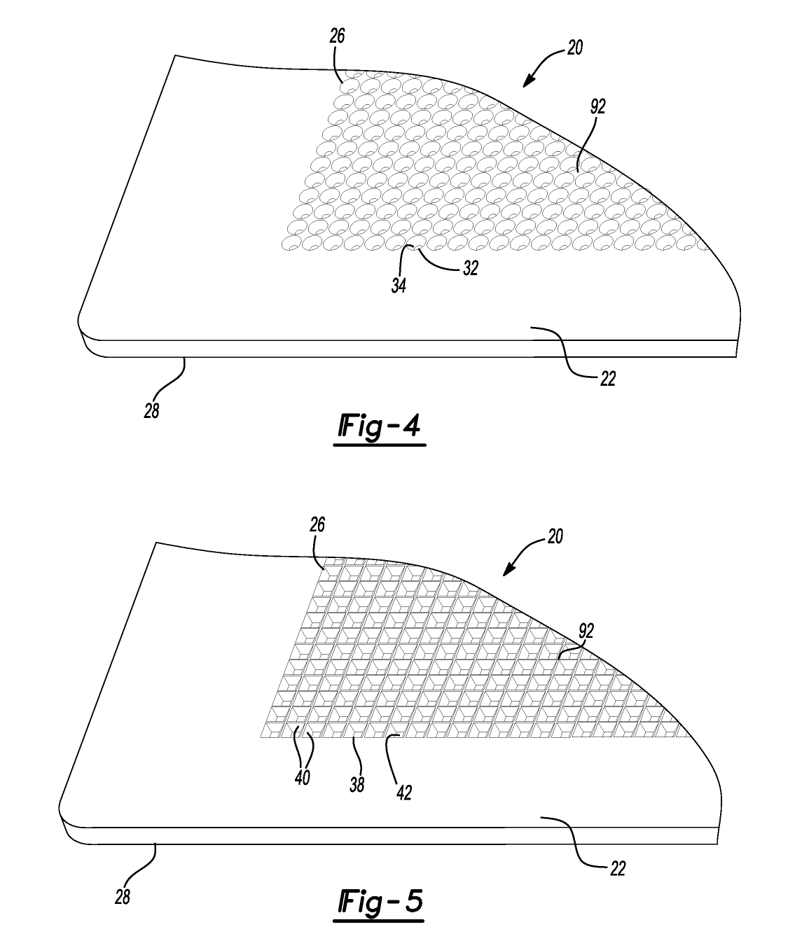

According to some embodiments, as illustrated in FIGS. 4 and 5, each of the plurality of material retention regions (e.g., wells 26) can be substantially equivalent in size. The plurality of wells 26 can have any cross-sectional shape. In some embodiments, as illustrated in FIGS. 4, 26, and 27, each of the plurality of wells 26 comprises a generally circular rim portion 32 (FIG. 4) with a downwardly-extending, generally-continuous sidewall 34 that terminate at a bottom wall 36 interconnected to sidewall 34 with a radius. A draft angle of sidewall 34 can be used in some embodiments. In some embodiments, the draft angle provides benefits including increased ease of manufacturing and minimizing shadowing (as discussed herein). The particular draft angle is determined, at least in part, by the manufacturing method and the size of each of the plurality of wells 26. In some embodiments, circular rim portion 32 can be about 1.0 mm in diameter, the depth of each of the plurality of wells 26 can be about 0.9 mm, the draft angle of sidewall 34 can be about 1° to 5° or greater and each of the plurality of wells 26 can have a center-to-center distance of about 1.125 mm. In some embodiments, the volume of each of the plurality of wells 26 can be about 500 nanoliters.

According to some embodiments, as illustrated in FIG. 5, each of the plurality of wells 26 comprises a generally square-shaped rim portion 38 with downwardly-extending sidewalls 40 that terminate at a bottom wall 42. A draft angle of sidewalls 40 can be used. Again, the particular draft angle is determined, at least in part, by the manufacturing method and the size of each of the plurality of wells 26. In some embodiments of wells 26 of FIG. 5, generally square-shaped rim portion 38 can have a side dimension of about 1.0 mm in length, a depth of about 0.9 mm, a draft angle of about 1° to 5° or greater, and a center-to-center distance of about 1.125 mm, generally indicated at A (see FIG. 27). In some embodiments, the volume of each of the plurality of wells 26 of FIG. 5 can be about 500 nanoliters. In some embodiments, the spacing between adjacent wells 26, as measured at the top of a wall dividing the wells, is less than about 0.5 m. In some embodiments, this spacing between adjacent wells 26 is about 0.25 mm.

In some embodiments, and in some configurations, the plurality of wells 26 comprising a generally circular rim portion 32 can provide advantages over the plurality of wells 26 comprising a generally square-shaped rim portion 38. In some embodiments, during heating, it has been found that assay 1000 can migrate through capillary action upward along edges of sidewalls 40. This can draw assay 1000 from the center of each of the plurality of wells 26, thereby causing variation in the depth of assay 1000. Variations in the depth of assay 1000 can influence the emission output of assay 1000 during analysis. Additionally, during manufacture of microplate 20, in some cases cylindrically shaped mold pins used to form the plurality of wells 26 comprising generally circular rim portion 32 can permit unencumbered flow of molten polymer thereabout. This unencumbered flow of molten polymer results in less deleterious polymer molecule orientation. In some embodiments, generally circular rim portion 32 provides more surface area along microplate 20 for improved sealing with sealing cover 80, as is discussed herein.

In some embodiments, the area of each material retention region can be from about 0.01 to about 0.05 mm². In some embodiments, the width of each material retention region can be from about 200 to about 2,000 microns, or from about 800 to about 3000 microns. In some embodiments, the depth of each material retention region can be about 1100 microns, or about 850 microns. In some embodiments, the surface area of each material retention region can be from about 0.01 to about 0.05 mm², or from about 0.02 to about 0.04 mm². In some embodiments, the aspect ratio (ratio of depth:width) of each material retention region can be from about 1 to about 4, or about 2.

In some embodiments, the volume of the material retention regions can be less than about 50 μl, or less than about 10 μl. In some embodiments, the volume can be from about 0.05 to about 500 nanoliters, from about 0.1 to about 200 nanoliters, from about 20 to about 150 nanoliters, from about 80 to about 120 nanoliters, from about 50 to about 100 nanoliters, from about 1 to about 5 nanoliters, or less than about 2 nanoliters.

### Through-Hole Material Retention Regions

As illustrated in FIGS. 10, 33, and 36, in some embodiments, each of the material retention regions of microplate 20 can comprise a plurality of apertures 48 being sealed at least on one end by sealing cover 80. In some embodiments, each of the plurality of apertures 48 can be sealed on an opposing end with a backing sheet 50, which can have a clear or opaque adhesive. In some embodiments, backing sheet 50 can comprise a heat conducting material such as, for example, a metal foil or a metal coated plastic. In some embodiments, backing sheet 50 can be placed against thermocycler block 102 to aid in thermal conductivity and distribution. In some embodiments, backing sheet 50 can comprise a plurality of reaction spots (as discussed herein), coated on discrete areas of the surface of backing sheet 50, such that in some circumstances the plurality of reaction spots can be aligned with the plurality of apertures 48.

In some embodiments, a layer of mineral oil can be placed at the top of each of the plurality of apertures 48 before, or as an alternative to, placement of sealing cover 80 on microplate 20. In several of such embodiments, the mineral oil can fill a portion of each of the plurality of apertures 48 and provide an optical interface and can control evaporation of assay 1000.

### Grooves

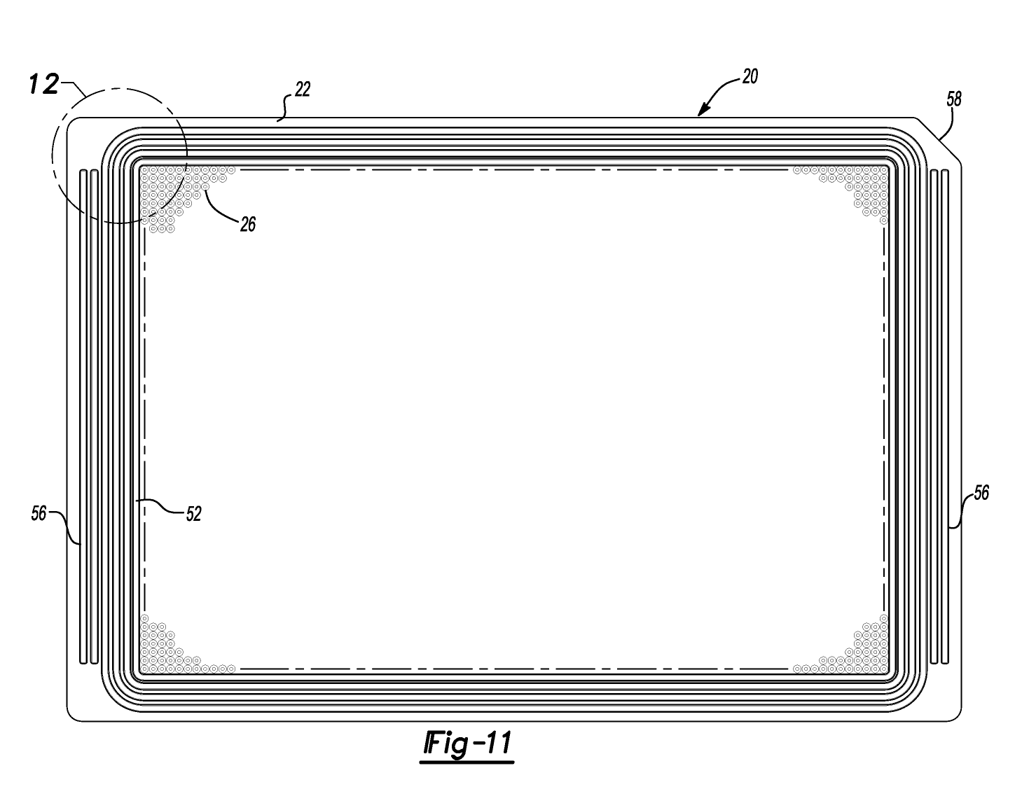

Referring to FIGS. 11-15, in some embodiments, microplate 20 can comprise grooves 52 and grooves 54 disposed about a periphery of the plurality of wells 26. In some embodiments, grooves 52 can have depth and width dimensions generally similar to the depth and width dimensions of the plurality of wells 26 (FIGS. 12 and 13). In some embodiments, grooves 54 can have depth and width dimensions less than the depth and width dimensions of the plurality of wells 26 (FIGS. 14 and 15). In some embodiments, as illustrated in FIG. 12, additional grooves 56 can be disposed at opposing sides of microplate 20. In some embodiments, grooves 52, 54, and 56 can improve thermal uniformity among the plurality of wells 26 in microplate 20. In some embodiments, grooves 52, 54, and 56 can improve the sealing interface formed by sealing cover 80 and microplate 20. Grooves 52, 54, and 56 can also assist in simplifying the injection molding process of microplate 20. In some embodiments, a liquid solution similar to assay 1000 can be disposed in grooves 52, 54, and 56 to, in part, improve thermal uniformity during thermocycling.

### Alignment Features

In some embodiments, as illustrated in FIGS. 2, 3, 11, and 14, microplate 20 comprises an alignment feature 58, such as a corner chamfer, a pin, a slot, a cut corner, an indentation, a graphic, or other unique feature that is capable of interfacing with a corresponding feature formed in a fixture, reagent dispensing equipment, and/or thermocycler. In some embodiments, alignment feature 58 comprises a nub or protrusion 60 as illustrated in FIG. 14. Additionally, in some embodiments, alignment features 58 are placed such that they do not interfere with sealing cover 80 or at least one of the plurality of wells 26. However, locating alignment features 58 near at least one of the plurality of wells 26 can provide improved alignment with dispensing equipment and/or thermocycler block 102.

### Thermally Isolated Portion

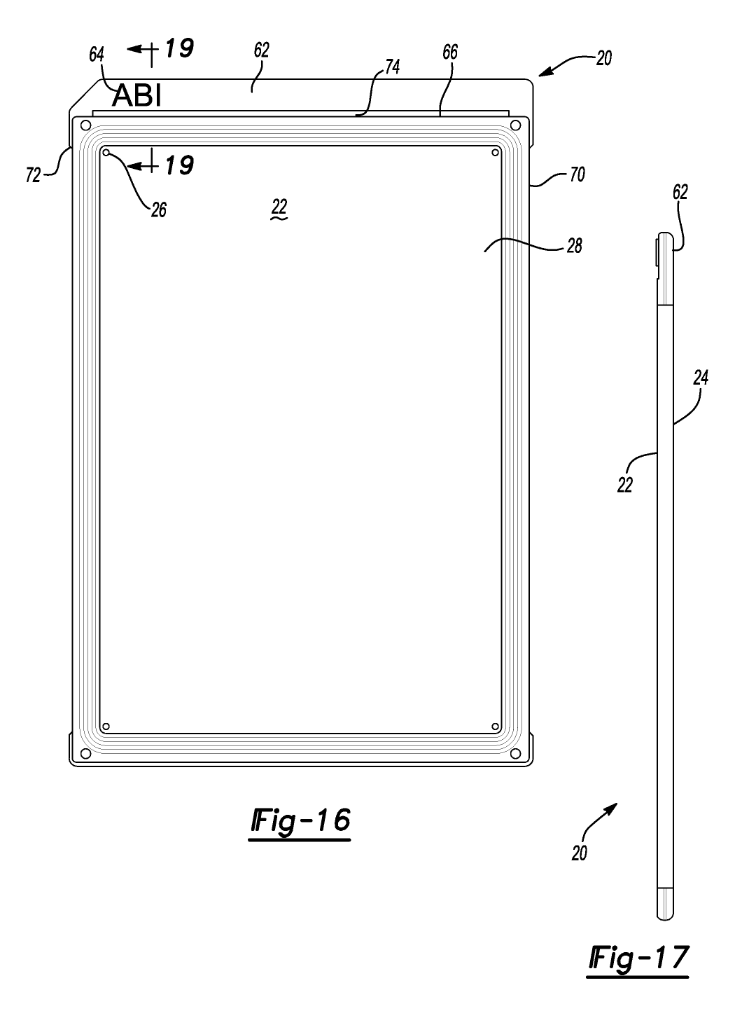

In some embodiments, as illustrated in FIGS. 16-19, microplate 20 comprises a thermally isolated portion 62. Thermally isolated portion 62 can be disposed along at least one edge of main body 28. Thermally isolated portion 62 can be generally free of wells 26 and can be sized to receive a marking indicia 64 (discussed in detail herein) thereon. Thermally isolated portion 62 can further be sized to facilitate the handling of microplate 20 by providing an area that can be easily gripped by a user or mechanical device without disrupting the plurality of wells 26.

Still referring to FIGS. 16-19, in some embodiments, microplate 20 comprises a first groove 66 formed along first surface 22 and a second groove 68 formed along an opposing second surface 24 of microplate 20. First groove 66 and second groove 68 can be aligned with respect to each other to extend generally across microplate 20 from a first side 70 to a second side 72. First groove 66 and second groove 68 can be further aligned upon first surface 22 and second surface 24 to define a reduced cross-section 74 between thermally isolated portion 62 and the plurality of wells 26. This reduced cross-section 74 can provide a thermal isolation barrier to reduce any heat sink effect introduced by thermally isolated portion 62, which might otherwise reduce the temperature cycle of some of the plurality of wells 26.

### Microplate Material

In some embodiments, microplate 20 can comprise, at least in part, a thermally conductive material. In some embodiments, a microplate, in accordance with the present teachings, can be molded, at least in part, of a thermally conductive material to define a cross-plane thermal conductivity of at least about 0.30 W/mK or, in some embodiments, at least about 0.58 W/mK. Such thermally conductive materials can provide a variety of benefits, such as, in some cases, improved heat distribution throughout microplate 20, so as to afford reliable and consistent heating and/or cooling of assay 1000. In some embodiments, this thermally conductive material comprises a plastic formulated for increased thermal conductivity. Such thermally conductive materials can comprise, for example and without limitation, at least one of polypropylene, polystyrene, polyethylene, polyethyleneterephthalate, styrene, acrylonitrile, cyclic polyolefin, syndiotactic polystyrene, polycarbonate, liquid crystal polymer, conductive fillers or plastic materials; and mixtures or combinations thereof. In some embodiments, such thermally conductive materials include those known to those skilled in the art with a melting point greater than about 130° C. For example, microplate 20 can be made of commercially available materials such as RTP199X104849, COOLPOLY E1201, or, in some embodiments, a mixture of about 80% RTP199X104849 and 20% polypropylene.

In some embodiments, microplate 20 can comprise at least one carbon filler, such as carbon, graphite, impervious graphite, and mixtures or combinations thereof. In some cases, graphite has an advantage of being readily and cheaply available in a variety of shapes and sizes. One skilled in the art will recognize that impervious graphite can be non-porous and solvent-resistant. Progressively refined grades of graphite or impervious graphite can provide, in some cases, a more consistent thermal conductivity.

In some embodiments, one or more thermally conductive ceramic fillers can be used, at least in part, to form microplate 20. In some embodiments, the thermally conductive ceramic fillers can comprise boron nitrate, boron nitride, boron carbide, silicon nitride, aluminum nitride, and mixtures or combinations thereof.

In some embodiments, microplate 20 can comprise an inert thermally conductive coating. In some embodiments, such coatings can include metals or metal oxides, such as copper, nickel, steel, silver, platinum, gold, copper, iron, titanium, alumina, magnesium oxide, zinc oxide, titanium oxide, and mixtures thereof.

In some embodiments, microplate 20 comprises a mixture of a thermally conductive material and other materials, such as non-thermally conductive materials or insulators. In some embodiments, the non-thermally conductive material comprises glass, ceramic, silicon, standard plastic, or a plastic compound, such as a resin or polymer, and mixtures thereof to define a cross-plane thermal conductivity of below about 0.30 W/mK. In some embodiments, the thermally conductive material can be mixed with liquid crystal polymers (LCP), such as wholly aromatic polyesters, aromatic-aliphatic polyesters, wholly aromatic poly(ester-amides), aromatic-aliphatic poly(ester-amides), aromatic polyazomethines, aromatic polyester-carbonates, and mixtures thereof. In some embodiments, the composition of microplate 20 can comprise from about 30% to about 60%, or from about 38% to about 48% by weight, of the thermally conductive material.

The thermally conductive material and/or non-thermally conductive material can be in the form of, for example, powder particles, granular powder, whiskers, flakes, fibers, nanotubes, plates, rice, strands, hexagonal or spherical-like shapes, or any combination thereof. In some embodiments, the microplate comprises thermally conductive additives having different shapes to contribute to an overall thermal conductivity that is higher than any one of the individual additives alone.

In some embodiments, the thermally conductive material comprises a powder. In some embodiments, the particle size used herein can be between 0.10 micron and 300 microns. When mixed homogeneously with a resin in some embodiments, powders provide uniform (i.e. isotropic) thermal conductivity in all directions throughout the composition of the microplate.

As discussed above, in some embodiments, the thermally conductive material can be in the form of flakes. In some such embodiments, the flakes can be irregularly shaped particles produced by, for example, rough grinding to a desired mesh size or the size of mesh through which the flakes can pass. In some embodiments, the flake size can be between 1 micron and 200 microns. Homogenous compositions containing flakes can, in some cases, provide uniform thermal conductivity in all directions.

In some embodiments, the thermally conductive material can be in the form of fibers, also known as rods. Fibers can be described, among other ways, by their lengths and diameters. In some embodiments, the length of the fibers can be, for example, between 2 mm and 15 mm. The diameter of the fibers can be, for example, between 1 mm and 5 mm. Formulations that include fibers in the composition can, in some cases, have the benefit of reinforcing the resin for improved material strength.

In some embodiments, microplate 20 can comprise a material comprising additives to promote other desirable properties. In some embodiments, these additives can comprise flame-retardants, antioxidants, plasticizers, dispersing aids, marking additives, and mold-releasing agents. In some embodiments, such additives are biologically and/or chemically inert.

In some embodiments, microplate 20 comprises, at least in part, an electrically conductive material, which can improve reagent dispensing alignment. In this regard, electrically conductive material can reduce static build-up on microplate 20 so that the reagent droplets will not go astray during dispensing. In some embodiments, a voltage can be applied to microplate 20 to pull the reagent droplets into a predetermined position, particularly with a co-molded part where the bottom section can be electrically conductive and the sides of the plurality of wells 26 may not be electrically conductive. In some embodiments, a voltage field applied to the electrically conductive material under the well or wells of interest can pull assay 1000 into the appropriate wells.

In some embodiments, microplate 20 can be made, at least in part, of non-electrically conductive materials. In some embodiments, non-electrically conductive materials can at least in part comprise one or more of crystalline silica (3.0 W/mK), aluminum oxide (42 W/mK), diamond (2000 W/mK), aluminum nitride (150-220 W/mK), crystalline boron nitride (1300 W/mK), and silicon carbide (85 W/mK).

### Microplate Surface Treatments

In some embodiments, the surface of the microplate 20 comprises an enhanced surface which can comprise a physical or chemical modality on or in the surface of the microplate so as to enhance support of, or filling of, assay 1000 in a material retention region (e.g., a well or a reaction spot). Such modifications can include chemical treatment of the surface, or coating the surface. In some embodiments, such chemical treatment can comprise chemical treatment or modification of the surface of the microplate so as to form relatively hydrophilic and hydrophobic areas. In some embodiments, a surface tension array can be formed comprising a pattern of hydrophilic sites forming material retention regions on an otherwise hydrophobic surface, such that the hydrophilic sites can be spatially segregated by hydrophobic areas. Reagents delivered to the surface tension array can be retained by surface tension difference between the hydrophilic sites and the hydrophobic areas.

In some embodiments, hydrophobic areas can be formed on the surface of microplate 20 by coating microplate 20 with a photoresist substance and using a photomask to define a pattern of material retention regions on microplate 20. After exposure of the photomasked pattern, at least a portion of the surface of microplate 20 can be reacted with a suitable reagent to form a stable hydrophobic surface. Such reagents can comprise, for example, one or more members of alkyl groups, such as, for example, fluoroalkylsilane or long chain alkylsilane (e.g octadecylsilane). The remaining photoresist substance can then be removed and the solid support reacted with a suitable reagent, such as aminoalkyl silane or hydroxyalkyl silane, to form hydrophilic sites. In some embodiments, microplate 20 can be first reacted with a suitable derivatizing reagent to form a hydrophobic surface. Such reagents can comprise, for example, vapor or liquid treatment of fluoroalkylsiloxane or alkylsilane. The hydrophobic surface can then be coated with a photoresist substance, photopatterned, and developed.

In some embodiments, the exposed hydrophobic surface can be reacted with suitable derivatizing reagents to form hydrophilic sites. For example, in some embodiments, the exposed hydrophobic surface can be removed by wet or dry etch such as, for example, oxygen plasma and then derivatized by aminoalkylsilane or hydroxylalkylsilane treatment. The photoresist coat can then be removed to expose the underlying hydrophobic areas.

The exposed surface can be reacted with suitable derivatizing reagents to form hydrophobic areas. In some embodiments, the hydrophobic areas can be formed by fluoroalkylsiloxane or alkylsilane treatment. The photoresist coat can be removed to expose the underlying hydrophilic sites. In some embodiments, fluoroalkylsilane or alkylsilane can be employed to form a hydrophobic surface. In some embodiments, aminoalkyl silane or hydroxyalkyl silane can be used to form hydrophilic sites. In some embodiments, derivatizing reagents can comprise hydroxyalkyl siloxanes, such as allyl trichlorochlorosilane, and 7-oct-I-enyl trichlorochlorosilane; diol (bis-hydroxyalkyl) siloxanes; glycidyl trimethoxysilanes; aminoalkyl siloxanes, such as 3-aminopropyl trimethoxysilane; Dimeric secondary aminoalkyl siloxanes, such as bis (3-trimethoxysilylpropyl) amine; and combinations thereof.

In some embodiments, the surface of microplate 20 can be first reacted with a suitable derivatizing reagent to form hydrophilic sites. Suitable reagents can comprise, for example, vapor or liquid treatment of aminoalkylsilane or hydroxylalkylsilane. The derivatized surface can then be coated with a photoresist substance, photopatterned, and developed. In some embodiments, hydrophilic sites can be formed on the surface of microplate 20 by forming the surface, or chemically treating it, with compounds comprising free amino, hydroxyl, carboxyl, thiol, amido, halo, or sulfate groups. In some embodiments, the free amino, hydroxyl, carboxyl, thiol, amido, halo, or sulfate group of the hydrophilic sites can be covalently coupled with a linker moiety (e. g., polylysine, hexethylene glycol, and polyethylene glycol).

In some embodiments, hydrophilic sites and hydrophobic areas can be made without the use of photoresist. In some embodiments, a substrate can be first reacted with a reagent to form hydrophilic sites. At least some the hydrophilic sites can be protected with a suitable protecting agent. The remaining, unprotected, hydrophilic sites can be reacted with a reagent to form hydrophobic areas. The protected hydrophilic sites can then be unprotected. In some embodiments, a glass surface can be reacted with a reagent to generate free hydroxyl or amino sites. These hydrophilic sites can be reacted with a protected nucleoside coupling reagent or a linker to protect selected hydroxyl or amino sites. In some embodiments, nucleotide coupling reagents can comprise, for example, a DMT-protected nucleoside phosphoramidite, and DMT-protected H-phosphonate. The unprotected hydroxyl or amino sites can be reacted with a reagent, for example, perfluoroalkanoyl halide, to form hydrophobic areas. The protected hydrophilic sites can then be unprotected.

In some embodiments, the chemical modality can comprise chemical treatment or modification of the surface of microplate 20 so as to anchor one or more components of assay 1000 to the surface. In some embodiments, one or more components of assay 1000 can be anchored to the surface so as to form a patterned immobilization reagent array of material retention regions. In some embodiments, the immobilization reagent array can comprise a hydrogel affixed to microplate 20. In some embodiments, hydrogels can comprise cellulose gels, such as agarose and derivatized agarose; xanthan gels; synthetic hydrophilic polymers, such as crosslinked polyethylene glycol, polydimethyl acrylamide, polyacrylamide, polyacrylic acid (e.g., cross-linked with dysfunctional monomers or radiation cross-linking), and micellar networks; and mixtures thereof. In some embodiments, derivatized agarose can comprise agarose which has been chemically modified to alter its chemical or physical properties. In some embodiments, derivatized agarose can comprise low melting agarose, monoclonal anti-biotin agarose, streptavidin derivatized agarose, or any combination thereof.

In some embodiments, an anchor can be an attachment of a reagent to the surface, directly or indirectly, so that one or more reagents is available for reaction during a chemical or amplification method, but is not removed or otherwise displaced from the surface prior to reaction during routine handling of the substrate and sample preparation prior to use. In some embodiments, assay 1000 can be anchored by covalent or non-covalent bonding directly to the surface of the substrate. In some embodiments, assay 1000 can be bonded, anchored, or tethered to a second moiety (immobilization moiety) which, in turn, can be anchored to the surface of microplate 20. In some embodiments, assay 1000 can be anchored to the surface through a chemically releasable or cleavable site, for example by bonding to an immobilization moiety with a releasable site. Assay 1000 can be released from microplate 20 upon reacting with cleaving reagents prior to, during, or after manufacturing of microplate 20. Such release methods can include a variety of enzymatic, or non-enzymatic means, such as chemical, thermal, or photolytic treatment.

In some embodiments, assay 1000 can comprise a primer, which is releasable from the surface of microplate 20. In some embodiments, a primer can be initially hybridized to a polynucleotide immobilization moiety, and subsequently released by strand separation from the array-immobilized polynucleotides during manufacturing of microplate 20. In some embodiments, a primer can be covalently immobilized on microplate 20 via a cleavable site and released before, during, or after manufacturing of microplate 20. For example, an immobilization moiety can contain a cleavable site and a primer. The primer can be released via selective cleavage of the cleavable sites before, during, or after assembly. In some embodiments, the immobilization moiety can be a polynucleotide which contains one or more cleavable sites and one or more primer polynucleotides. A cleavable site can be introduced in an immobilized moiety during in situ synthesis. Alternatively, the immobilized moieties containing releasable sites can be prepared before they are covalently or noncovalently immobilized on the solid support. In some embodiments, chemical moieties for immobilization attachment to solid support can comprise carbamate, ester, amide, thiolester, (N)-functionalized thiourea, functionalized maleimide, amino, disulfide, amide, hydrazone, streptavidin, avidin/biotin, and gold-sulfide groups.

In some embodiments, microplate 20 can be coated with one or more thin conformal isotropic coatings operable to improve the surface characteristics of the microplate, the material retention regions, or both, for conducting a chemical or amplification reaction. In some embodiments, such treatments improve wettability of the surface, low moisture transmissivity of the surface, and high service temperature characteristics of the substrate.

### Microplate Spotting, Filling, and Sealing

In some embodiments, one or more devices can be used to facilitate the placement of one or more components of assay 1000 within at least some of the plurality of wells 26 of microplate 20.

In some embodiments, microplate 20 can additionally comprise a filling feature, which is operable to facilitate filling of reagents and/or samples into the material retention regions of microplate. In some embodiments, filling devices can include, for example, physical and chemical modalities that direct, channel, route, or otherwise effect flow of reagents or samples on the surface of microplate 20, on the surface of sealing cover 80, or combinations thereof. In some embodiments, the filling device effects flow of reagents into material retention regions. In some embodiments, microplate 20 can comprise raised or depressed regions (e. g., barriers and trenches) to aid in the distribution and flow of liquids on the surface of the microplate. In some embodiments, the filling system comprises capillary channels. The dimensions of these features are flexible, depending on factors, such as avoidance of air bubbles during use, handling convenience, and manufacturing feasibility.

In some embodiments, microplate 20 can additionally comprise a gasket between sealing cover 80 and microplate 20, creating a space between sealing cover 80 and microplate 20. In some embodiments, the gasket can comprise a material which is operable to form a seal between sealing cover 80 and microplate 20. In some embodiments, the gasket comprises one or more ports which are operable to admit a fluid or gas, such as, for example, one or more components of assay 1000 into the space formed between sealing cover 80 and microplate 20.

### Microplate Spotting

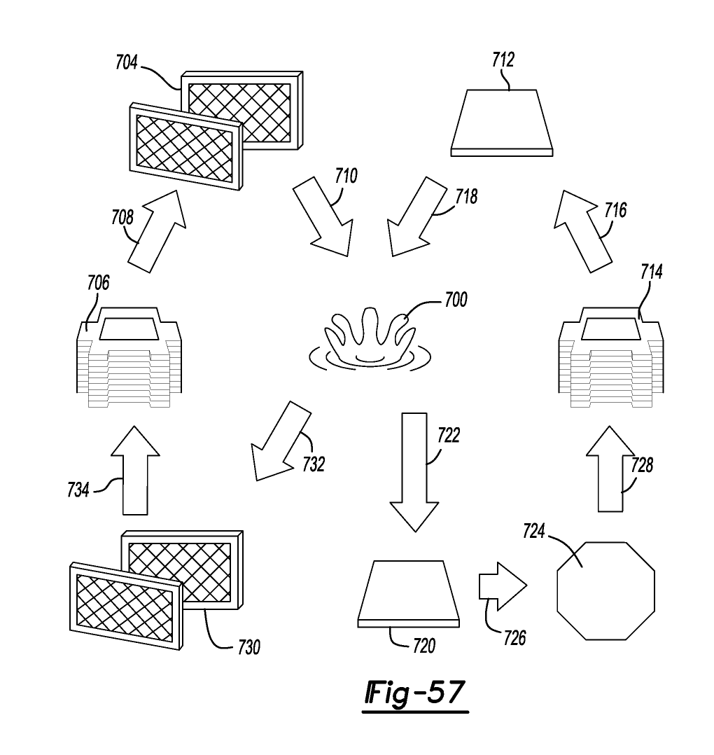

In some embodiments, as illustrated in FIG. 57, microplate 20 can be preloaded with at least some component materials of assay 1000, such as reagents. In some embodiments, as described further herein, such reagents can comprise at least one primer and at least one detection probe. In some embodiments, such reagents can comprise elements facilitating analysis of a whole genome or a portion of a genome. Still further, in some embodiments, such reagents can comprise buffers and/or additives useful for coating, stability, enhanced rehydration, preservation, and/or enhanced dispensing of reagents.

In some embodiments, such reagents can be delivered (e.g. spotted) into at least one of the plurality of wells 26 of microplate 20 in very small, e.g. nanoliter, increments using a spotting device 700 (FIG. 8). In some embodiments, spotting device 700 employs one or more piezoelectric pumps, acoustic dispersion, liquid printers, micropiezo dispensers, or the like to deliver such reagents to each of the plurality of wells. In some embodiments, spotting device 700 employs an apparatus and method like or similar to that described in commonly assigned U.S. Pat. Nos. 6,296,702, 6,440,217, 6,579,367, and 6,849,127, issued to Vann et al.

According to some embodiments, in operation, as schematically illustrated in FIG. 57, reagents, e.g. in an aqueous form or bead form, can be stored on one or more storage plates 704 in a high-humidity storage unit 706. In some embodiments, high-humidity storage unit 706 can comprise a relative humidity in the range of about 70-100%. However, in some embodiments, high-humidity storage unit 706 can comprise a relative humidity in the range of about 70-85%. The bead form can be like or similar to that described in commonly assigned U.S. Pat. No. 6,432,719 to Vann et al. Some of the plurality of storage plates 704 can be moved out of high-humidity storage unit 706, as indicated by 708, and can be placed onto spotting device 700, as indicated by 710. A separate unspotted microplate 712 can then be moved out of a low-humidity storage unit 714, as indicated by 716. In some embodiments, low-humidity storage unit 714 can comprise a relative humidity in the range of about 0-30%. Unspotted microplate 712 can then be placed on spotting device 700, as indicated by 718. Reagents from storage plate 704 can then be spotted onto at least some of the plurality of wells 26 on unspotted microplate 712. Once at least some of the plurality of wells 26 are spotted, the spotted microplate 720 can then be moved from spotting device 700, as indicated by 722. Spotted microplate 720 can then be moved to an optional quality-control station 724, as indicated by 726. After quality-control station 724, spotted microplate 720 can then be moved back to low-humidity storage unit 714, as indicated by 728. This procedure of spotting microplates 20 can continue until a desired number (e.g. all) of microplates in storage unit 714 have been spotted with reagents from storage plate 704. It should be noted that unspotted microplate 712 and spotted microplate 720 are each similar to microplate 20, however different numerals are used for simplicity in the above description.

In some embodiments, the spots of reagents on spotted microplate 720 can be partially or fully dried down, as desired, in the low-humidity of storage unit 714. In some embodiments, storage unit 714 can also be heated to facilitate this drying. Once the microplates from storage unit 714 have been spotted with reagents from storage plate 704, storage plate 704 can be removed and designated as a used storage plate 730. Used storage plate 730 can be removed from spotting device 700 as indicated by 732. Used storage plate 730 can be returned to high-humidity storage unit 706 as indicated by 734. The process can continue as the next storage plate 704 is moved out of high-humidity storage unit 706 and into spotting device 700. In some embodiments, this next storage plate 704 can contain a different set of reagents. The aforementioned process can then be repeated, as desired. This process can continue until all of the plurality of wells 26 on spotted microplate 720 have been spotted or, in some cases, a portion of the plurality of wells 26 have been spotted, while leaving the remaining wells 26 empty.

It should be appreciated that this preloading process can vary as desired to accommodate user needs. For instance, in some embodiments, the reagents spotted in each of the plurality of wells 26 can be encapsulated with a material. Such encapsulation can prevent or reduce moisture at room temperature from interacting with the reagents. In some embodiments, each of the plurality of wells 26 can be spotted several times with reagents, such as for multiplex PCR. In some embodiments, these multiple spotted reagents can form layers. In some embodiments of this preloading process, primer sets and detection probes for a whole genome can be spotted from storage plates 704 onto spotted microplate 720. In other embodiments, a portion of a genome, or subsets of selected genes, can be spotted from source plates 704 onto spotted microplate 720.

In some embodiments, spotted microplate 720 can be sealed with a protective cover, stored, and/or shipped to another location. In some embodiments, the protective cover is releasable from spotted microplate 720 in one piece without leaving adhesive residue on spotted microplate 720. In some embodiments, the protective cover is visibly different (e.g., a different color) from sealing cover 80 to aid in visual identification and for ease of handling.

In some embodiments, the protective cover can be made of a material chosen to reduce static charge generation upon release from spotted microplate 720. When it is time for spotted microplate 720 to be used, the package seal can be broken and the protective cover can be removed from spotted microplate 720. In some embodiments, the protective cover can be a pierceable film, a slitted film, or a duckbilled closure to, at least in part, reduce contamination and/or evaporation. An analyte (such a biological sample comprising DNA) can then be added to spotted microplate 720, along with other materials such as PCR master mix, to form assay 1000 in at least some of the plurality of wells 26. Spotted microplate 720 can then be sealed with sealing cover 80 as described above. High-density sequence detection system 10 can then be actuated to collect and analyze data.

In some embodiments, the filling apparatus comprises a device for depositing (e.g., spotting or spraying) of assay 1000 to specific wells, wherein one or more of the plurality of wells 26 of microplate 20 contains a different assay material than other wells 26 of microplate 20. In some embodiments, the device can include piezoelectric pumps, acoustic dispersion, liquid printers, or the like. According to some embodiments, a pin spotter can be employed, such as described in PCT Publication No. WO 2004/018104. In some embodiments, a fiber and/or fiber-array spotter can be employed, such as described in U.S. Pat. No. 6,849,127.

In some embodiments, the filling apparatus comprises a device for depositing assay 1000 to a plurality of wells, wherein two or more wells contain the same assay material. In some embodiments, microplate 20 comprises two more groups of wells 26. Each of the groups of wells 26 can comprise a different assay material than at least one other group of wells 26 on microplate 20.

### Microplate Filling

In some embodiments, a filling apparatus 400 can be used to fill at least some of the plurality of wells 26 of microplate 20 with one or more components of assay 1000. It should be understood that filling apparatus 400 can comprise any one of a number of configurations.

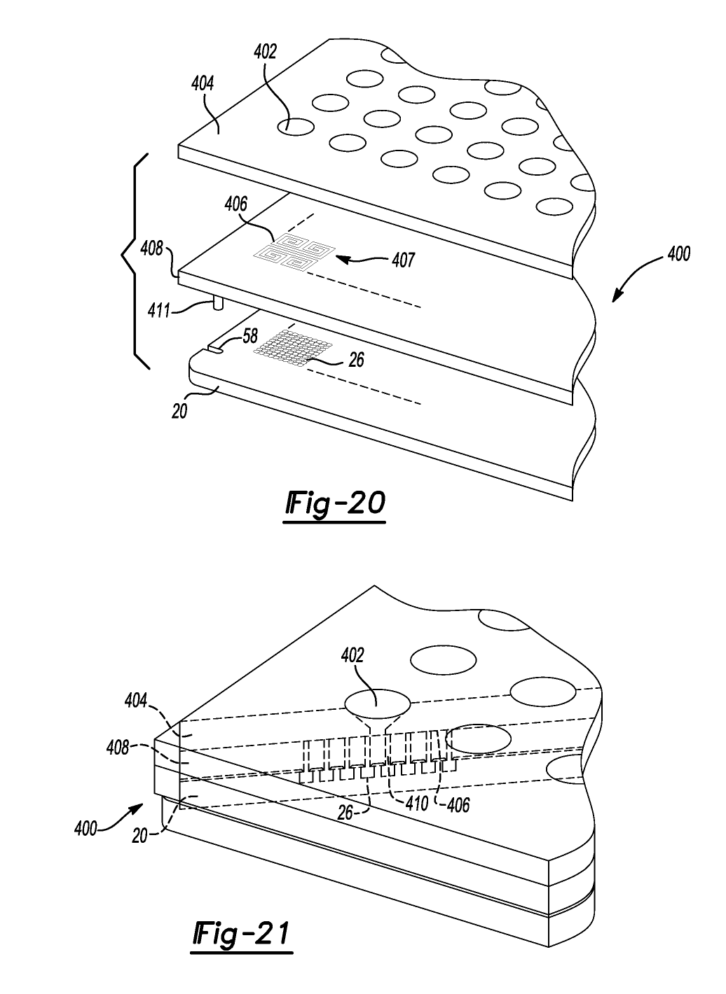

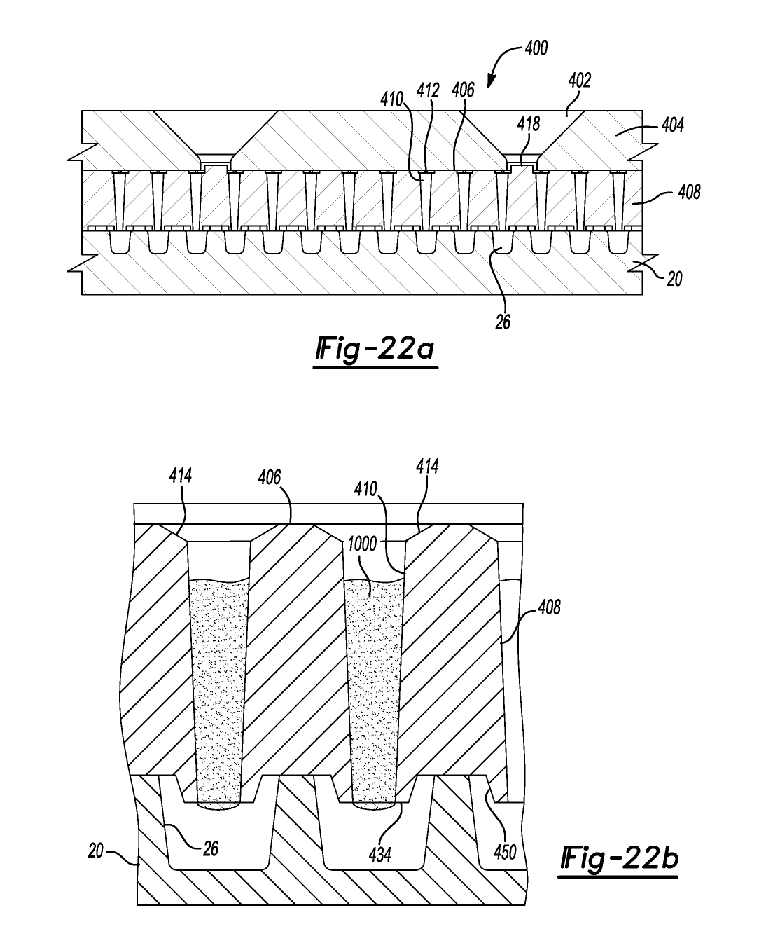

In some embodiments, referring to FIGS. 20-22(b), filling apparatus 400 comprises one or more assay input ports 402, such as about 96 input ports, disposed in an input layer 404. In some embodiments, assay input ports 402 of input layer 404 can be in fluid communication with a plurality of microfluidic channels 406 disposed in input layer 404, an output layer 408, or any other layer of filing apparatus 400. In some embodiments, the plurality of microfluidic channels 406 can be formed in an underside of input layer 404 and a seal member can be placed over the underside of input layer 404. In some embodiments, the seal member can comprise a perforation (e.g. hole) positioned over a desired location in microplate 20 to permit a discrete fluid communication passage to extend therethrough. In some embodiments, the plurality of microfluidic channels 406 can be arranged as a grouping 407 (FIG. 20). In some embodiments, assay input ports 402 can be positioned at a predetermined pitch (e.g. 9 mm) such that each assay input port 402 can be aligned with a center of each grouping 407. In some embodiments, the plurality of microfluidic channels 406 can be in fluid communication with a plurality of staging capillaries 410 formed in output layer 408 (FIGS. 21-22(b)).

During filling, assay 1000 can be put into at least one assay input port 402 and can be fluidly channeled toward at least one of the plurality of microfluidic channels 406, first passing a surface tension relief post 418 in some embodiments. In some embodiments, surface tension relief post 418 can serve, at least in part, to evenly spread assay 1000 throughout the plurality of microfluidic channels 406 and/or engage a meniscus of assay 1000 to encourage fluid flow. Assay 1000 can be fluidly channeled through the plurality of microfluidic channels 406 and can collect in the plurality of staging capillaries 410 (FIG. 22(b)). Assay 1000 can then be held in the plurality of staging capillaries 410 by capillary or surface tension forces.

In some embodiments, as illustrated in FIGS. 21 and 22(a)-(b), microplate 20 can be attached to filling apparatus 400 so that each of the plurality of staging capillaries 410 is generally aligned with each of the plurality of wells 26. In some embodiments, filling apparatus 400 comprises alignment features 411 (FIG. 20) operably sized to engage corresponding alignment feature 58 on microplate 20 to, at least in part, facilitate proper alignment of each of the plurality of staging capillaries 410 with a corresponding (respective) one of the plurality of wells 26. In some embodiments, the combined unit of filling apparatus 400 and microplate 20 can then be placed in a centrifuge. The centrifugal force of the centrifuge can, at least in part, urge assay 1000 from the plurality of staging capillaries 410 into each of the plurality of wells 26 of microplate 20. Filling apparatus 400 can then be removed from microplate 20. In some embodiments, microplate 20 can then receive additional reagents and/or be sealed with sealing cover 80, or other sealing feature such as a layer of mineral oil, and then placed into high-density sequence detection system 10.

In some embodiments, capillary or surface tension forces encourage flow of assay 1000 through staging capillaries 410. In this regard, staging capillaries 410 can be of capillary size, for example, staging capillaries 410 can be formed with an exit diameter less than about 500 micron, and in some embodiments less than about 250 microns. In some embodiments, staging capillaries 410 can be formed, for example, with a draft angle of about 1-5° and can define any thickness sufficient to achieve a predetermined volume. To further encourage the desired capillary action in staging capillaries 410, staging capillaries 410 can be provided with an interior surface that is hydrophilic, i.e., wettable. For example, the interior surface of staging capillaries 410 can be formed of a hydrophilic material and/or treated to exhibit hydrophilic characteristics. In some embodiments, the interior surface comprises native, bound, or covalently attached charged groups. For example, one suitable surface, according to some embodiments, is a glass surface having an absorbed layer of a polycationic polymer, such as poly-I-lysine.

### Microplate Sealing Cover

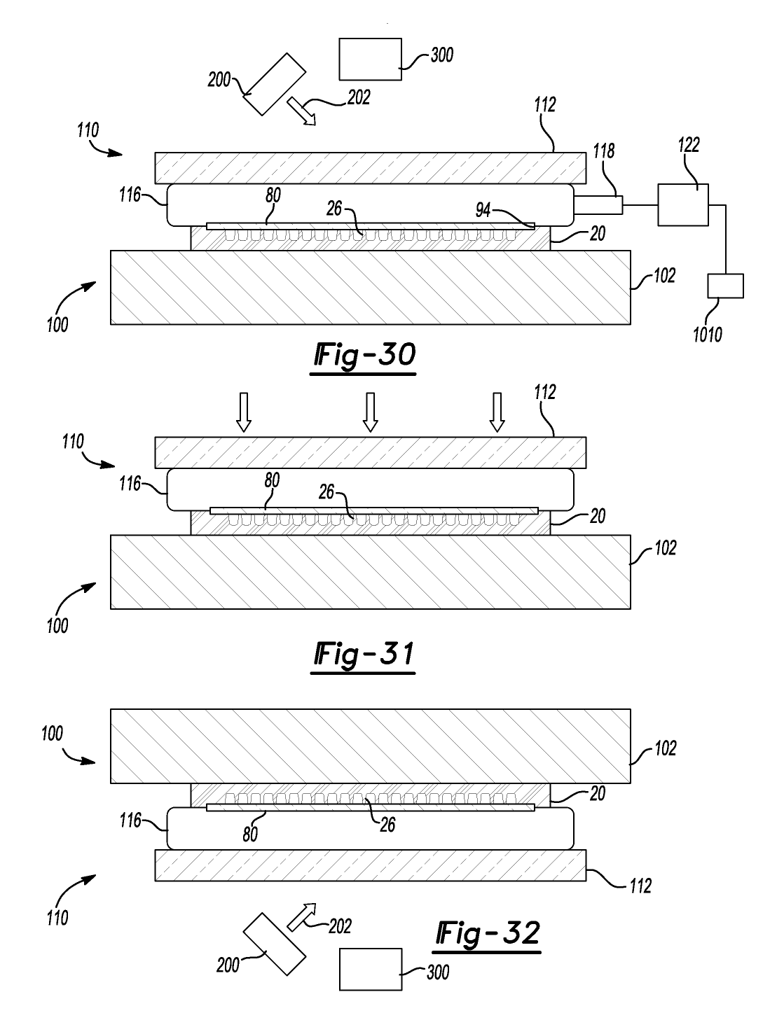

In some embodiments, such as illustrated in FIGS. 26 and 27, sealing cover 80 can be generally disposed across microplate 20 to seal assay 1000 within each of the plurality of wells 26 of microplate 20 along a sealing interface 92 (see FIGS. 4, 5, 26, and 27). That is, sealing cover 80 can seal (isoloate) each of the plurality of wells 26 and its contents (i.e. assay 1000) from adjacent wells 26, thus maintaining sample integrity between each of the plurality of wells 26 and reducing the likelihood of cross contamination between wells. In some embodiments, sealing cover 80 can be positioned within an optional depression 94 (FIG. 30) formed in main body 28 of microplate 20 to promote proper positioning of sealing cover 80 relative to the plurality of wells 26.

In some embodiments, sealing cover 80 can be made of any material conducive to the particular processing to be done. In some embodiments, sealing cover 80 can comprise a durable, generally optically transparent material, such as an optically clear film exhibiting abrasion resistance and low fluorescence when exposed to an excitation light. In some embodiments, sealing cover 80 can comprise glass, silicon, quartz, nylon, polystyrene, polyethylene, polycarbonate, copolymer cyclic olefin, polycyclic olefin, cellulose acetate, polypropylene, polytetrafluoroethylene, metal, and combinations thereof.

In some embodiments, sealing cover 80 comprises an optical element, such as a lens, lenslet, and/or a holographic feature. In some embodiments, sealing cover 80 comprises features or textures operable to interact with (e.g., by interlocking engagement) circular rim portion 32 or square-shaped rim portion 38 of the plurality of wells 26. In some embodiments, sealing cover 80 can provide resistance to distortion, cracking, and/or stretching during installation. In some embodiments, sealing cover 80 can comprise water impermeable-moisture vapor transmission values below 0.5 (cc-mm)/(m2-24 hr-atm). In some embodiments, sealing cover 80 can maintain its physical properties in a temperature range of 4° C. to 99° C. and can be generally free of inclusions (e.g. light blocking specks) greater than 50 μm, scratches, and/or striations. In some embodiments, sealing cover 80 can comprise a liquid such as, for example, oil (e.g., mineral oil).

In some embodiments, such sealing material can comprise one or more compliant coatings and/or one or more adhesives, such as pressure sensitive adhesive (PSA) or hot melt adhesive. In some embodiments, a pressure sensitive adhesive can be readily applied at low temperatures. In some embodiments, the pressure sensitive adhesive can be softened to facilitate the spreading thereof during installation of sealing cover 80. In some embodiments, such sealing maintains sample integrity between each of plurality of wells 26 and prevents wells cross-contamination of contents between wells 26. In some embodiments, adhesive 88 exhibits low fluorescence.

In some embodiments, the sealing material can provide sufficient adhesion between sealing cover 80 and microplate 20 to withstand about 2.0 lbf per inch or at least about 0.9 lbf per inch at 95° C. In some embodiments, the sealing material can provide sufficient adhesion at room temperature to contain assay 1000 within each of the plurality of wells 26. This adhesion can inhibit sample vapor from escaping each of the plurality of wells 26 by either direct evaporation or permeation of water and/or assay 1000 through sealing cover 80. In some embodiments, the sealing material maintains adhesion between sealing cover 80 and microplate 20 in cold storage at 2° C. to 8° C. range (non-freezing conditions) for 48 hours.

In some embodiments, in order to improve sealing of the plurality of wells 26 of microplate 20, various treatments to microplate 20 can be used to enhance the coupling of sealing cover 80 to microplate 20. In some embodiments, microplate 20 can be made of a hydrophobic material or can be treated with a hydrophobic coating, such as, but not limited to, a fluorocarbon, PTFE, or the like. The hydrophobic material or coating can reduce the number of water molecules that compete with the sealing material on sealing cover 80. As discussed above, grooves 52, 54 can be used to provide seal adhesion support on the outer edges of sealing cover 80. In these embodiments, for example, a pressure chamber gasket can be sealed against grooves 52, 54 for improved sealing.

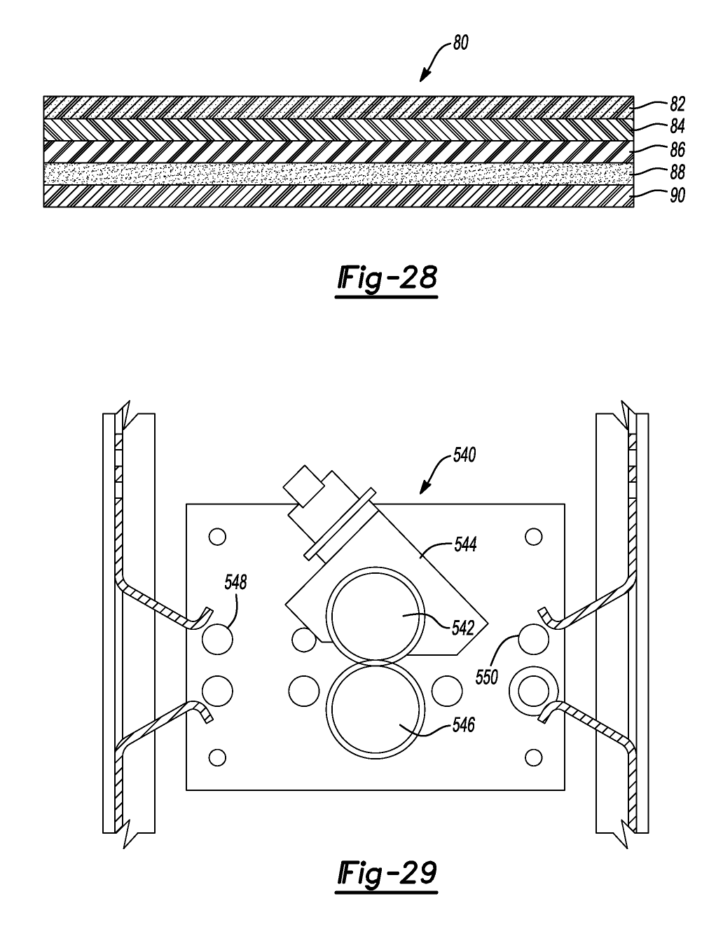

Turning now to FIG. 28, in some embodiments, sealing cover 80 can comprise multiple layers, such as a friction reduction film 82, a base stock 84, a compliant layer 86, a pressure sensitive adhesive 88, and/or a release liner 90. In some embodiments, friction reduction film 82 can be Teflon or a similar friction reduction material that can be peeled off and removed after sealing cover 80 is applied to microplate 20 and before microplate 20 is placed in high-density sequence detection system 10. In some embodiments, base stock 84 can be a scuff resistant and water impermeable layer with low to no fluorescence. While in some embodiments, compliant layer 86 can be a soft silicone elastomer or other material known in the art that is deformable to allow pressure sensitive adhesive 88 to conform to irregular surfaces of microplate 20, increase bond area, and resist delamination of sealing cover 80. In some embodiments, pressure sensitive adhesive 88 and compliant layer 86 can be a single layer, if the pressure sensitive adhesive exhibit sufficient compliancy. Release liner 90 is removed prior to coupling pressure sensitive adhesive 88 to microplate 20.

In some embodiments, sealing cover 80 can comprise a plurality of reaction spots, where the reaction spots are aligned with material retention regions or plurality of wells 26 in microplate 20. In some embodiments, the reaction spots can comprise one or more components of assay 1000, which in some circumstance can alleviate the need for deposition of such one or more components of assay 1000 on the material retention regions or into the plurality of wells 26.

### Compatibility of Cover and Assay

In some embodiments, adhesive 88 can selected so as to be compatible with assay 1000. For example, in some embodiments adhesive 88 is free of nucleases, DNA, RNA and other assay components, as discussed below. In some embodiments, sealing cover 80 comprises one or more materials that are selected so as to be compatible with detection probes in assay 1000. In some embodiments, adhesive layer 88 is selected for compatibility with detection probes.

Methods of matching a detection probe with a compatible sealing cover 80 include, in some embodiments, varying compositions of sealing cover 80 by different weight percents of components such as polymers, crosslinkers, adhesives, resins and the like. These sealing covers 80 can then be tested as a function of their corresponding fluorescent intensity level for different dyes. In such embodiments, comparison can be analyzed at room temperature as well as at elevated temperatures typically employed with PCR. Comparisons can be analyzed over a period of time and in some embodiments, the time period can be, for example, up to 24 hours. Data can be collected for each of the varying compositions of sealing cover 80 and plotted such that fluorescence intensity of the dye is on the X-axis and time is on the Y-axis. Some embodiments of the present teachings include a method of testing compatibility of the detection probe comprising an oligonucleotide and a fluorophore to a composition of a sealing cover. In such embodiments, the method includes depositing a quantity of the fluorophore into a plurality of containers, providing a plurality of sealing covers that have different compositions and sealing the containers with the sealing covers. Methods also include exciting the fluorophore in each of the containers and then measuring an emission intensity from the fluorophore in each of the containers. In such embodiments, the method can also include an evaluation of the emission intensity from the fluorophore of each of the containers and then a determination of which sealing cover composition is compatible with the fluorophore. In some embodiments, the method includes holding a temperature of the containers constant. The method can include measuring the emission intensity from the fluorophore in each container over a period of time, for example, as long as about 24 hours. In some embodiments, the method includes heating the containers to a temperature above about 20° C., optionally to a temperature from about 55° C. to about 100° C. In some embodiments, the method includes cycling the temperature of the plurality of containers. The temperature of the containers can be cycled according to a typical PCR temperature profile. Table 4 shows exemplary data that can be generated for such a comparison. In this example, a dye is evaluated by comparing it at non-heated and heated temperatures to a cyclic olefin copolymer (COC) and glue material with varying percentages of a crosslinker.

TABLE 4
Percentage of Flourescence Signal Loss

| | Percentage of Fluorescence Signal Loss Post | |
|---|---|---|
| | Incubaton with Dye (20 hrs; 59° C.) | |
| | Fresh Material | Material Heated |
| Sealing Cover Composition | (Room Temperature) | (24 hrs; 70° C.) |
| Control (No COC, glue, | 0% Loss | 0% Loss |
| or crosslinker) | | |
| COC/Glue/0% crosslinker | 0% Loss | 0% Loss |
| COC/Glue/0.5% crosslinker | 87% Loss | 76% Loss |
| COC/Glue/1% crosslinker | 86% Loss | 12.5% Loss |
| COC/Glue/3% crosslinker | 55% Loss | 0% Loss |
| COC/Glue/5% crosslinker | 97% Loss | 95% Loss |

In some embodiments, kits are provided, comprising, for example, a sealing cover 80 and one or more compatible detection probes that are compatible (e.g., emission intensity does not degrade when in contact) with sealing cover 80. In some embodiments, a kit can comprise one or more detection probes that are compatible (e.g., do not degrade over time when in contact) with adhesive 88 of sealing cover 80. Kits may comprise a group of detection probes that are compatible with sealing cover 80 comprising adhesive 88 and microplate 20. In some embodiments, the present teachings include methods for matching a group of detection probes that are compatible with sealing cover 80 and spotting into at least some of plurality of wells 26 of microplate 20.

### Microplate Sealing Cover Roll

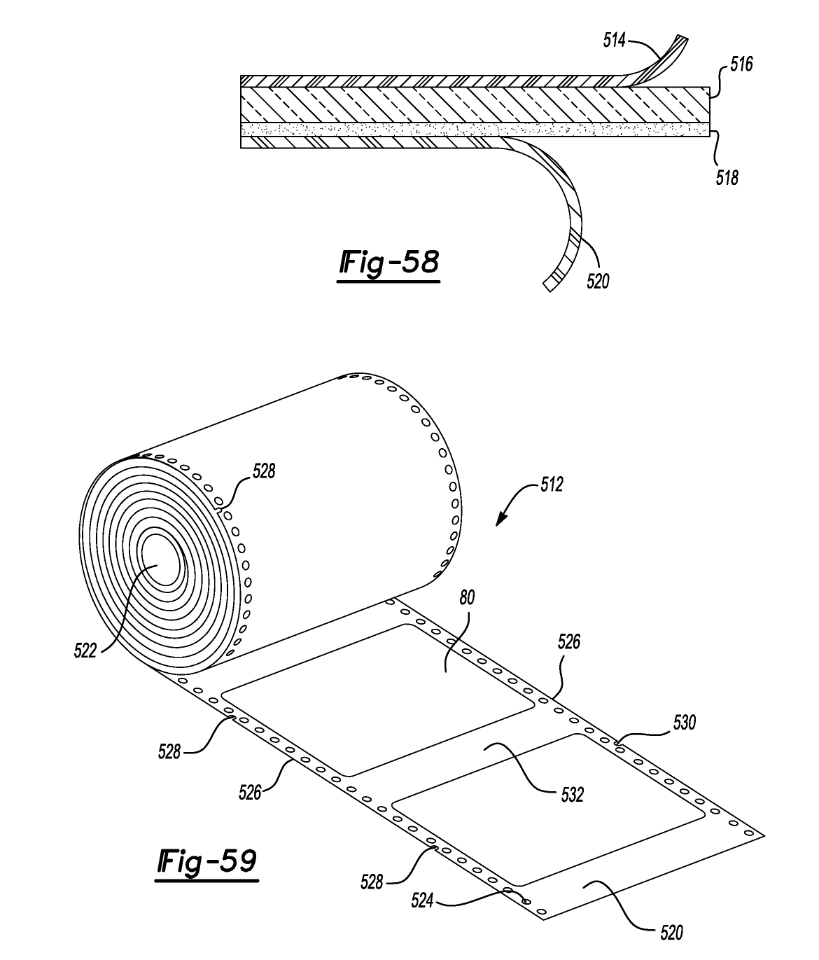

As can be seen in FIGS. 58 and 59, in some of the embodiments, sealing cover 80 can be configured as a roll 512. The use of sealing cover roll 512 can provide, in some embodiments, and circumstances, improved ease in storage and application of sealing cover 80 on microplate 20 when used in conjunction with a manual or automated sealing cover application device, as discussed herein. In some embodiments, sealing cover roll 512 can be manufactured using a laminate comprising a protective liner 514, a base stock 516, an adhesive 518, and/or a carrier liner 520. During manufacturing, protective liner 514 can be removed and discarded. Base stock 516 and adhesive 518 can then be kiss-cut, such that base stock 516 and adhesive 518 are cut to a desired shape of sealing cover 80, yet carrier liner 520 is not cut. Excess portions of base stock 516 and adhesive 518 can then be removed and discarded. In some embodiments, base stock 516 can be a scuff resistant and water impermeable layer with low to no fluorescence.

In some embodiments, carrier liner 520 can then be punched or otherwise cut to a desired shape and finally the combination of carrier liner 520, base stock 516, and adhesive 518 can be rolled about a roll core 522 (see FIG. 59). Roll core 522 can be sized so as not to exceed the elastic limitations of base stock 516, adhesive 518, and/or carrier liner 520. In some embodiments, adhesive 518 is sufficient to retain base stock 516 to carrier liner 520, yet permit base stock 516 and adhesive 518 to be released from carrier liner 520 when desired. In some embodiments, base stock 516, adhesive 518, and carrier liner 520 are rolled upon roll core 522 such that base stock 516 and adhesive 518 face toward roll core 522 to protect base stock 516 and adhesive 518 from contamination and reduce the possibility of premature release.

As can be seen in FIG. 59, in some embodiments, such a desired shape of carrier liner 520 can comprise a plurality of drive notches 524 formed along and slightly inboard of at least one of the elongated edges 526. The plurality of drive notches 524 can be shaped, sized, and spaced to permit cooperative engagement with a drive member to positively drive sealing cover roll 512 and aid in the proper positioning of sealing cover 80 relative to microplate 20. In the some embodiments, the desired shape of carrier liner 520 can further comprise a plurality of staging notches 528 to be used to permit reliable positioning of sealing cover 80. In some embodiments, the plurality of staging notches 528 can be formed along at least one elongated edge 526. In some embodiments, the plurality of staging notches 528 can be shaped and sized to permit detection by a detector, such as an optical detector, mechanical detector, or the like. An end/start of roll notch or other feature 530 can further be used in some embodiments to provide notification of a first and/or last sealing cover 80 on sealing cover roll 512. Similar to the plurality of staging notches 528, end/start of roll notch 530 can be shaped and sized to permit detection by a detector, such as an optical detector, mechanical detector, or the like. It should be appreciated that the foregoing notches and features can have other shapes than those set forth herein or illustrated in the attached figures. It should also be appreciated that other features, such as magnetic markers, non-destructive markers (e.g. optical and/or readable markers), or any other indicia may be used on carrier liner 520. To facilitate such detection with an optical detector to avoid physical contact, in some embodiments, carrier liner 520 can be opaque. However, in some embodiments, carrier liner 520 can be generally opaque only near elongated edges 526 with generally clear center sections 532 to aid in in-process adhesive inspection.

### Sealing Cover Applicator

In some embodiments, sealing cover 80 can be laminated onto microplate 20 using a hot roller apparatus 540, as illustrated in FIG. 29. In some embodiments, hot roller apparatus 540 comprises a heated top roller 542 heated by a heating element 544 and an unheated bottom roller 546. A first plate guide 548 can be provided for guiding microplate 20 into hot roller apparatus 540, while similarly a second plate guide 550 can be provided for guiding microplate 20 out of hot roller apparatus 540.

During sealing, sealing cover 80 can be placed on top of microplate 20 and the combination can be fed into hot roller apparatus 540 such that sealing cover 80 is in contact with first plate guide 548. As sealing cover 80 and microplate 20 pass and engage heated top roller 542, heat can be applied to sealing cover 80 to laminate sealing cover 80 to microplate 20. This laminated combination can then exit hot roller apparatus 540 as it passes second plate guide 550. In some embodiments, the heat from heated top roller 542 reduces the viscosity of the adhesive of sealing cover 80 to allow the adhesive to better adhere to microplate 20.

In some embodiments, hot roller apparatus 540 can variably control the amount of heat applied to sealing cover 80. In this regard, sufficient heat can be supplied to provide adhesive flow or softening of the adhesive of sealing cover 80 without damaging assay 1000. In some embodiments, hot roller apparatus 540 can variably control a drive speed of heated top roller 542 and unheated bottom roller 546. In some embodiments, hot roller apparatus 540 can variably control a clamping force between heated top roller 542 and unheated bottom roller 546. By varying these parameters, optimal sealing of sealing cover 80 to microplate 20 can be achieved with minimal negative effects to assay 1000.

### Sealing Liquid

In various some embodiments, microplate 20 can be covered with a sealing liquid prior to performance of analysis or reaction of assay 1000. In some embodiments, a sealing liquid can be a material that substantially covers the material retention regions (e.g., reaction spots, wells, reaction chambers) on microplate 20 to, at least in part, contain materials present in the material retention regions and reduce movement of material from one material retention region to another material retention region. In some embodiments, the sealing liquid can be any material that is not reactive with assay 1000 under normal storage or usage conditions. In some embodiments, the sealing liquid can be substantially immiscible with assay 1000. In some embodiments, the sealing liquid can be transparent, have a refractive index similar to glass, have low or no fluorescence, have a low viscosity, and/or be curable. In some embodiments, the sealing liquid can comprise a flowable, curable fluid such as a curable adhesive, such as, for example, ultra-violet-curable and other light-curable adhesives; heat, two-part, or moisture activated adhesives; and cyanoacrylate adhesives. In some embodiments, the sealing liquid can comprise mineral oil, silicone oil, fluorinated oils, and other fluids that are substantially immiscible with water.

In some embodiments, the sealing liquid can be a fluid when it is applied to the surface of the microplate and, in some embodiments, the sealing liquid can remain fluid throughout an analytical or chemical reaction using the microplate. In some embodiments, the sealing liquid can become a solid or semi-solid after it is applied to the surface of microplate 20.

### Thermocycler System

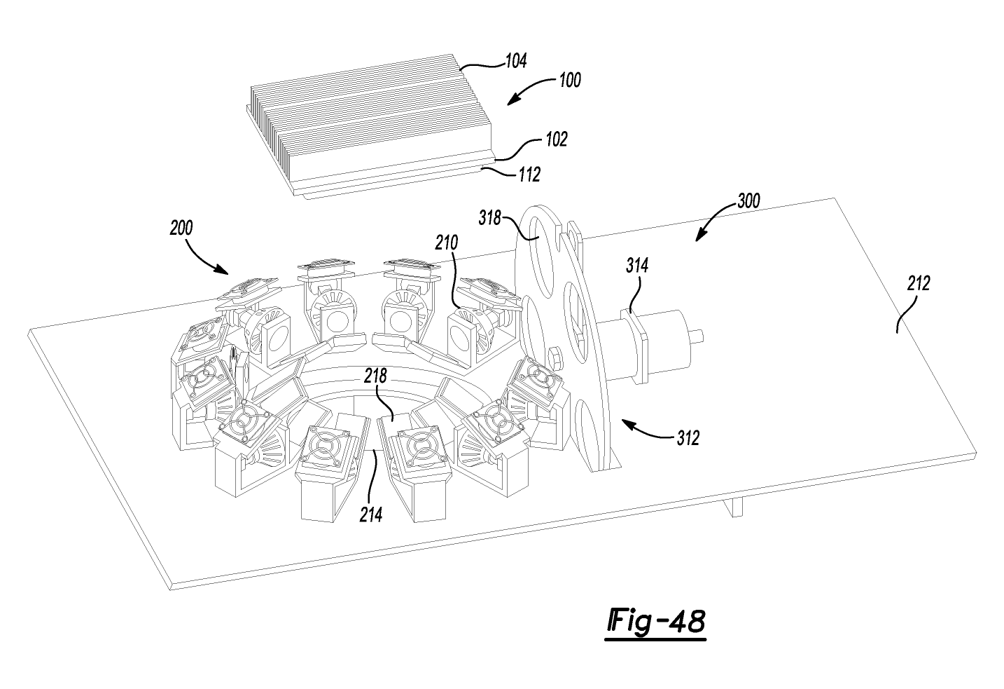

With reference to FIGS. 30-44, 47, and 48, in some embodiments, thermocycler system 100 comprises at least one thermocycler block 102. Thermocycler system 100 provides heat transfer between thermocycler block 102 and microplate 20 during analysis to vary the temperature of a sample to be processed. It should be appreciated that in some embodiments thermocycler block 102 can also provide thermal uniformity across microplate 20 to facilitate accurate and precise quantification of an amplification reaction. In some embodiments, a control system 1010 (FIGS. 30, 41, and 42) can be operably coupled to thermocycler block 102 to output a control signal to regulate a desired thermal output of thermocycler block 102. In some embodiments, the control signal of control system 1010 can be varied in response to an input from a temperature sensor (not illustrated).

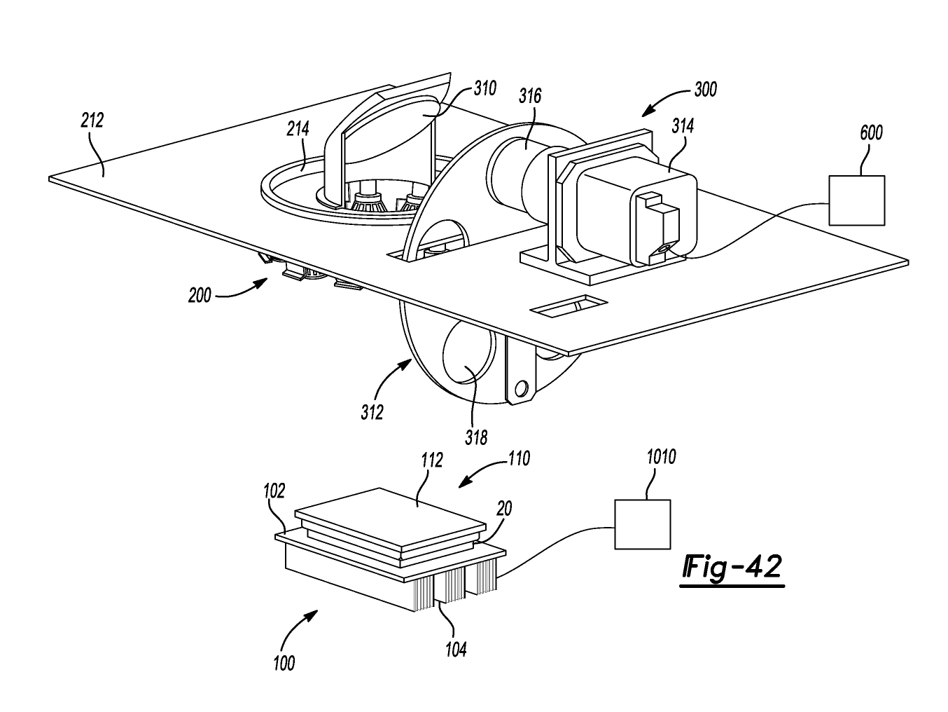

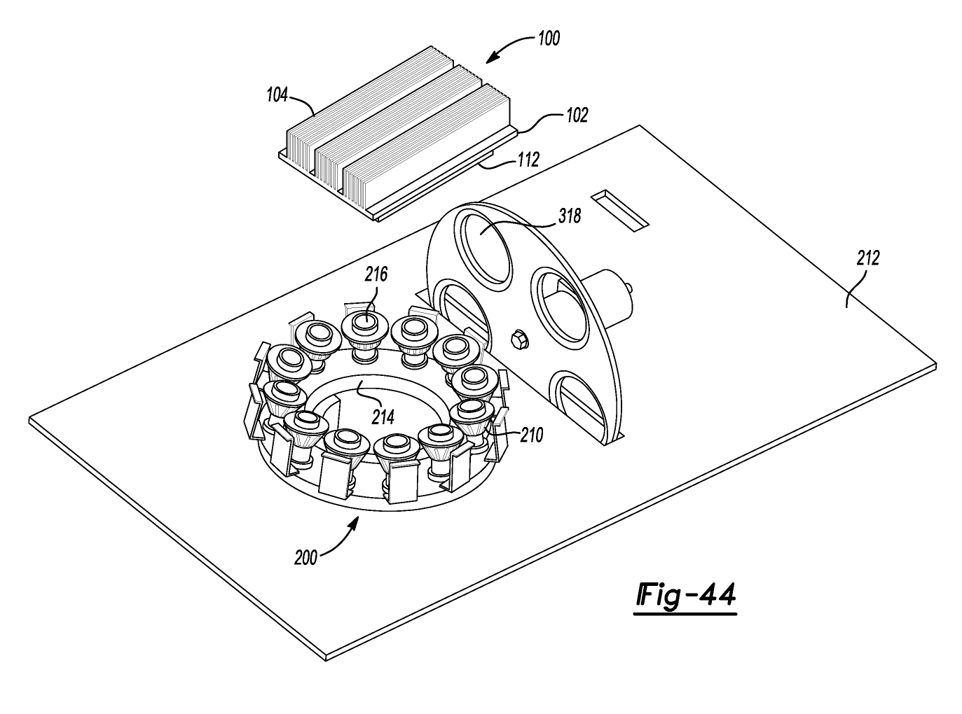

In some embodiments, thermocycler block 102 comprises a plurality of fin members 104 (FIGS. 42 and 44) disposed along a side thereof to dissipate heat. In some embodiments, thermocycler block 102 comprises at least one of a forced convection temperature system that blows hot and cool air onto microplate 20; a system for circulating heated and/or cooled gas or fluid through channels in microplate 20; a Peltier thermoelectric device; a refrigerator; a microwave heating device; an infrared heater; or any combination thereof. In some embodiments, thermocycler system 100 comprises a heating or cooling source in thermal connection with a heat sink. In some embodiments, the heat sink can be configured to be in thermal communication with microplate 20. In some embodiments, thermocycler block 102 continuously cycles the temperature of microplate 20. In some embodiments, thermocycler block 102 cycles and then holds the temperature for a predetermined amount of time. In some embodiments, thermocycler block 102 maintains a generally constant temperature for performing isothermal reactions upon or within microplate 20.

### Thermal Compliant Pad

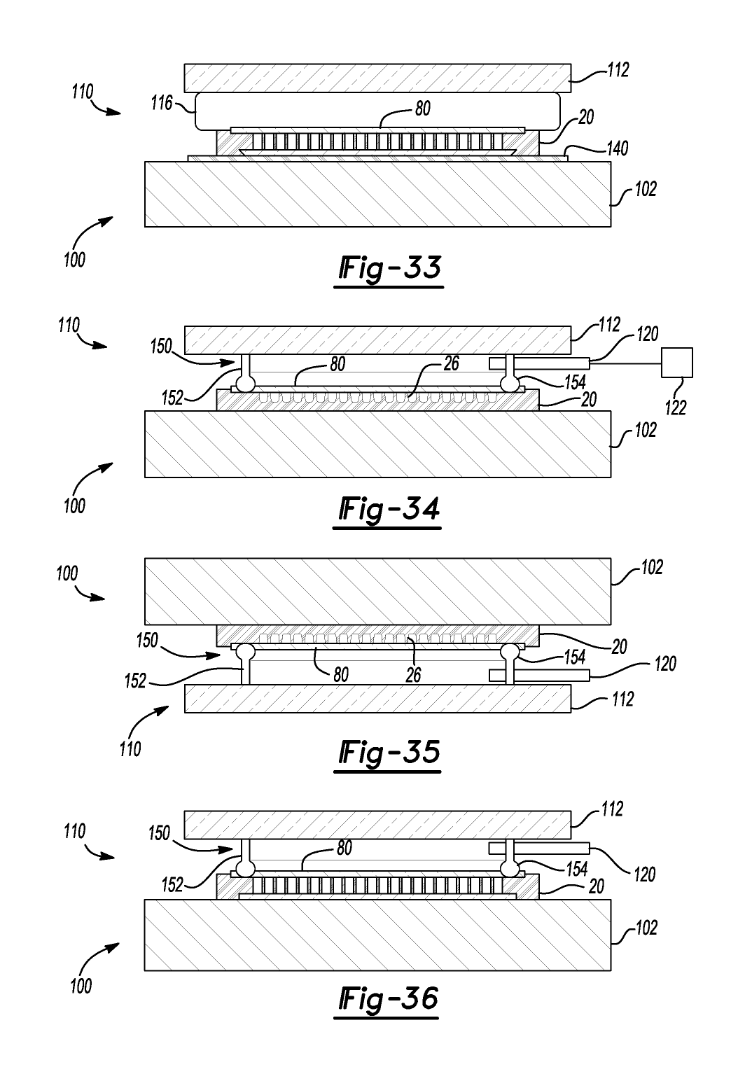

With reference to FIG. 33, thermal compliant pad 140 can be disposed between thermocycler block 102 and any adjacent component, such as microplate 20 or a sealing cover 80. It should be understood that thermal compliant pad 140 is optional. Thermal compliant pad 140 can better distribute heating or cooling through a contact interface between thermocycler block 102 and the adjacent component. This arrangement can reduce localized hot spots and compensate for surface variations in thermocycler block 102, thereby providing improved thermal distribution across microplate 20.

### Pressure Clamp System

As will be further described herein, according to some embodiments, pressure clamp system 110 can apply a clamping force upon sealing cover 80, microplate 20, and thermocycler block 102 to, at least in part, operably seal assay 1000 within the plurality of wells 26 during thermocycling and further improve thermal communication between microplate 20 and thermocycler block 102. Pressure clamp system 110 can be configured in any one of a number of orientations, such as described herein. Additionally, pressure clamp system 110 can comprise any one of a number of components depending upon the specific orientation used. Therefore, it should be understood that variations exist that are still regarded as being within the scope of the present teachings.

### Transparent Bag

As illustrated in FIGS. 30-33, in some embodiments, pressure clamp system 110 can comprise an inflatable transparent bag 116 positioned between and in engaging contact with a transparent window 112 and sealing cover 80. In the embodiment illustrated in FIG. 30, transparent window 112 and thermocycler block 102 are fixed in position against relative movement. Inflatable transparent bag 116 comprises an inflation/deflation port 118 that can be fluidly coupled to a pressure source 122, such as an air cylinder, which can be controllable in response to a control input from a user or control system 1010. It should be understood that in some embodiments inflatable transparent bag 116 can comprise a plurality of inflation/deflation ports to facilitate inflation/deflation thereof.

Upon actuation of pressure source 122, pressurized fluid, such as air, can be introduced into inflatable transparent bag 116, thereby inflating transparent bag 116 in order to exert a generally uniform force upon transparent window 112 and upon sealing cover 80 and microplate 20. In some embodiments, such generally uniform force can serve to provide a reliable and consistent sealing engagement between sealing cover 80 and microplate 20. This sealing engagement can substantially prevent water evaporation or contamination of assay 1000 during thermocycling. In some embodiments, inflatable transparent bag 116 can be part of the transparent window 112, thereby forming a bladder.

Still referring to FIG. 30, it should be appreciated that in some embodiments transparent window 112, inflatable transparent bag 116, and sealing cover 80 permit free transmission therethrough of an excitation light 202 generated by an excitation system 200 and the resultant fluorescence emission. Transparent window 112, inflatable transparent bag 116, and sealing cover 80 can be made of a material that is non-fluorescent or of low fluorescence. In some embodiments, transparent window 112 can be comprised of Vycor®, fused silica, quartz, high purity glass, or combination thereof. By way of non-limiting example, window 112 can be comprised of Schott Q2 quartz glass. In some embodiments, window 112 can be from about ¼ to about ½ inch thick; e.g., in some embodiments, about ⅜ inch thick. In some embodiments, a broadband anti-reflective coating can be applied to one or both sides of window 112 to reduce glare and reflections. In some embodiments, the transparent window 112 can comprise optical elements such as a lens, lenslets, and/or a holographic feature.

In some embodiments, as illustrated in FIG. 31, transparent window 112 can be movable to exert a generally uniform force upon transparent bag 116 and, additionally, upon sealing cover 80 and microplate 20. In some embodiments as in others, transparent bag 116 can comprise a fixed internal amount of fluid, such as air. Transparent window 112 can be movable using any moving mechanism (not illustrated), such as an electric drive, mechanical drive, hydraulic drive, or the like.

### Compressible Seal for Microplate

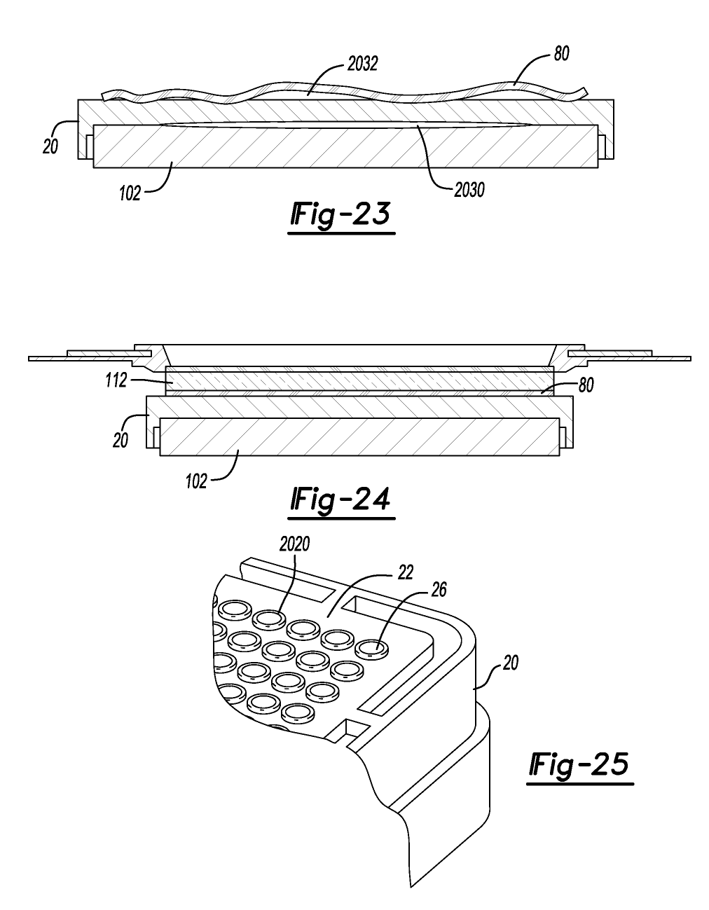

In some embodiments, as illustrated in FIG. 23, sealing cover 80 and/or microplate 20 may exhibit some variations in flatness, thereby resulting in some gaps existing between microplate 20 and thermocycler 102, which inhibit proper thermal contact, and/or sealing cover 80 and microplate 20, which can lead to contamination of assay 1000. In some embodiments as illustrated in FIG. 24, to overcome gaps formed between microplate 20 and sealing cover 80, sealing cover 80 can be made of a compliant material that can accommodate variations therebetween, yet maintain its transparency to permit transmission of excitation light 202 and/or fluorescence while minimizing its own flourescence. In some embodiments, sealing cover 80 can be made of a PDMS thin film membrane. This material can serve as both an optical cover and a compression pad that effectively seals wells 26 relative to each other.

In some embodiments, as illustrated in FIG. 25, microplate 20 can comprise a rim section 2020 disposed around each of the plurality of wells 26. If desired, rim section 2020 can be co-molded with microplate 20 to form an integral member extending upward from 22. Rim section 2020 can be made of a compliant material that is able to flex or otherwise conform to sealing cover 80 under pressure to define a sealing interface to prevent or at least inhibit cross-flow of assay 1000. In some embodiments, rim section 2020 can be made of polystyrene, which can plastically deform and further thermally fuse to sealing cover 80 under normal operating temperatures of sealing cover 80 (e.g. about 105° C.), which further ensures good well sealing.

These arrangements provide reduced costs, microplate sealing that can overcome microplate defects, and ease of installation.

### Pressure Chamber

In some embodiments, as illustrated in FIGS. 34-40, pressure clamp system 110 can further employ a pressure chamber 150 in place of transparent bag 116.

Pressure chamber 150 can be a pressurizable volume generally defined by transparent window 112, a frame 152 that can be coupled to transparent window 112, and a circumferential chamber seal 154 disposed along an edge of frame 152. Circumferential chamber seal 154 can be adapted to engage a surface to define the pressurizable, airtight, or at least low leakage, pressure chamber 150. Transparent window 112, frame 152, circumferential chamber seal 154, and the engaged surface bound the actual volume of pressure chamber 150. Circumferential chamber seal 154 can engage one of a number of surfaces that will be further discussed herein. A port 120, in fluid communication with pressure chamber 150 and pressure source 122, can provide fluid to pressure chamber 150.

In the interest of brevity, it should be appreciated that the particular configuration and arrangement of sealing cover 80 and microplate 20 illustrated in FIGS. 34-40 can be similar to that illustrated in FIGS. 30-33.

In some embodiments, as illustrated in FIGS. 34 and 36, circumferential chamber seal 154 can be positioned such that it engages a portion of sealing cover 80. A downward force from transparent window 112 can be exerted upon microplate 20 to maintain a proper thermal engagement between microplate 20 and thermocycler block 102. Additionally, such downward force can further facilitate sealing engagement of sealing cover 80 and microplate 20. Still further, pressure chamber 150 can then be pressurized to exert a generally uniform force upon sealing cover 80 and sealing interface 92. Such generally uniform force can provide a reliable and consistent sealing engagement between sealing cover 80 and microplate 20. This sealing engagement can reduce water evaporation or contamination of assay 1000 during thermocycling.

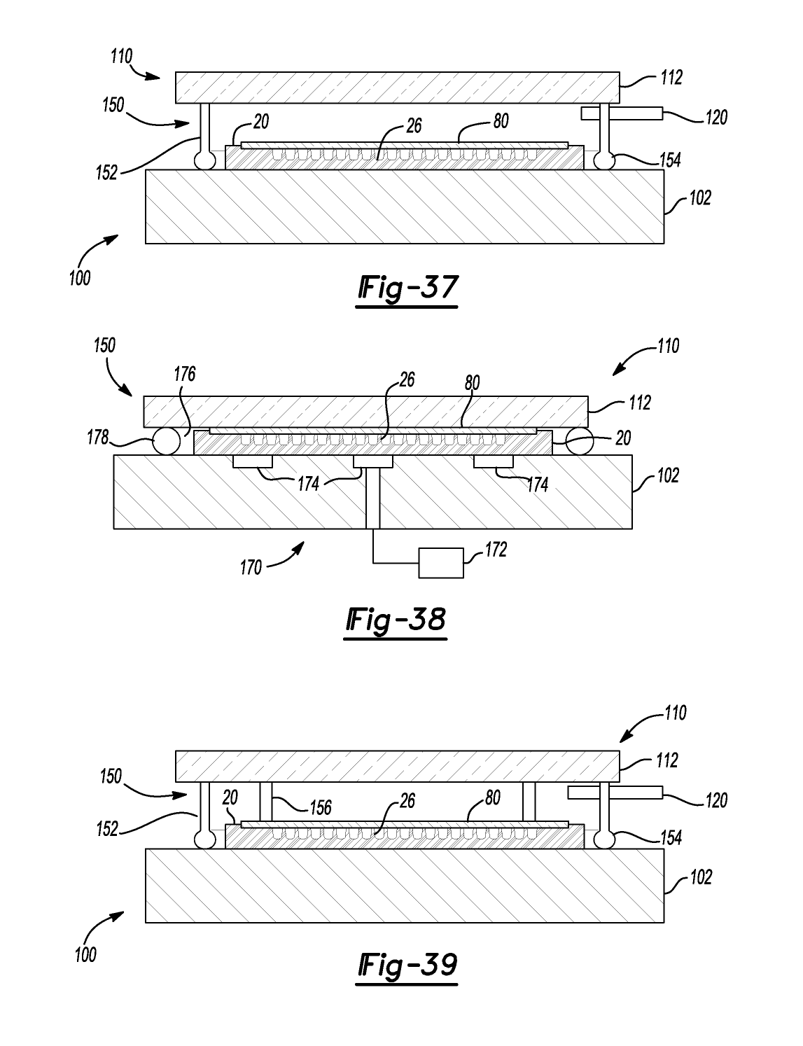

With particular reference to FIG. 37, it should be appreciated that in some embodiments circumferential chamber seal 154 of pressure chamber 150 can be positioned to engage thermocycler block 102, rather than microplate 20. Microplate 20 can be positioned within pressure chamber 150. As pressure chamber 150 is pressurized, force is exerted upon sealing cover 80, thereby providing a sealing engagement between sealing cover 80 and microplate 20.

In some embodiments, as illustrated in FIG. 39, to improve thermal contact between microplate 20 and thermocycler block 102, optional posts 156 can be employed. Optional posts 156 can be adapted to be coupled with transparent window 112 and downwardly extend therefrom. Optional posts 156 can then engage at least one of microplate 20 or sealing cover 80 to ensure proper contact between microplate 20 and thermocycler block 102 during thermocycling.

### Window Heating Device

In some embodiments, as illustrated in FIG. 41, transparent window 112 can comprise a heating device 160. Heating device 160 can be operable to heat transparent window 112, which in turn heats each of the plurality of wells 26 to reduce the formation of condensation within each of the plurality of wells 26. In some cases, condensation can reduce optical performance and, thus, reduce the efficiency and/or stability of fluorescence detection.

In some embodiments, heating device 160 can comprise a layer member 162 that can be laminated to transparent window 112. In some embodiments, layer member 162 can comprise a plurality of heating wires (not illustrated) distributed uniformly throughout layer member 162, which can each be operable to heat an adjacent area. In some embodiments, layer member 162 can be an indium tin oxide coating that is applied uniformly across transparent window 112. A pair of bus bars 164 can be disposed on opposing ends of transparent window 112. Electrical current can then be applied between bus bars 164 to heat the indium tin oxide coating, which provides a consistent and uniform heat across transparent window 112 without interfering with fluorescence transmission. Bus bars 164 can be controlled in response to control system 1010. In some embodiments, heating device 160 can be on both sides of transparent window 112.

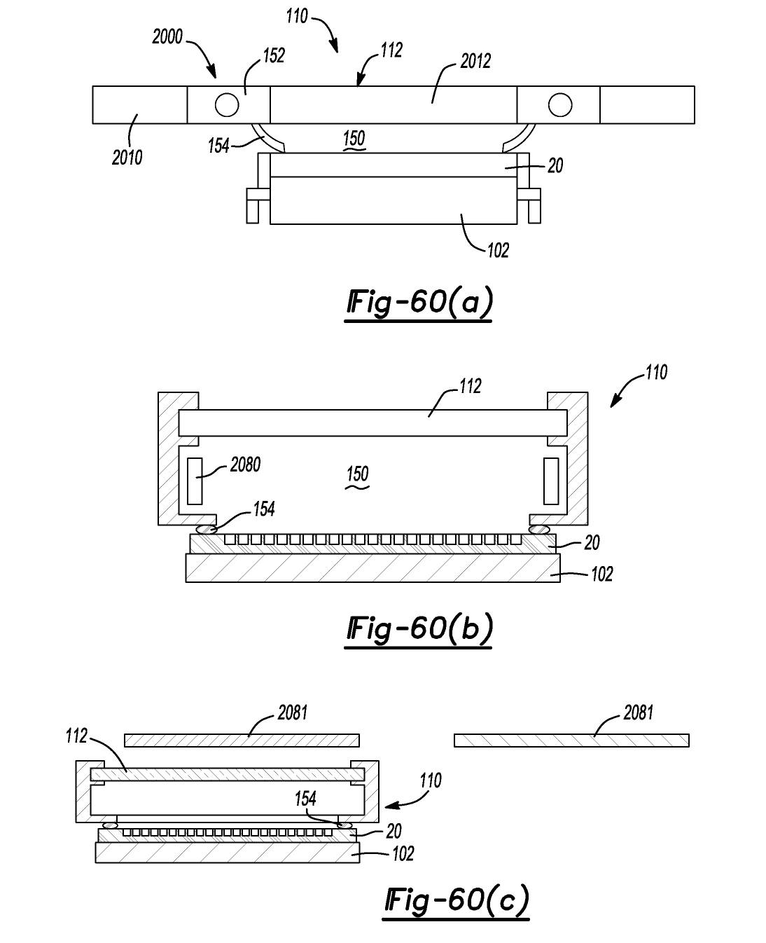

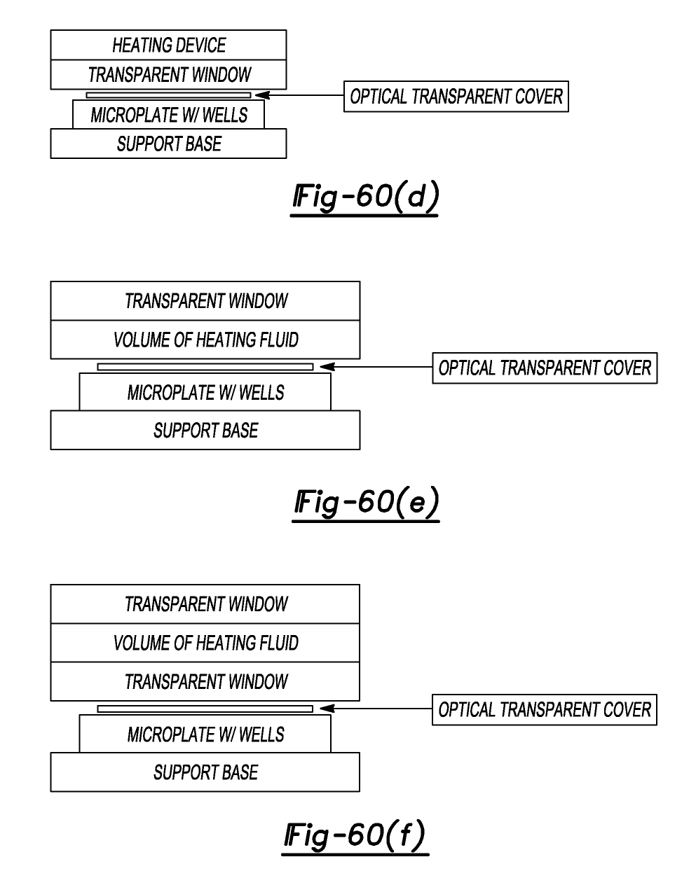

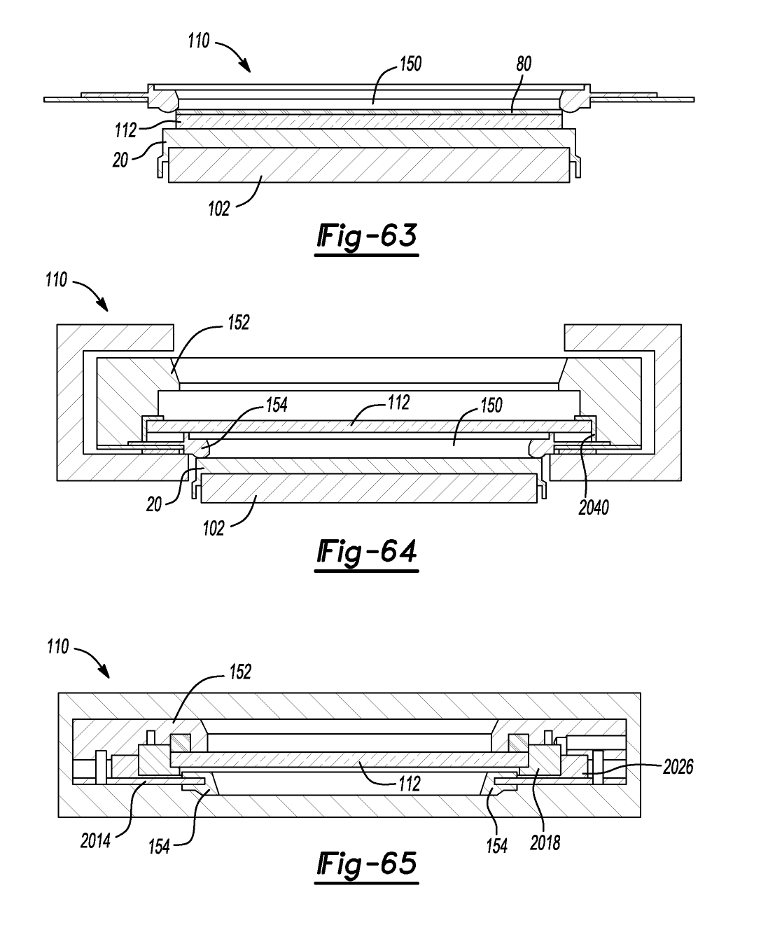

In some embodiments, as schematically illustrated in FIGS. 60(a)-67, pressure chamber 150 of pressure clamp system 110 can be a pressurizable volume generally defined by one or more of transparent window 112, a frame 152 that can be coupled to transparent window 112, and a circumferential or peripheral chamber seal 154 disposed along a portion of frame 152. Circumferential chamber seal 154 can be adapted to engage a surface, such as microplate 20, to define the pressurizable, airtight, or at least low leakage, pressure chamber 150. Additionally, in some embodiments, circumferential chamber seal 154 can serve as a thermal barrier to minimize heat transfer between frame 152 and microplate 20. To this end, a heater 2014 (see FIG. 65) can be added to circumferential chamber seal 154 to mitigate thermal edge effects due to contact of circumferential chamber seal 154 to microplate 20. In some embodiments, circumferential chamber seal 154 can also be made of a material that inhibits or minimizes heat transfer therethrough.

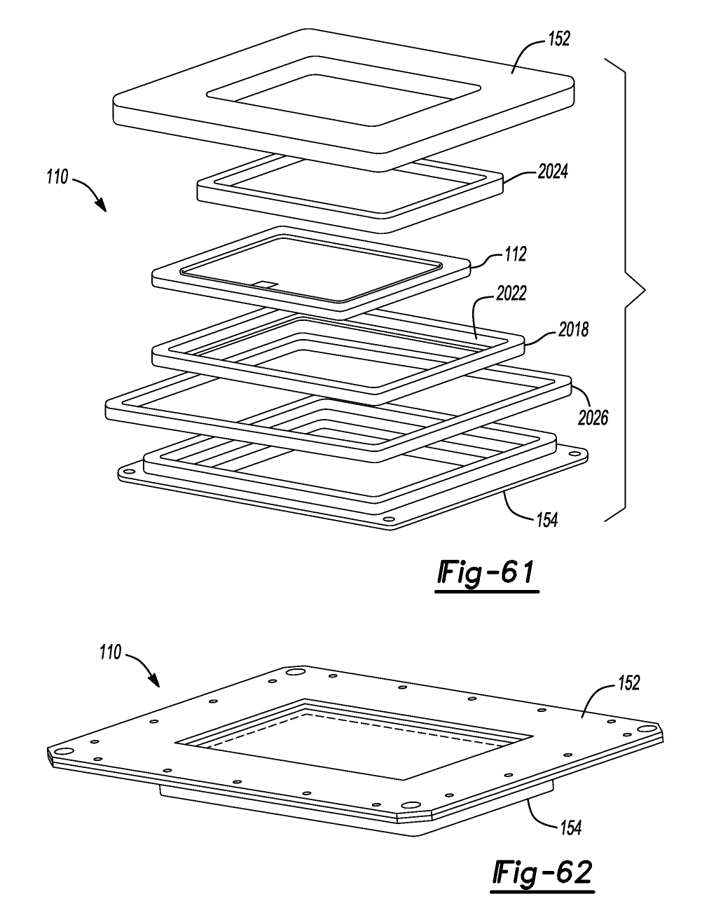

In some embodiments, as illustrated in FIGS. 61 and 62, pressure clamp system 110 can comprise a transparent window 112, a transparent window support 2018 having a relief portion 2022 sized to receive transparent window 112 therein. Transparent window support 2018 can be made of a strong, thermally-isolative material, such as PEEK or ULTEM. In some embodiments, as indicated herein, transparent window 112 can be heated. In some embodiments, as illustrated in FIG. 61, a gasket member 2024 can be disposed between transparent window 112 and frame 152 to provide, at least in part, thermal isolation between transparent window 112 and frame 152. Additionally, gasket member 2024 can provide a pressure seal between transparent window 112 and frame 152. Still referring to FIG. 61, a chamber body spacer 2026 can be disposed between transparent window support 2018 and circumferential chamber seal 154 to align and thermally isolate transparent window support 2018 and circumferential chamber seal 154. Such arrangement can, in some embodiments, permit the temperature of circumferential chamber seal 154 to be maintained independently from transparent window 112.

It should be understood that additional arrangements of pressure clamp system 110 can be used. For instance, as seen in FIGS. 24, 63, and 64, transparent window 112 can be positioned in contact with microplate 20 and/or sealing cover 80 or can be spaced apart therefrom.

### Pressure Aids Sealing Cover

In some embodiments, the pressure within pressure chamber 150 can aid in sealing each of the plurality of wells 26 by reliably forcing sealing cover 80 down thereon. In some embodiments, depending upon the size of the interstitial regions between adjacent wells 26, the adhesive used in a sealing cover may not adequate to maintain the seal integrity around each well 26 during heating when an internal vapor pressure within well 26 is produced. Therefore, the pressure within pressure chamber 150 can serve to combat this vapor pressure and maintain well integrity as seen in FIGS. 60, 64, and 67 and/or overcome any variations 2032 between sealing cover 80 and microplate 20 (see FIG. 23).

### Pressure Aids Microplate/Thermocycler Contact

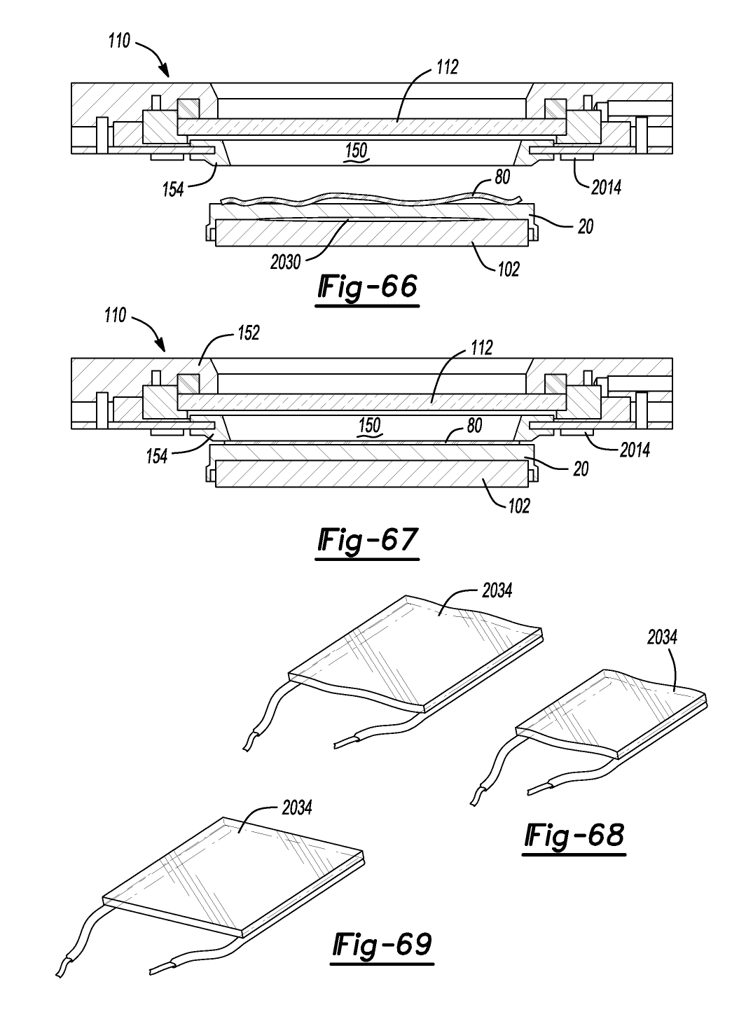

In some embodiments, the pressure within pressure chamber 150 can aid in maintaining proper thermal contact between microplate 20 and thermocycler block 102 by exerting a force upon microplate 20 and against thermocycler block 102. This force is constant across microplate 20, thereby causing high areas of microplate 20 into contact with thermocycler block 102, thereby reducing / substantially eliminating gaps 2030 therebetween (see FIGS. 23 and 66).

In some embodiments, a vacuum clamp may be used to augment or replace the pressure clamp to minimize/substantially eliminate gaps between the microplate and the thermocycler block. In some embodiments, a vacuum may be applied to at least a portion of the microplate thereby exerting a force on the microplate. The force provided by the vacuum may assist in pulling the microplate onto a support base or the thermocycler block. In some embodiments, an optical cover seal is provided to seal the wells of the microplate such that the optical cover seal provides a sufficient barrier that can withstand the vapor pressure in the wells while the vacuum is applied. The vacuum may further be applied to at least a portion of the bottom of the microplate directly. Alternatively, the vacuum may evacuate the chamber 150 in such a manner so as to exert a force on the microplate that acts to secure the plate in a desired position.

### Heat Minimizes Condensation

In some embodiments, transparent window 112 can be heated and/or cooled to aid in heat cycling assay 1000 during a PCR process. This heating can, at least in part, prevent or at least minimize condensation that might otherwise form on circumferential chamber seal 154, transparent window 112, and other portions of pressure chamber 150, which can adversely affect the PCR reaction as well as inhibit the optical transmission to detection system 300. To this end, in some embodiments, a heater system 2000 can be employed to heat and/or cool at least a portion of transparent window 112. Additionally, in some embodiments where an unobstructed line of sight into well 26 is needed, such as during real-time PCR, heater system 2000 can comprise a high conductivity portion for improved heating of microplate 20, which will be described in greater detail herein.

With continued reference to FIG. 60(a), in some embodiments, transparent window 112 can comprise a multi-portion system having one or more of frame 152, heater system 2000, a low conductivity portion 2010 (in some embodiments, also known as gasket member 2024 and/or frame 152), and/or a high conductivity portion 2012 (in some embodiments, also known as transparent window 112). In some embodiments, high conductivity portion 2012 can be transparent to provide an unobstructed line of sight into the plurality of wells 26 of microplate 20. In some embodiments, high conductivity portion 2012 is strong and thermally conductive. In some embodiments, high conductivity portion 2012 is a sapphire crystalline window, which is transparent, synthetic-sapphire, comparable to aluminum in strength and scratch resistant, and relatively conductive (about 30 times more thermally conductive than fused silica windows). In some embodiments, high conductivity portion 2012 or transparent window 112 can comprise a sapphire crystalline material, sapphire crystal layers, sapphire compositions, diamond crystal layers, diamond compositions, and other heat conductive crystalline materials that provide a sufficient degree of optical clarity. These materials can be provided as solid members or thin films. Sapphire crystalline windows or crystals can be obtained from RAYOTEK SCIENTIFIC INC. (San Diego, Calif.) or SWISS JEWEL COMPANY (Philadelphia, Pa.).

To provide heat to high conductivity portion 2012, heater system 2000 can output heat, which can then be evenly conducted through high conductivity portion 2012. Heater system 2000 can include any one of a number of heaters, such as but not limited to strip heaters, resistive heaters, cast-in heater, and the like. The heat contained in high conductivity portion 2012 can be transferred to microplate 20 via convention and/or conduction. More particularly, the heat in high conductivity portion 2012 can be conducted through the air or other gas contained in pressure chamber 150 to provide thermal communication between heater system 2000 and assay 1000 contained in the plurality of wells 26 of microplate 20. In fact, the pressure within pressure chamber 150 can be varied to control the thermal communication between high conductivity portion 2012 and microplate 20—that is, more pressure provides more thermal communication and likewise less pressure provides less thermal communication. Moreover, the heat being conducted through high conductivity portion 2012 and the air within pressure chamber 150 can serve to prevent or at least minimize any condensation forming on or near wells 26, including any sealing cover used.

In some embodiments, depending on the number of wells 26 disposed in microplate 20 and particularly the area formed between adjacent wells 26, a heating element, such as a resistive heater, can be disposed in the interstitial regions between adjacent wells 26. However, in some embodiments having higher well densities, this region may be too small to accommodate a resistive heater; thereby other heater systems may be used.

### Indium Tin Oxide (ITO) Thin Film Heater

In some embodiments, as illustrated in FIGS. 68-70, heater system 2000 can comprise an indium tin oxide (ITO) thin film heater 2034 operable to heat high conductivity portion 2012. In some embodiments, a thin film of transparent resistive conductive material is deposited on high conductivity portion 2012. This provides, at least in part, a uniform power output on the surface of high conductivity portion 2012 and thus can generate heat on the top of sealing cover 80 and microplate 20 through free convection and radiation. Power output can be on the order of 50 W and is sufficient to remove condensation that develops on microplate 20 and/or sealing cover 80. This indium tin oxide thin film heater can be purchased from JDS UNIPHASE. In some embodiments, a secondary heater could be employed to heat air being introduced into pressure chamber 150.

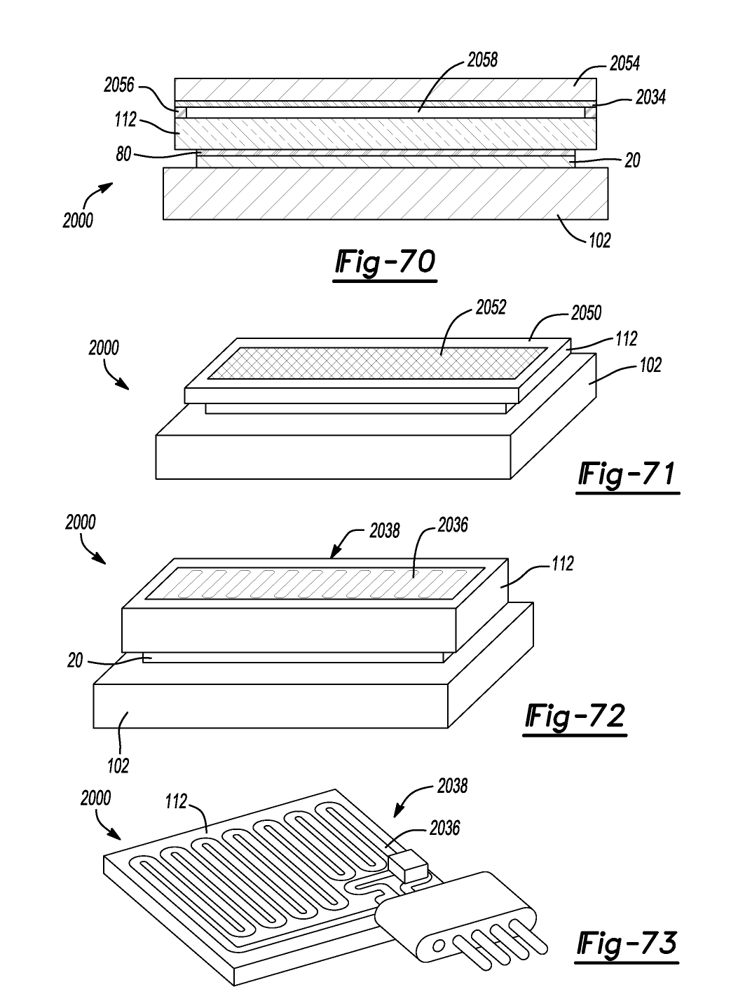

As in some embodiments, as illustrated in FIG. 70, heater system 2000 can comprise a transparent hot plate 2054 disposed on an opposing side of transparent window 112. Transparent hot plate 2054 can include a transparent resistive thin film (ITO) 2056 operable to heat transparent hot plate 2054. Additionally, in some embodiments, transparent hot plate 2054 and/or transparent resistive thin film (ITO) 2056 and can be spaced apart from transparent window 112 to form a gap 2058 therebetween (FIG. 70).

In some embodiments, heat transmission may be enhanced by the inclusion of one more layers of antireflective coatings/materials that have appropriate index matching characteristics for the particular ITO design. The antireflective coatings/materials may substantially preserve the uniform optical transmission capability of the ITO. Inclusion of the antireflective coatings with the indium tin oxide layer may further improve/modify the optical characteristics and/or heat transmission characteristics in a desirable manner.

In some embodiments the heated cover includes a chamber with a transparent window having internally positioned heaters. In some embodiments, the heaters are embedded within the window and positioned during molding of the window. Additionally, the heaters may be positioned within channels or pockets formed within the window. The channels of pockets may be molded into the window during its fabrication or subsequently formed by chemical or mechanical methods including by way of example, etching, routing drilling. As with other embodiments the heaters may be composed of thin wires, sputter deposited, lithographically deposited, vapor deposited, thin layer coated, or other known methods for providing for the conductive elements of the heater.

With reference to FIG. 60(b) an alternative embodiment of a heated cover design is shown. In some embodiments heaters are positioned in such a manner so as to heat the gaseous content/atmosphere inside the chamber 150. The heating of the chamber 150 contents is sufficient such that the heated gaseous content of the chamber 150 heats the window 112. In some embodiments, the transparent window 112 is heated by air heaters 2080 inside the chamber and the plate seal 154 is heated by radiation from the surface of the transparent window 112, and by conduction and convection of hot air in the chamber 150.

With reference to FIG. 60(c) an alternative embodiment of a heated cover design is shown. In some embodiments, a shuttle 2081 is provided with the pressure chamber and transparent window. In an exemplary application, the shuttle may be used to shuttle the heated plate between a first (for example heating) position and second (for example read) position. In the heating position, the shuttle 2081 may be configured to reside substantially above or in close proximity to the transparent window 112. Various heating sources including IR radiation, conduction, and convection may transfer heat from the shuttle to the window and reaction plate.

In some embodiments, the heated plate can be operated at substantially increased temperatures (for example 150-250 deg C.) to produce adequate IR heating to heat the seal. The heated plate may further be moved to the read position providing optical access to the plate. The window may further be constructed of a material with high IR transmission efficiency. Coatings on the window may further be designed to minimize IR reflection.

With reference to FIG. 60(d) an alternative embodiment of a heated cover design is shown. In some embodiments, a substantially direct contact approach is used for transferring heat from the heated cover to the plate. This embodiment may be adapted for use with open well plate formats by the inclusion of an additional cover interposed between the open wells of the plate and the heated cover. In some embodiments, the additional cover may directly contact the heated cover effectuating heat transfer between the heated cover and the additional cover. In other embodiments, including some embodiments wherein the plate format is a closed well configuration (such as TLDA cards) the additional cover is not used.

With reference to FIGS. 60(e) and 60(f) alternative embodiments of heated cover designs are shown. According to these embodiments, one or more optical covers may be used in the clamp design. For example, as shown in FIG. 60(e), a single optical cover may be used to create a chamber in which heated fluid or gas is contained or passed. The chamber or gap shown in this approach may be configured as a pressure chamber to apply a desired clamping force sufficient either isolate or seal the wells of the microplate by exerting a force on the plate cover. Additionally, the pressure clamp may exert sufficient pressure to secure the plate against the support base or thermocycler block. Various heating methods described elsewhere may be adapted to this architecture of clamping. Additionally, the fluid or gas contained in the chamber may be pressurized. Further, the chamber may be adapted with inlet and outlets to permit flow of the gas or fluid with a desired velocity through the chamber.

As shown in FIG. 60(f), additional transparent windows may be used in the clamp design. The transparent windows may be used to isolate the chamber or gap from the reaction plate. In some embodiments, this configuration permits substantially direct contact between the reaction plate and at least one of the transparent windows whereby heat may be conducted from the chamber or gap through the at least one optical cover to heat the reaction plate.

As described elsewhere, a vacuum may further be configured to secure the plate in a desired position. For example, In FIG. 60(f) a vacuum may be applied to a portion of the plate substantially simultaneously with the optical cover being in direct contact with the reaction plate.

In various embodiments, the transparent windows may apply a mechanical force to secure the plate. In such embodiments, the gap or chamber of the clamp need not be pressured for purposes of securing the plate. In various embodiments, the gap or chamber may still provide desired heating of the plate while a mechanical force is used to secure the plate, clamp, or portions thereof.

### Thin Wire Heater

In some embodiments, as illustrated in FIGS. 72 and 73, heater system 2000 can comprise a thin wire heater 2036 made of a heater material/element, such as gold, that is deposited directly onto transparent window 112 in the form of a heater circuit 2038. The circuit can be directly bonded on high conductivity portion 2012 along a perimeter thereof such that the heater element is outside the field of view or prescribed clear aperture or such that it permits excitation light 202 to pass therethrough and detection of the resultant flourescence. This bonding method can enhance robustness because it is easier to ensure that there is good thermal contact between high conductivity portion 2012 and the heater element thus reducing the risk of a heater failure. Due to the nature of high conductivity portion 2012, such is the case with sapphire crystal layers, sapphire compositions, diamond crystal layers, diamond compositions, and other heat conductive materials that provide a sufficient degree of optical clarity, a perimeter heater can provide sufficient heat at the edges in order to heat the entire high conductivity portion 2012 with acceptable non-uniformities there across. This thin wire heater can be purchased from NOVEL CONCEPTS INC.

### Simple Resistive Heater

In some embodiments, as illustrated in FIG. 64, heater system 2000 can comprise a perimeter heater 2040, such as capton or silicone rubber heaters, fastened with pressure sensitive adhesive to high conductivity portion 2012. The perimeter heating element 2040 can be placed along a perimeter of high conductivity portion 2012 such that the perimeter heater is outside the field of view or prescribed clear aperture. Such arrangement provides an economical and simple installation solution to applying heat to high conductivity portion 2012.

In some embodiments, as illustrated in FIG. 71, heater system 2000 can comprise a metal heated cover 2050 that is placed adjacent high conductivity portion 2012 in an overlapping relationship. Metal heated cover 2050 can comprise a plurality of through holes 2052 formed therein to permit excitation light 202 therethrough to excite one or more components of assay 1000 and/or detection of any resultant fluorescence therefrom. In some embodiments, metal heated cover 2050 could be formed using a thin resistive metal deposition or a stamped resistive pattern.

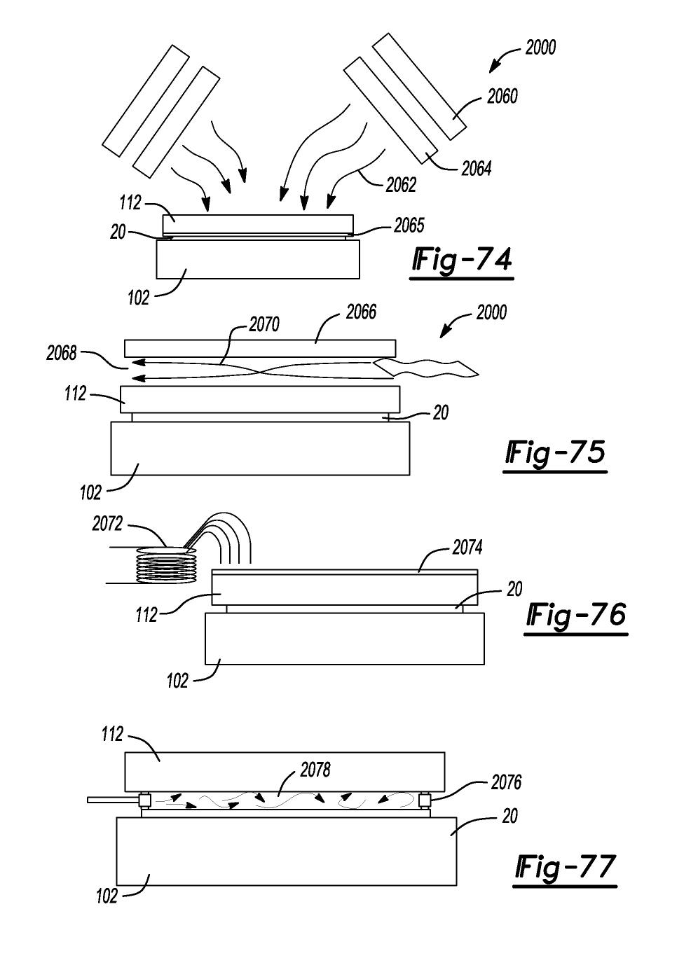

In some embodiments, as illustrated in FIG. 74, heater system 2000 can comprise infrared (IR) heaters 2060 emitting infrared energy 2062 to heat transparent window 112. In some embodiments, transparent window 112 can comprise an infrared absorbing layer 2065 operable to readily produce heat in response to infrared energy 2062. A diffuser 2064 can be used to provide a more uniform distribution of energy to transparent window 112.

In some embodiments, an infrared heating mechanism may be adapted to heat the optical cover more directly. For example, an IR transmitting source or material may be included in the cover. ITO as described in various embodiments may be configured to heat at least partially by this mechanism. Furthermore, other materials/compositions may be adapted to provide a desired IR transmission source that may be formed as a layer to reside in proximity to the reaction plate thereby heating the plate substantially directly.

In some embodiments, as illustrated in FIG. 75, heater system 2000 can comprise a second transparent window 2066 that is spaced apart from an opposing side of transparent window 112 to form a volume 2068. Volume 2068 is sized to receive a convective fluid or gas 2070 therein for heating transparent window 112. In this arrangement, transparent window 112 and consequently assay 1000 and microplate 20 could be heated and cooled more quickly due to the efficiency of the convective fluid 2070, if desired. In various embodiments, the convective fluid or gas 2070 may further be pressurized to a desired amount. Pressurization of the convective fluid or gas 2070 may serve as a mechanism by which to secure the plate in a desired position and/or to reduce or substantially eliminate gaps between the reaction plate and thermocycler block.

In some embodiments, the convective fluid or gas 2070 is configured to flow with a selected velocity or rate. The velocity or rate of flow may be configured to regulate the amount of heat transferred to the reaction plate. Furthermore, the velocity or rate of flow of the convective fluid or gas may be configured to attain a desired rate of exchange of the fluid or gas within the clamp with respect to an external reservoir or transport apparatus (for example a pump).

In some embodiments, as illustrated in FIG. 76, heater system 2000 can comprise an induction heater 2072 operably coupled to a transparent conductive layer 2074 mounted on transparent window 112. In this way, induction heater 2072 outputs heat to transparent conductive layer 2074 that heats transparent window 112, thereby heating microplate 20 and assay 1000. In some embodiments, transparent conductive layer 2074 could be made to distribute heat extremely fast and/or in a given pattern to accommodate any variation in transparent window 112, microplate 20, and/or other environmental effects.

In some embodiments, as illustrated in FIG. 77, heater system 2000 can comprise a seal 2076 engaging microplate 20 or other surface to form a chamber 2078. A hot/cold air, gas, or fluid can be introduced into chamber 2078 to heat/cool microplate 20 and assay 1000. The hot/cold air, gas, or fluid is particularly useful in maintaining a desired temperature. Additionally, turbulent mixing can aid in heat transfer to microplate 20 and assay 1000 and further aid in providing uniform temperatures across microplate 20.

### Diamond Thin Films

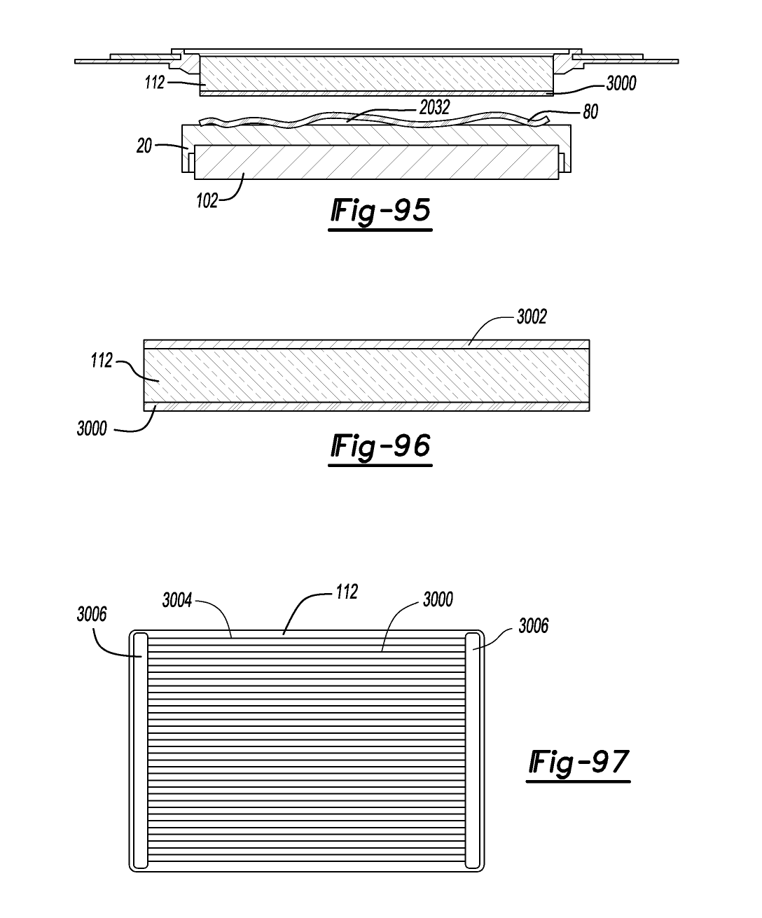

In some embodiments, as illustrated in FIGS. 95-99, transparent window 112 can comprise a diamond thin film 3000 coupled thereto to, at least in part, provide an extremely hard surface that protects transparent window 112, distribute heat across the surface of transparent window 112, and function as a possible heat source.

Diamond thin film 3000 can be grown or otherwise deposited upon transparent window 112 using microwave plasma CVD processes, according to processes taught and sold by KOBE STEEL, LTD. and ADVANCED DIAMOND TECHNOLOGIES, which developed UNCD® (ultrananocrystalline diamond) utilizing a patented processes for fabricating and tuning the properties of the films. Due to diamond's extreme hardness, diamond thin film 3000 is well suited for these types of protective applications to protect transparent window 112. Furthermore, diamond thin film 3000 further provides a highly-desired optically clear system that is resistant to scratches and other scattering effects. Furthermore, another property of diamond thin film 3000 that is particular conducive to the present application includes its high heat conductivity and its qualities as a heat sink and/or heat spreader.

In some embodiments, diamond thin film 3000 can be used as a heating device. Although natural diamonds are typically electrical insulators, the addition of dopants, in connection with the present teachings, can cause diamond thin film 3000 to become electrically conductive, thus enabling the potential for use as a resistive heater. Additionally, in some embodiments, diamond thin film 3000 can be electrically insulated as an un-doped diamond film.

With particular reference to FIGS. 95 and 96, in some embodiments, diamond thin layer 3000 can be applied to a bottom surface of transparent window 112. In such a manner, transparent window 112 is protected from such scratches or abrasions and further provides uniform thermal distribution. As seen in FIG. 96, a window heater 3002, similar to those described herein under differing reference numerals, can be used to apply a thermal load to transparent window 112 along a side opposite that of diamond thin film 3000.

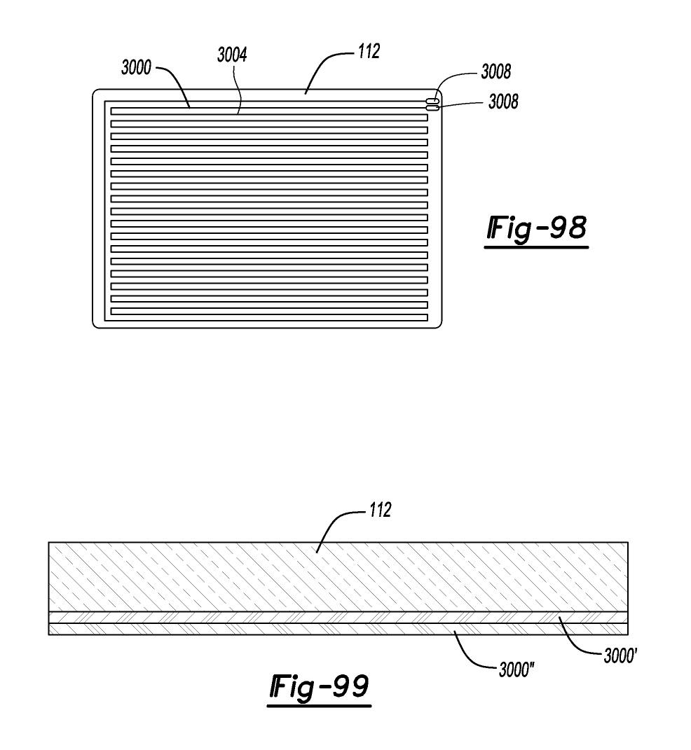

As seen in FIGS. 97 and 98, diamond thin layer 3000 can be patterned following deposition to include resistive paths 3004 for application of resistive heat. These resistive paths 3004 can take various configurations such as narrow parallel lines to form resistive heating elements collectively coupled on opposing ends busses 3006 (FIG. 97). Busses 3006 can then be coupled to a power source for application of electrical power to generate such resistive heat. It should be understood that an additional layer of diamond thin film can be applied over the resistive paths so as to provide a protective barrier. Additionally, the resistive paths 3004 can be patterned as a continuous line forming a single circuit path terminating at contact ends 3008. However, it should be appreciated that there can be additional circuit paths, if desired. As illustrated in FIG. 99, diamond thin layer 3000 having a resistive path 3004 (hereinafter 3000′) can include an additional diamond thin layer 3000 (hereinafter 3000″) disposed there over for protection of diamond thin layer 3000′ from shorts and to make the structure highly durable.

### Gap Size Selection

It should be understood that high conductivity portion 2012, when heated or cooled, can provide a laterally uniform heating element to provide substantially uniform heating of assay 1000 in microplate 20. Some embodiments that can further aid in producing this uniformity is the size of the air gap defined by pressure chamber 150. That is, by selecting a proper distance, this heating and cooling uniformity can be maximized through convection and conduction properties. For example, if the air gap is too large, there may be insufficient thermal communication between high conductivity portion 2012 and microplate 20, thus allowing condensation to form. If the air gap is too small, the heating may be non-uniform, which in turn may cause non-uniform heating of assay 1000 in microplate 20, leading to variation in the resultant data. However, it should be understood that the optimal air gap distance is dependent upon the particular heater system 2000 used, the environmental conditions, the effect on assay 1000 of microplate 20, and the like.

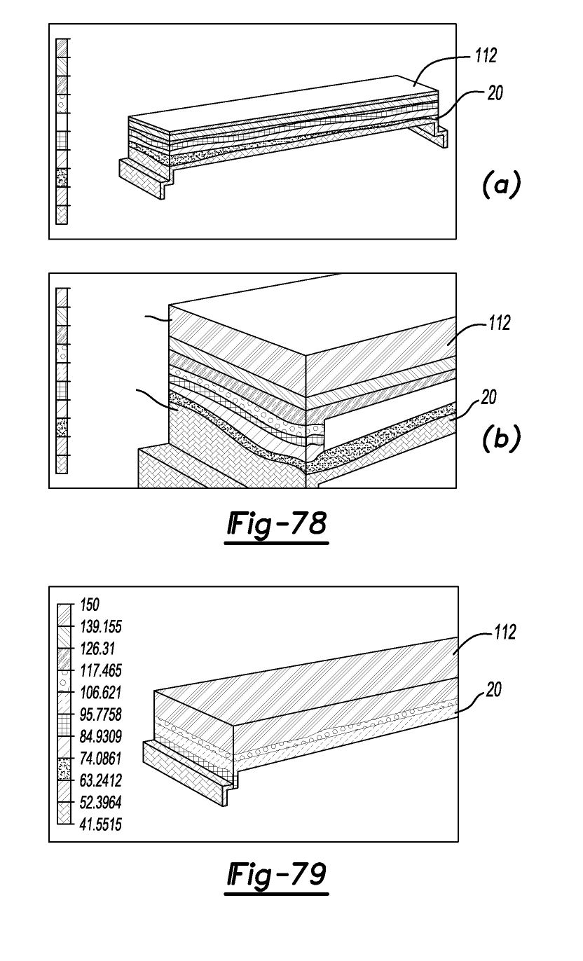

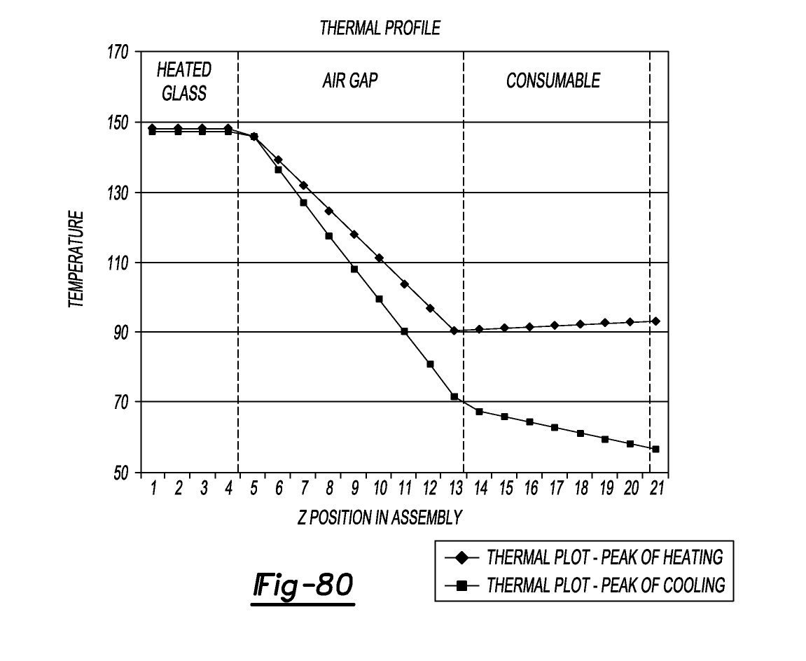

In some embodiments, one potential heat transfer mechanism arises from radiative heat transferred between the window 112 and the seal 80. In such instances, an increase in the temperature of the heated window 112 may result in an increase in temperature of the seal surface 80. It will be appreciated by one of skill in the art that the efficiency of this thermal transfer may depend on various factors including, among others, the material composition of the window 112 and seal 80, the distance between the window 112 and seal 80, and air flow between the window 112 and the seal 80. With particular reference to FIGS. 78-80, it can be seen that at the peak of a cooling cycle (about 60° C. in a PCR cycle, FIGS. 78(a)-(b)), transparent window 112 remains uniform in temperature across its face and the air in the air gap aids in maintaining this uniformity. Similarly, it can be seen that at the peak of a heating cycle (about 95° C. in a PCR cycle, FIG. 79), transparent window 112 again remains uniform in temperature across its face and the air in the air gap aids in maintaining this uniformity. It should be noted that in FIGS. 71(a), 78(b), and 79, temperature gradients are illustrated and thus the noted striations are indicative of uniform temperatures and not material cross sections. Finally, as seen in the graph of FIG. 80, the temperature gradient as a function of Z position is illustrated such that a smooth variation occurs with position between the heating cycle and the cooling cycle.

### Inverted Orientation

In some embodiments, as illustrated in FIGS. 27, 32, 35, 41, 44, 47, and 48, microplate 20 can be inverted such that each of the plurality of wells 26 is generally inverted, such that the opening of each of the plurality of wells 26 is directed downwardly. Among other things, this arrangement can provide improved fluorescence detection. As illustrated in FIG. 27, this inverted arrangement causes assay 1000 to collect adjacent sealing cover 80 and, thus, addresses the occurrence of condensation effecting fluorescence detection and improves optical efficiency, because assay 1000 is now disposed adjacent to the opening of each of the plurality of wells 26.

In some embodiments, as illustrated in FIG. 32, thermocycler block 102 remains stationary and is positioned above microplate 20 and transparent window 112 is positioned below microplate 20. Inflatable transparent bag 116 can then be positioned in engaging contact between transparent window 112 and sealing cover 80. It should be appreciated that transparent window 112, inflatable transparent bag 116, and sealing cover 80 can permit free transmission therethrough of excitation light 202 generated by excitation system 200 positioned below transparent window 112 and the resultant fluorescence therefrom. In some embodiments, detection system 300 can be positioned below microplate 20 to detect such fluorescence generated in response to excitation light 202 of excitation system 200.

In some embodiments, as illustrated in FIG. 35, microplate 20 can be positioned in an inverted orientation, similar to that described in connection with FIG. 32, and further employ pressure chamber 150. Circumferential chamber seal 154 can then be positioned such that it engages a portion of sealing cover 80. A force from transparent window 112 can be exerted upon microplate 20 to maintain a proper thermal engagement between microplate 20 and thermocycler block 102 and sealing engagement between sealing cover 80 and microplate 20. Pressure chamber 150 can then be pressurized to exert a generally uniform force across sealing cover 80.

### Relief Port

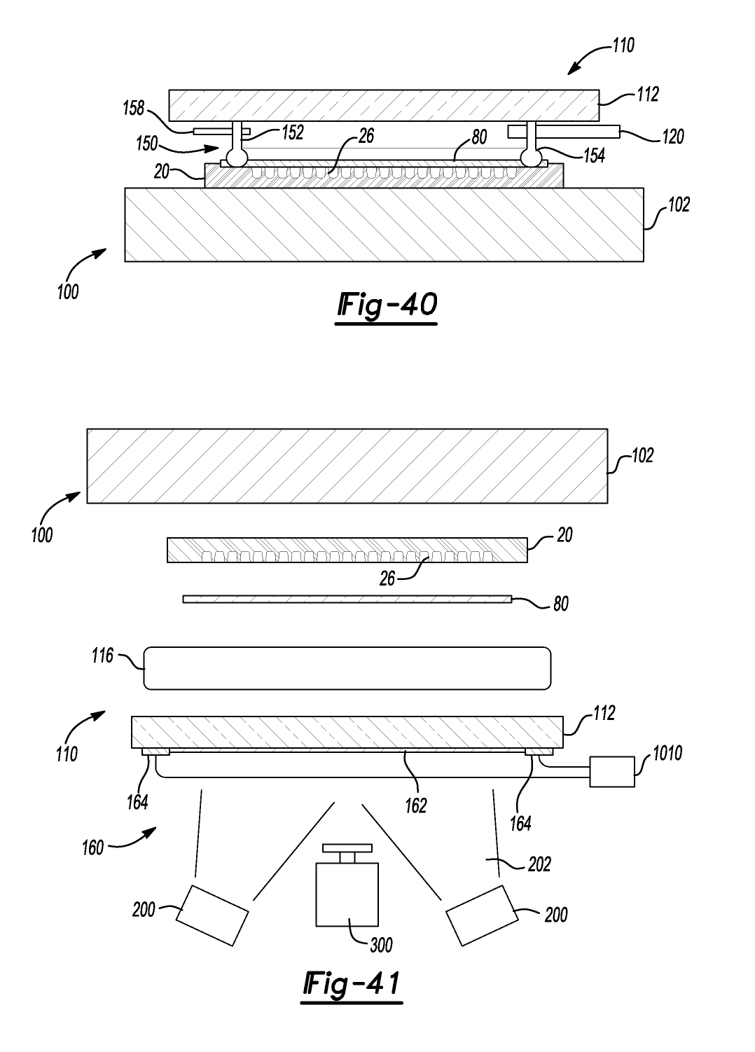

Turning now to FIG. 40, in some embodiments a relief port 158 can be in fluid communication with pressure chamber 150. Relief port 158 can be operable to slowly bleed gas in pressure chamber 150 and/or simultaneously remove water vapor from pressure chamber 150 to reduce condensation. Removal of water vapor can, in some circumstances, improve fluorescence detection. Relief port 158 can be used in connection with any of the embodiments described herein.

### Clamp Mechanism

In some embodiments, as seen in FIGS. 84-88, pressure chamber 150 can be used with a clamp mechanism 1400 (best illustrated in FIGS. 86-88). Clamp mechanism 1400 can retain pressure chamber 150 in a clamped position against thermocycler system 100.

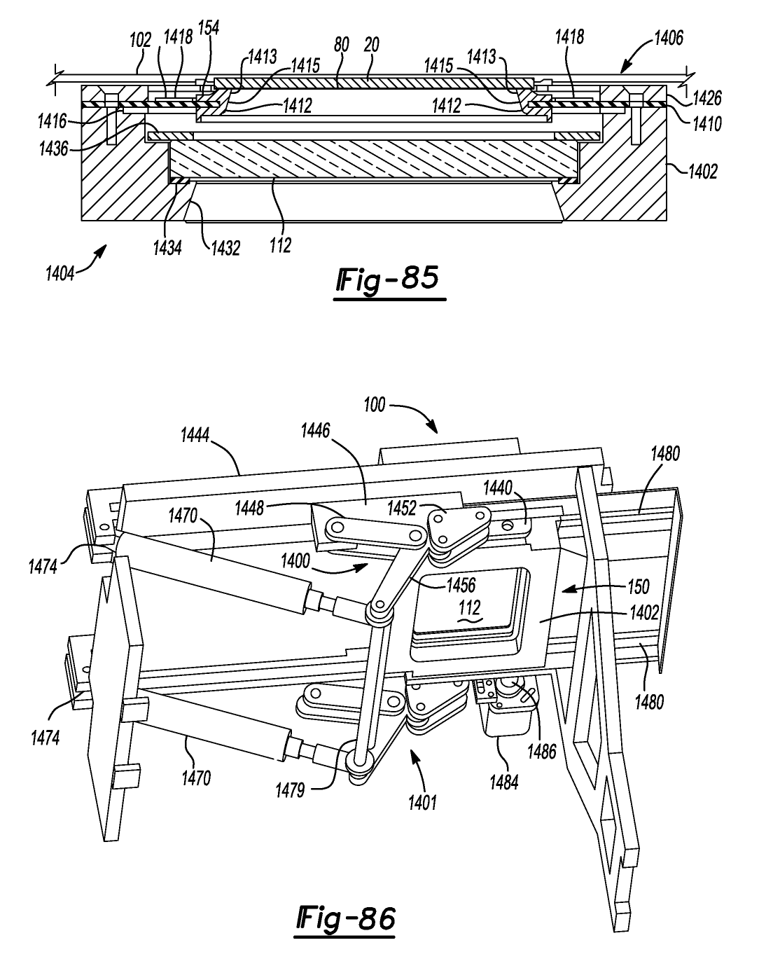

Turning now to FIGS. 84 and 85, one of some embodiments of pressure chamber 150 is illustrated. A chamber body 1402 has a first side 1404 and a second side 1406. In some embodiments, chamber body 1402 can be formed from aluminum or other materials such as steel, stainless steel, standard plastic, or fiber-reinforced plastic compound, such as a resin or polymer, and mixtures thereof. An opening 1408 extends through first side 1404 and second side 1406.

A chamber cover 1410 has an opening 1412 surrounded by circumferential chamber seal 154. Circumferential chamber seal 154 can have a peripheral lip that 1413 that defines a sealing plane abutting sealing cover 80 of microplate 20. In some embodiments, peripheral lip 1413 can be positioned radially inward of a periphery of opening 1412. A reactive surface 1415 can span between opening 1412 and peripheral lip 1413. Reactive surface 1415 can react to fluid pressure in pressure chamber 150 by increasingly urging peripheral lip 1413 against sealing cover 80 as the fluid pressure increases from zero to about 25 pounds per square inch (PSI). In some embodiments, chamber cover 1410 is formed from stainless steel. In some embodiments, a gasket 1414 (FIG. 85) can fit in a groove 1416 formed in a periphery of opening 1408 and provide a seal between chamber cover 1410 and chamber body 1402. Chamber cover 1410 can be as thin as practicable and have a lower thermal mass than said chamber body to reduce heat flow between microplate 20 and chamber body 1402. In some embodiments, frame 152 (also seen in FIG. 35) can comprise chamber cover 1410 and chamber body 1402.

In some embodiments, a thin film heater 1418 can be positioned on chamber cover 1410 to further reduce heat flow into chamber body 1402. Thin film heater 1418 can have a heater signal input 1420 to receive heater power from control system 1010. In some embodiments, a thermocouple 1422 can be positioned on chamber cover 1410 and provide a cover temperature signal 1424, by way of non-limiting example, via leads or other signal transmission medium, to control system 1010. Thermocouple 1422 can comprise, by way of non-limiting example, a type E, type J, type K, or type T thermocouple. Control system 1010 can use cover temperature signal 1424 to control heater power applied to thin film heater 1418 and thereby reduce temperature differences across microplate 20. In some embodiments, thin film heater 1418 can have a power dissipation of at least 50 watts.

In some embodiments, circumferential chamber seal 154 can be molded from a silicone material. In some embodiments, circumferential chamber seal 154 can be insert-molded with chamber cover 1410. An alignment ring 1426 can be fastened to chamber body 1402 through chamber cover 1410, and secure chamber cover 1410 to second side 1406. Microplate 20 can fit within an inner periphery of alignment ring 1426. Alignment ring 1426 can locate microplate 20 with respect to thermocycler system 100. In some embodiments, an alignment feature 1428 can interface with alignment feature 58 of microplate 20. In some embodiments, recesses 1430 can be formed in the inner periphery of alignment ring 1426. Recesses 1430 reduce a contact area between alignment ring 1426 and microplate 20 and can thereby reduce heat flow between microplate 20 and alignment ring 1426.

On first side 1404, a flange 1432 can protrude radially inward from the periphery of opening 1408 and support a window seal 1434. In some embodiments, flange 1432 can be about ¼″ wide. A surface of transparent window 112 can abut window seal 1434. In some embodiments, for example when window seal 1434 is a non-adhesive type seal, a window-retaining ring 1436 can be secured to chamber body 1402 and clamp transparent window 112 against window seal 1434. A connector 1438 can provide a connection to port 120 (FIGS. 34-37, 39-40) that is in fluid communication with the internal volume of pressure chamber 150.

At least one catch 1440 can be positioned on frame 152. In some embodiments, a pair of catches 1440 can be positioned on opposing sides of a perimeter of frame 152. Each of the pair of catches 1440 can have a centering feature 1442.

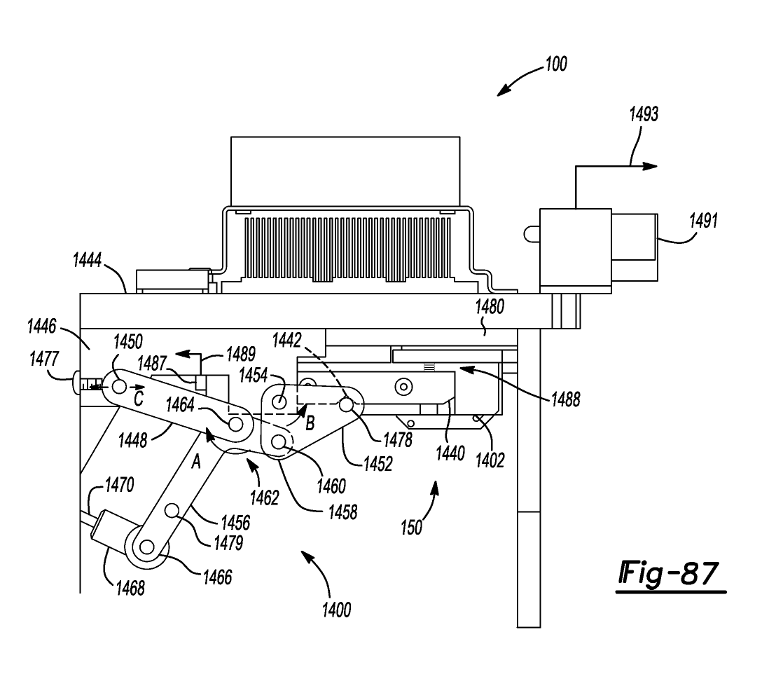

Referring now to FIGS. 86-88, thermocycler system 100 and clamp mechanism 1400 are illustrated fixedly mounted to a support structure 1444. In some embodiments, support structure 1444 can be generally planar in construction and adapted to be mounted within housing 1008 (FIG. 1). Clamp mechanism 1400 can be movable to between a locked condition (FIG. 86) and an unlocked condition (FIG. 87) and can be adapted to selectively clamp pressure chamber 150 against thermocycler system 100. An opening can be provided in support structure 1444 to allow contact between pressure chamber 150 and thermocycler system 100. In the locked condition, clamp mechanism 1400 can secure pressure chamber 150 in a clamped position against thermocycler system 100. In the clamped position, circumferential chamber seal 154 can be pressed against sealing cover 80 (best seen in FIG. 85). In the unlocked condition, clamp mechanism 1400 can allow pressure chamber 150 to be moved to an unclamped position away from thermocycler system 100. In some embodiments, the unclamped position can provide a gap of ⅜ inch between thermocycler block 102 (FIG. 85) and microplate 20. In some embodiments, clamp mechanism 1400 can be actuated manually. In other embodiments, clamp mechanism 1400 can be actuated by pneumatics, hydraulics, electric machines and/or motors, electromagnetics, or any other suitable means.

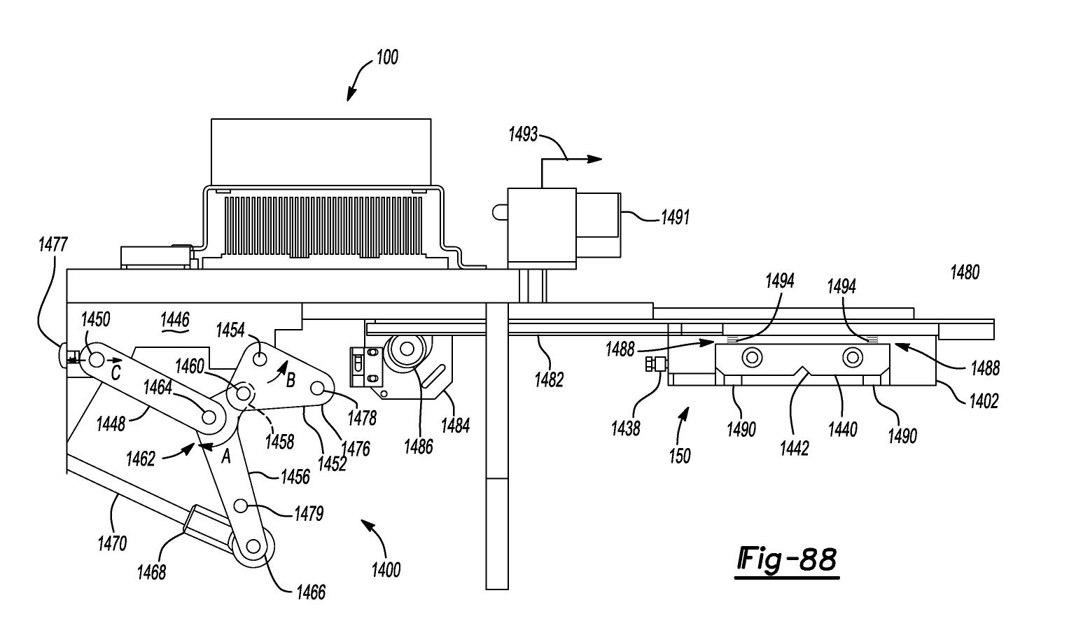

In some embodiments, clamp mechanism 1400 can have a clamp frame 1446 fixedly mounted to support structure 1444. An over-center link 1448 can pivot about a first end 1450 that can be pivotally connected to clamp frame 1446. A bellcrank 1452 can pivot about a pivot pin 1454 connected to clamp frame 1446. A lever arm 1456 can have a clamp end 1458 pivotally connected to an input end 1460 of bellcrank 1452. Lever arm 1456 can have an intermediate portion 1462 pivotally connected to a second end 1464 of over-center link 1448. An input end 1466 of lever arm 1456 can be pivotally connected to a telescoping end 1468 of a pneumatic cylinder 1470. A ball joint 1472 can pivotally connect telescoping end 1468 to input end 1466. A mounting end 1474 of pneumatic cylinder 1470 can pivotally connect to support structure 1444. In various other embodiments, mounting end 1474 of pneumatic cylinder 1470 can pivotally connect to clamp frame 1446. Bellcrank 1452 can have a clamp end 1476. A clamp pin 1478 can project from clamp end 1476 and engage centering feature 1442 when clamp mechanism 1400 is in the locked condition. It should be appreciated that the clamp mechanism 1400 on one side of thermocycler system 100 has been described. A second clamp mechanism 1401 can be positioned on the other side of thermocycler system 100 (FIG. 88). Second clamp mechanism 1401 can be symmetrical with the side just described and operate similarly. A transverse member 1479 can connect lever arm 1456 to the lever arm of the other side.

Operation of the clamp assembly 1400 embodiment illustrated in FIGS. 86-88 will now be described. Pneumatic cylinder 1470 can be movable between an extended condition (FIG. 87) and a contracted condition (FIGS. 86 and 88). As pneumatic cylinder 1470 moves to the contracted condition, it can cause lever arm 1456 to pivot as indicated by a curved arrow A. Lever arm 1456 can in turn cause bellcrank 1452 to pivot as indicated by a curved arrow B, thereby moving clamp pin 1478 towards centering feature 1442. Clamp pin 1478 can then become centered in centering feature 1442. As bellcrank 1452 completes rotating in the direction of arrow B, it can cause clamp pin 1478 to move chamber 150 from an unclamped position towards the clamped position against thermocycler assembly 100. This can cause circumferential chamber seal 154 to press against microplate 20 (best seen in FIG. 85). A clamping pressure between chamber seal 154 and microplate 20 can be adjusted by varying the pivot location of first end 1450 of over-center link 1448. In some embodiments, an adjustment mechanism 1477, such as, by way of non-limiting example, a screw, can be used to vary the pivot location as indicated by arrows A (FIG. 87).

Moving clamp mechanism 1400 to the unlocked condition will now be described. As pneumatic cylinder 1470 moves to the extended condition, it can cause lever arm 1456 to pivot in a direction opposite curved arrow A. Lever arm 1456 can in turn cause bellcrank 1452 to pivot in a direction opposite curved arrow B, thereby relieving the clamping pressure between clamp pin 1478 and catch 1440. Clamp pin 1478 can then disengage from centering feature 1442. As bellcrank 1452 completes rotating in the direction opposite curved arrow B, it can cause clamp pin 1478 to move away from catch 1440, allowing chamber 150, with microplate 20, to move to the unclamped position away from thermocycler system 100.

In some embodiments, a pair of rails 1480 can be used to traverse pressure chamber 150 between a thermocycler position adjacent thermocycler system 100 (FIG. 86) and a loading position away from thermocycler system 100 (FIG. 87). In some embodiments, the loading position can be external of housing 1008. In such embodiments, housing 1008 has an aperture that allows pressure chamber 150 and rails 1480 to pass therethrough. In some embodiments, a position sensor 1487 can be positioned on support structure 1440 and provide a position signal indicative of pressure chamber 150 being in the thermocycler position. In some embodiments, position sensor can be of an infrared, limit switch, contactless proximity, or ultrasonic type. Rails 1480 can be slidably mounted to support structure 1444. In some embodiments, optical sensor 1491 can read marking indicia 94 (FIG. 16) on microplate 20 as it is moved to the thermocycler position. Optical sensor 1491 can provide a marking data signal indicative of marking indicia 94 to control system 1010.

In some embodiments, rails 1480 can be telescoping rails. Rails 1480 can be moved manually or can be motorized. In some motorized embodiments, a rack gear 1482 can be positioned on at least one of rails 1480. A rotating actuator 1484 can be adapted with a pinion gear 1486 that engages rack gear 1482. Rotating actuator 1484 can rotate in response to control signals from control system 1010. In some embodiments, rotating actuator 1484 can be an electric motor, such as a stepper motor. For example, actuator 1484 can be a Vexta PK245-02AA stepper motor available from Oriental Motor U.S.A. Corp. In other embodiments, rotating actuator 1484 can be pneumatic or hydraulic. Pressure chamber 150 can be attached between rails 1480.

In some embodiments, a lost motion mechanism 1488 can be positioned between rails 1480 and pressure chamber 150. Lost motion mechanism 1488 can allow pressure chamber 150 limited perpendicular movement with respect to rails 1480. The limited perpendicular movement facilitates moving pressure chamber 150 between the clamped and unclamped positions as clamp assembly 1400 moves between the locked and unlocked conditions, respectively.

In some embodiments, lost motion mechanism 1488 can include shoulder bolts 1490 threaded into rails 1480. Catches 1440 can have through holes 1492 that slidingly engage shoulder bolts 1490. In some embodiments, springs 1494 can be positioned between catches 1440 and rails 1480. Springs 1494 can bias pressure chamber 140 toward the unclamped position and facilitate moving it away from thermocycler assembly 100 when clamp assembly 1400 moves to the unlocked condition.

### Microplate Clamping Adapters

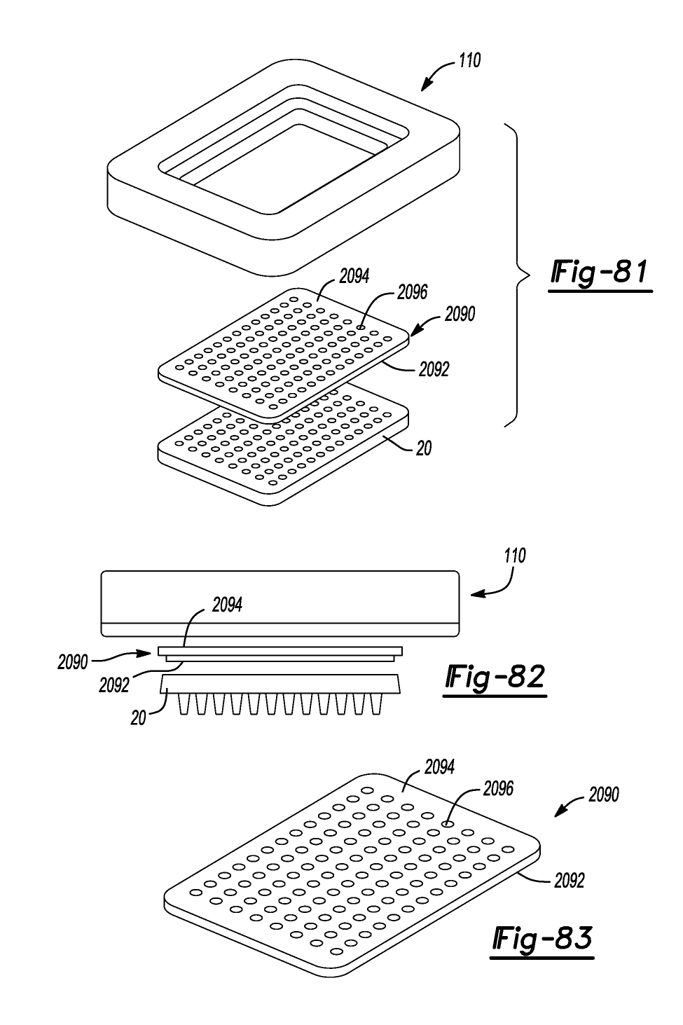

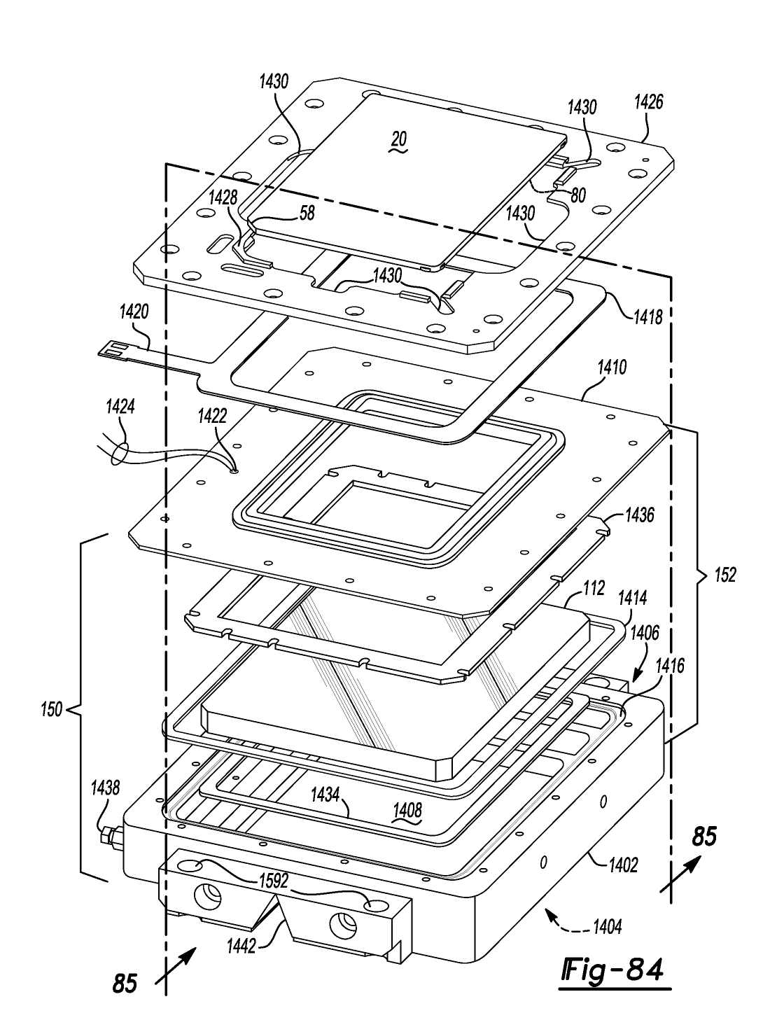

In some embodiments, it is useful to provide backwards compatibility of clamp mechanism 1400 (illustrated in FIGS. 86-88) with existing microplates or varying microplate shapes. This can pose a challenge when considering microplates having 96, 384, 1536, or more wells due to the decreasing amount of available surface area for engagement by any clamp mechanism. As illustrated in FIGS. 81-83, a clamp adapter 2090 can be used to accommodate these variations. In some embodiments, clamp adapter 2090 can be a structural member that includes a first side 2092 sized to receive or mate with the microplate and an opposing side 2094 sized to engage a clamping mechanism. In some embodiments, clamp adapter 2090 can comprise a plurality of through holes 2096 generally aligned with wells 26 of microplate 20 when clamp adapter 2090 is coupled therewith. In some embodiments, clamp adapter 2090 can be made from aluminum, steel, a stiff polymer, or the like.

Clamp adapter 2090 can translate the initial clamping motion of a clamp mechanism into a clamping force. In some embodiments, acting much like a mechanical clamp, clamp adapter 2090 can impart a clamping force on sealing cover 80 to assist in the sealing of wells 26 of microplate 20 undergoing thermocycling. In some embodiments, clamp adapter 2090 can be heated independently to control condensation on sealing cover 80 similar to the heated covers discussed herein. In some embodiments, depending on the cost of manufacture and the need for heating, clamp adapter 2090 can be a disposable consumable.

### Pneumatic System

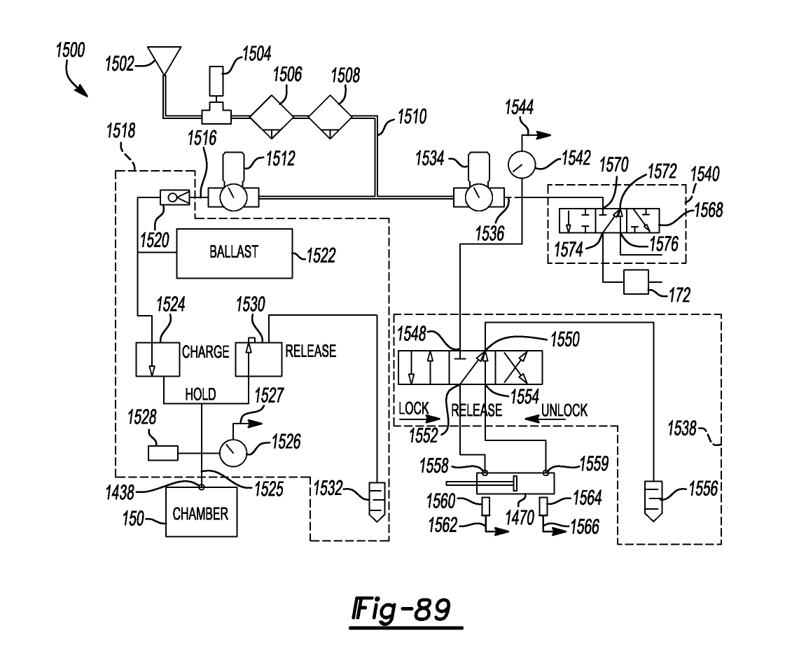

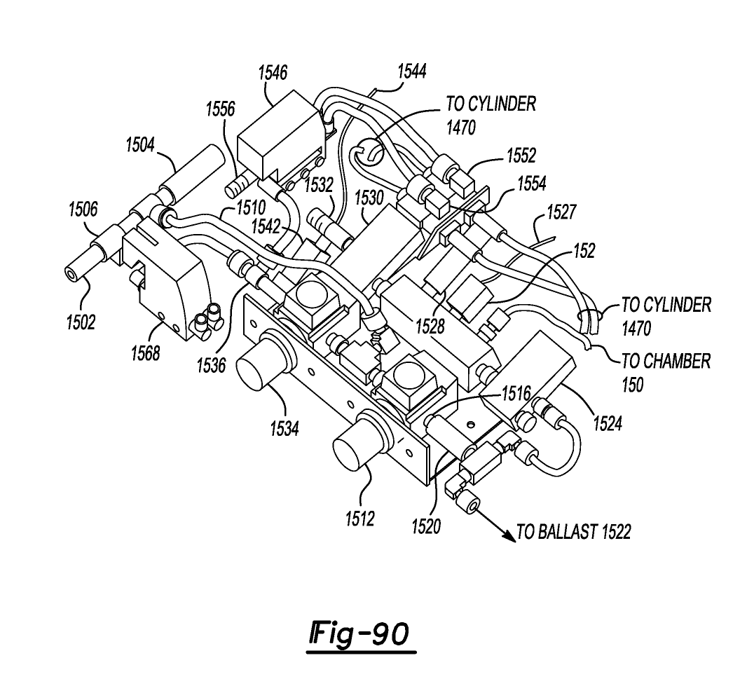

Referring now to FIGS. 89 and 90, a pneumatic system 1500 is illustrated in accordance with some embodiments. Pneumatic system 1500 can provide pneumatic control for various pneumatic devices used in sequence detection system 10. By way of non-limiting example, the pneumatic devices can include, alone or in any combination, pressure chamber 150, pneumatic cylinders 1470, and vacuum source 172.

An input coupling 1502 can provide a connection point for a supply of compressed fluid, such as, by way of non-limiting example, air, but can also comprise nitrogen, argon, or helium. Input coupling 1502 can be accessible from an exterior of housing 1008 (FIG. 1). In some embodiments, a pressure relief valve 1504 can be in fluid communication with input coupling 1502. In some embodiments, pressure relief valve 1504 can have a maximum pressure of 120 PSI. In some embodiments, a particle filter 1506 can be in fluid communication with pressure relief valve 1504. In some embodiments, a condensation separator 1508 can be in fluid communication with particle filter 1508. Alternatively, condensation separator 1508 can be in fluid communication with pressure relief valve 1504. Particle filter 1506 and condensation separator 1508 can provide a conditioned fluid supply 1510 to a remainder of pneumatic system 1500.

In some embodiments, a first pressure regulator 1512 can be in fluid communication with conditioned fluid supply 1510. First pressure regulator 1512 can provide a first fluid supply 1516 to a chamber pressurization subsystem 1518 and/or to other subsystems.

In chamber pressurization subsystem 1518, a check valve 1520 can be connected in series with first pressure regulator 1512. Check valve 1520 can reduce a risk of depressurization of the internal volume of pressure chamber 150 in the event conditioned fluid supply 1510 is interrupted. A ballast tank 1522 can be in fluid communication with the first fluid supply 1516 and increase a fluid volume of chamber pressurization subsystem 1518. The increased volume can reduce pressure variations of the first fluid supply 1516. Ballast tank 1522 can also provide a fluid reserve to help maintain pressure in the event first fluid supply 1516 is interrupted. One side of a charge valve 1524 can be in fluid communication with the first fluid supply 1516. The other side of charge valve 1524 can be in fluid communication with the internal volume of pressure chamber 150. A flexible fluid line can connect chamber pressurization subsystem 1518 to connector 1438 of chamber 150. Charge valve 1524 can be controlled by control system 1010 in accordance with a method described later herein. In some embodiments, charge valve 1524 can be a part number MKH0NBG49A available from Parker-Hannifin Corp.

A pressure sensor 1526 can be in fluid communication with the internal volume of pressure chamber 150 and can provide a chamber pressure signal 1527 to control system 1010. In some embodiments, pressure sensor 1526 can be a part number MPS-P6N-AG available from Parker-Hannifin Corp. A chamber pressure relief valve 1528 can be in fluid communication with the internal volume of pressure chamber 150 and establish a maximum pressure that can be applied thereto. In some embodiments, the maximum pressure of 1528 chamber pressure relief valve can be less than, or equal to, 30 PSI.

Pressurization subsystem 1518 can also comprise a release valve 1530 in fluid communication with the internal volume of pressure chamber 150. The other side of release valve 1530 can be vented to atmosphere. Release valve 1530 can be controlled by control system 1010 in accordance with a method described later herein. In some embodiments, release valve 1530 can be a part number MKH0NBG49A available from Parker-Hannifin Corp. In some embodiments, the charge and release valves 1524, 1530 can maintain chamber pressure at about 18 PSI while the microplate temperature is greater than 40 degrees Celsius. This combination of pressure and temperature conditions can help reduce a possibility of pressure within wells 26 overcoming the chamber pressure and causing wells 26 to leak between sealing cover 80. A first silencer 1532 can be in fluid communication with the other side of release valve 1530 to reduce noise as fluid is vented.

In some embodiments, a second pressure regulator 1534 can be in fluid communication with conditioned fluid supply 1510. Second pressure regulator 1534 can provide a second fluid supply 1536 to a cylinder control subsystem 1538. Second pressure regulator 1540 can also provide second fluid supply 1536 to a vacuum control subsystem 1540. A pressure transducer 1542 can be in fluid communication with second fluid supply 1536 and provide a pressure signal 1544 to control system 1010. In some embodiments, pressure transducer 1542 can comprise a part number MPS-P6N-AG available from Parker-Hannifin Corp. In some embodiments, second fluid supply 1536 is greater than, or equal to, 50 PSI.

In cylinder control subsystem 1538, a cylinder valve 1546 can have a pressure port 1548, an exhaust port 1550, a first port 1552, and a second port 1554. Cylinder valve 1546 can be referred to as a 3-position, 2-port valve, commonly referred to as a 3/2 valve. In some embodiments, cylinder valve 1546 can comprise a part number P2MISGEE2CV2DF7 available from Parker-Hannifin Corp. or a part number B360(c)A549C available from Parker-Hannifin Corp. Pressure port 1548 can be in fluid communication with second fluid supply 1536. Exhaust port 1550 can be vented to atmosphere. Cylinder silencer 1556 can be in fluid communication with exhaust port 1550 to reduce noise when fluid is vented from pneumatic cylinder 1470. First port 1552 can be in fluid communication with first port 1558 of pneumatic cylinder 1470. Second port 1554 can be in fluid communication with second port 1559 of pneumatic cylinder 1470. Cylinder valve 1546 can be manually controlled. In some embodiments, cylinder valve 1546 is a servovalve controlled by control system 1010 in accordance with a method described later herein.

Cylinder valve 1546 can have three positions that route fluid between ports 1548-1554. A first position can route pressure port 1548 to first port 1552 and route second port 1554 to exhaust port 1550. A second position can block pressure port 1548 and route first and second ports 1552, 1554 to exhaust port 1550. A third position can route pressure port 1548 to second port 1554 and route first port 1552 to exhaust port 1550. The first, second, and third positions of cylinder valve 1546 can be referred to as the lock, release, and unlock positions, respectively.

When cylinder valve 1546 is in the lock position, fluid routing through cylinder valve 1546 can cause pneumatic cylinder 1470 to move to the contracted condition, thereby moving clamp mechanism 1400 to the locked condition (FIG. 86). When cylinder valve 1546 is in the unlock position, the fluid routing through cylinder valve 1546 can cause pneumatic cylinder 1470 to move to the extended condition, thereby moving clamp mechanism 1400 to the unlocked condition (FIG. 87). When cylinder valve 1546 is in the release position, the fluid routing through cylinder valve 1546 can cause pneumatic cylinder 1470 to be freely extended or contracted by an outside influence, thereby allowing clamp mechanism 1400 to be manually moved between the closed and open positions. It should be noted that over-center link 1448 can maintain clamp mechanism in the locked condition when cylinder valve 1546 is moved to the release position. A first limit switch 1560 can sense, either directly or indirectly, when pneumatic cylinder 1470 is in the extended condition and provide a corresponding signal 1562 to control system 1010. A second limit switch 1564 can be used to sense, either directly or indirectly, when pneumatic cylinder 1470 is in the contracted condition and provide a corresponding signal 1566 to control system 1010. In some embodiments, first and second limits switches 1560, 1564 can be integral to pneumatic cylinder 1470. In some embodiments, pneumatic cylinder 1470 can be a Parker-Hannifin Corp. SRM Series pneumatic cylinder with piston sensing capability. In some embodiments, pneumatic cylinder 1470 can be a part number L06DP-SRMBSY400 from Parker-Hannifin Corp.

In some embodiments, vacuum control system 1540 selectively actuates vacuum source 172. Vacuum generated by vacuum source 172 can be provided to thermocycler system 100 or other systems. Vacuum control system 1572 can comprise a vacuum control valve 1568. In some embodiments, vacuum control valve 1568 can comprise a part number P2MISDEE2CV2BF7 available from Parker-Hannifin Corp.

Vacuum control valve 1568 can have a pressure port 1570, an exhaust port 1572, a first port 1574, and a second port 1576. Vacuum control valve 1568 can be referred to as a 3-position, 2-port valve, commonly referred to as a 3/2 valve. Pressure port 1570 can be in fluid communication with second fluid supply 1536. In some embodiments, exhaust port 1572 can be blocked. In other embodiments, exhaust port 1572 can be vented to atmosphere. First port 1574 can be in fluid communication with vacuum source 172. Second port 1576 can be blocked in some embodiments having exhaust port 1572 vented to atmosphere. In other embodiments, second port 1576 can be vented to atmosphere. Vacuum control valve 1568 can be manually controlled. In some embodiments, vacuum control valve 1568 is a servovalve controlled by control system 1010 in accordance with a method described later herein.

Vacuum control valve 1568 can have three positions that route fluid between ports 1570-1576. A first position can route pressure port 1570 to first port 1574, and can block exhaust port 1572 and second port 1576. A second position can block pressure port 1570, and route first and second ports 1574, 1576 through exhaust port 1572. A third position can route pressure port 1570 to second port 1576, and block first port 1574 and exhaust port 1572. The first, second, and third positions of vacuum control valve 1568 can also be referred to as the vacuum on, vacuum off, and vent positions, respectively.

When vacuum control valve 1568 is in the vacuum on position, the fluid routing through vacuum control valve 1568 can flow through vacuum source 172. Vacuum source 172 generates a vacuum in response thereto that can be fluidly coupled to the thermocycler system 100 or other systems. When vacuum control valve 1568 is in the vacuum off position, second fluid supply 1536 is disconnected from vacuum source 172 and vacuum source 172 can be routed to atmosphere through exhaust port 1572 and/or second port 1576. When vacuum control valve 1568 is in the vent position, second fluid supply 1536 can be purged to atmosphere through second port 1576.

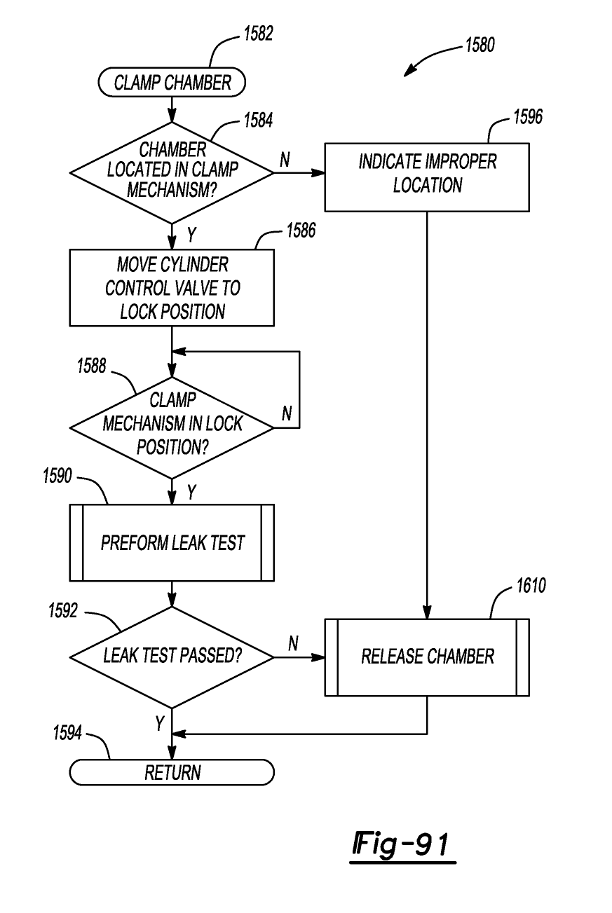

Referring now to FIG. 91, a method 1580 is illustrated, according to some embodiments, for clamping pressure chamber 150 to thermocycler system 100. Method 1580 can be executed by control system 1010 when pressure chamber 150 is placed in proximity to thermocycler block 102. Method 1580 can begin in step 1582 and can proceed to decision step 1584 to determine whether pressure chamber 150 is properly located within clamp mechanism 1400. Position signal 1489 (FIG. 86) can be used to make the determination. When pressure chamber 150 is properly located, method 1580 can proceed to step 1586 and move cylinder valve 1546 to the lock position. Method 1580 can then proceed to decision step 1588 and determine whether pneumatic cylinder 1470 has moved to the contracted condition, thereby placing clamp mechanism 1400 in the locked condition. Decision step 1588 can make the determination by using signal 1566 (FIG. 89) from second limit switch 1570. Method 1580 can execute decision step 1588 until pneumatic cylinder 1470 moves to the contracted condition. Method 1580 can then proceed to step 1590 and can perform a leak test 1590 as described later herein. Method 1580 can then proceed to decision step 1592 and determine, from results of leak test 1590, whether leak test 1590 passed. If leak test 1590 passed, then method 1580 can proceed to step 1594 and exit. If leak test 1590 failed, then method 1580 can proceed to step 1610 and release chamber 150 according to a method described later herein.

Returning to decision step 1584, if method 1580 determines that chamber 150 is improperly located within clamp mechanism 1400, then method 1580 can proceed to step 1596. In step 1596, method 1580 can indicate that chamber 150 is improperly located within clamp mechanism 1400. Method 1580 can then proceed to method 1610 and assure clamp mechanism 1400 is in the unlocked condition. Method 1580 can indicate the improper location of chamber 150 though, by way of example, a buzzer, lamp, writing to a computer memory in control system 1010, or any other suitable means.

Referring now to FIG. 92, method 1590 is illustrated, according to some embodiments of the invention, for performing the leak test on chamber 150. Method 1590 can be executed by control system 1010 when chamber 150 is in the clamped position. Method 1590 can begin at step 1591 and can proceed to step 1593. In step 1593, method 1590 can pressurize chamber 150 by opening charge valve 1524 and closing release valve 1530 (FIG. 89). Method 1590 can then proceed to decision step 1595 and determine a chamber leak rate of pressure chamber 150. In one of some embodiments, the chamber leak rate can be determined by determining a difference in air pressure, as indicated by pressure transducer 1526, over a predetermined amount of time. In one example, the chamber leak rate can be expressed in units of PSI/minute. In decision step 1595, method 1590 can compare the chamber leak rate to a predetermined leak rate. If the chamber leak rate is less than the predetermined leak rate, method 1590 can proceed to step 1598, indicating that the leak test has passed. Method 1590 can then proceed to step 1600 and open charge valve 1524 to connect ballast tank 1536 to the internal volume of pressure chamber 150. In step 1600, method 1590 can also provide an indication to control system 1010 that thermocycling can begin.

Returning now to decision step 1595, if the chamber leak rate is greater than, or equal to, the predetermined leak rate, method 1590 can proceed to step 1602, indicating that the leak test has failed. Method 1590 can then proceed to step 1604 and indicate the failure though, by way of example, a buzzer, lamp, writing to the computer memory in control system 1010, or any other suitable means. Method 1590 can exit at step 1606 from either step 1600 or step 1604.

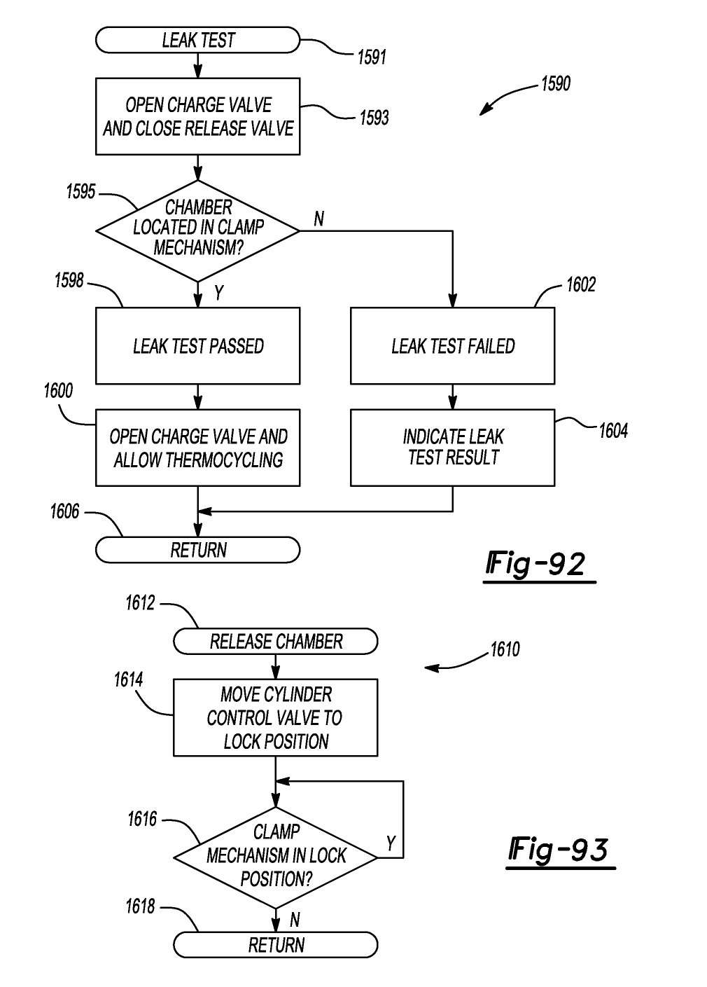

Referring now to FIG. 93, method 1610 of unclamping pressure chamber 150 from thermocycler system 100 is illustrated according to one of several embodiments. Method 1610 can be executed by control system 1010. In some embodiments, method 1612 can be called by method 1580. Method 1610 can also be executed after thermocycling is completed. Method 1610 can begin in step 1612 and then can proceed to step 1614. In step 1614, method 1610 can move cylinder valve 1546 to the unlock position, which can cause pneumatic cylinder 1470 to begin moving to the extended condition and changing clamp mechanism to the unlocked condition. Method 1610 can then proceed to decision step 1616 and determine whether pneumatic cylinder 1470 has moved to the extended condition. Decision step 1616 can make the determination by using signal 1562 (FIG. 89) from first limit switch 1560. Method 1610 can execute decision step 1616 until pneumatic cylinder 1470 moves to the extended condition. Method 1610 can then proceed to step 1618 and exit.

### Excitation System

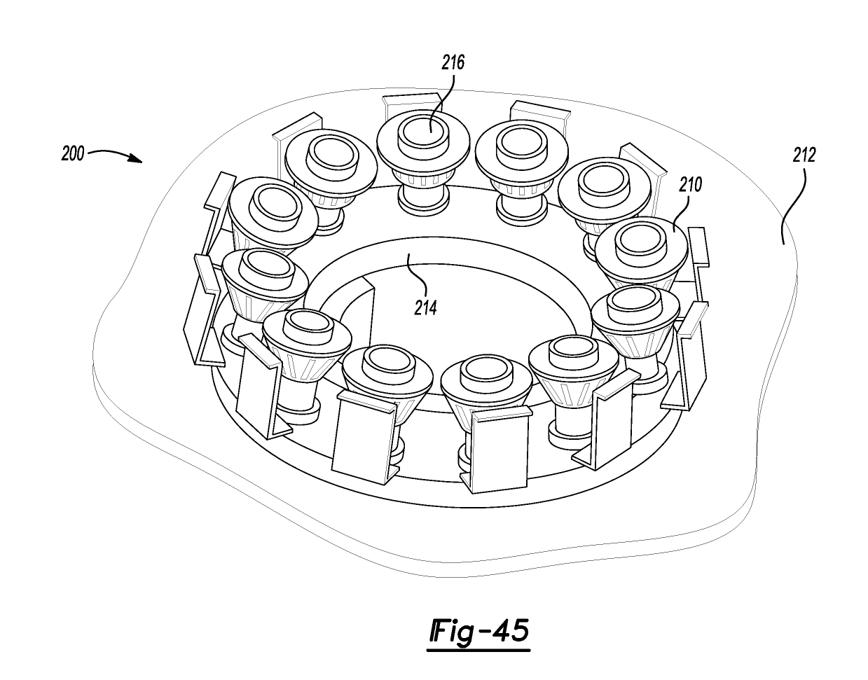

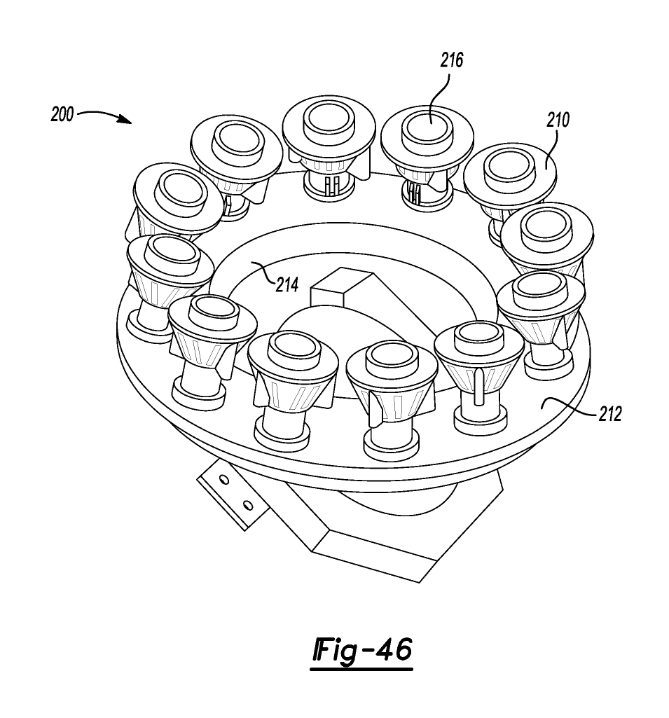

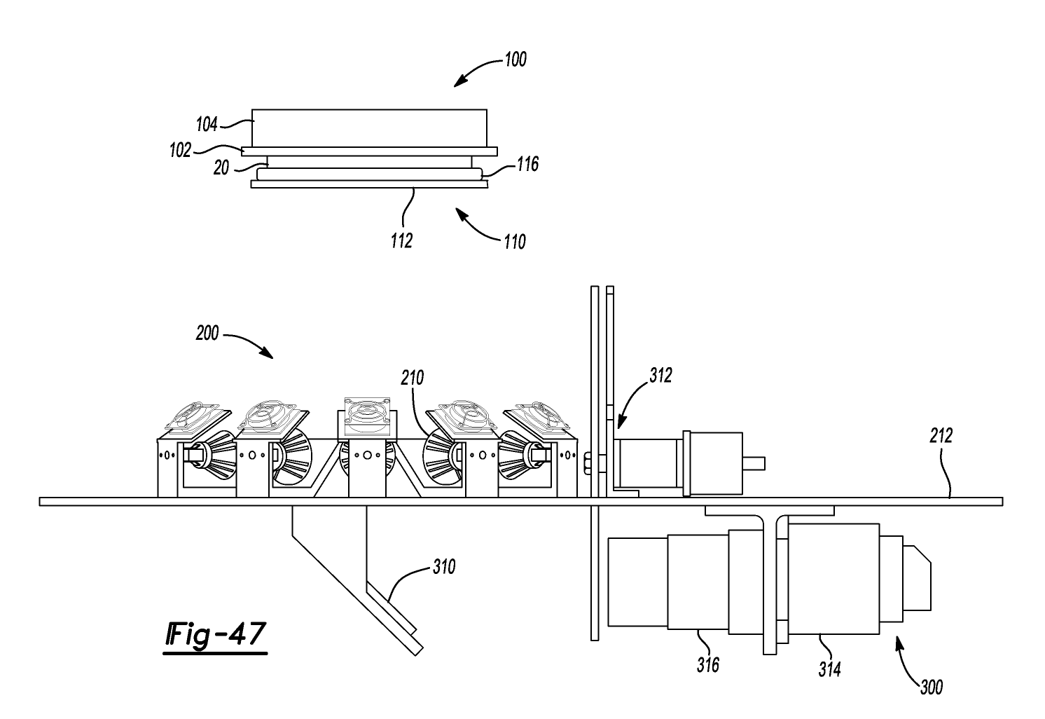

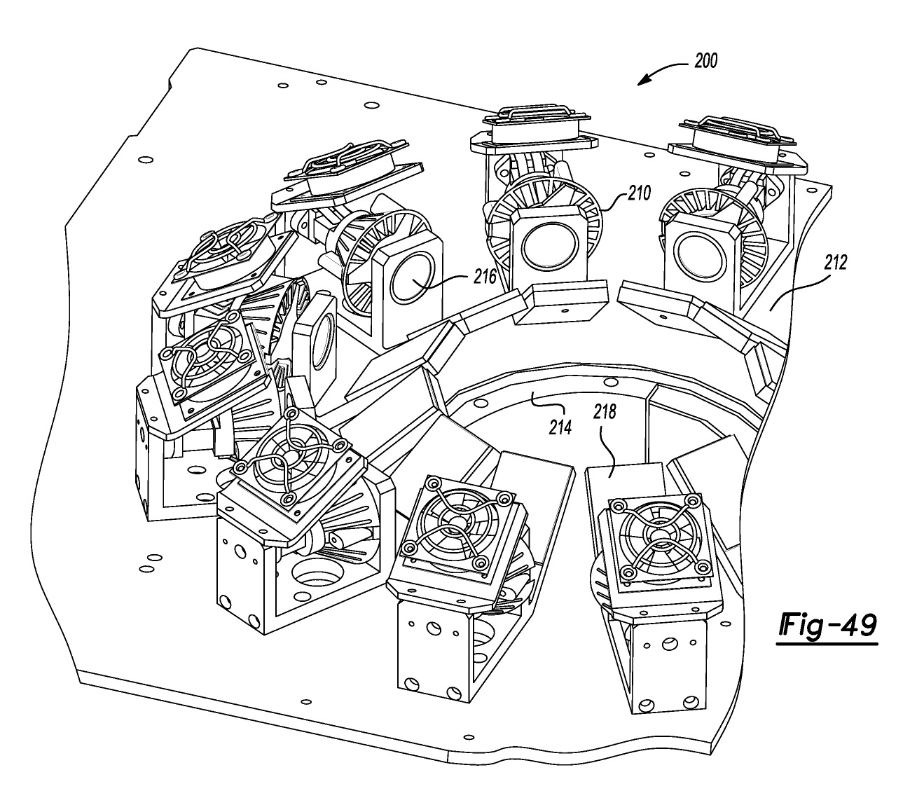

In some embodiments, as illustrated in FIGS. 42-49, excitation system 200 generally comprises a plurality of excitation lamps 210 generating excitation light 202 in response to control signals from control system 1010. Excitation system 200 can direct excitation light 202 to each of the plurality of wells 26 or across the plurality of wells 26. In some embodiments, excitation light 202 can be a radiant energy comprising a wavelength that permits detection of photo-emitting detection probes in assay 1000 disposed in at least some of the plurality of wells 26 of microplate 20 by detection system 300.

By way of background, it should be understood that the quantitative analysis of assay 1000, in some embodiments, can involve measurement of the resultant fluorescence intensity or other emission intensity. In some embodiments of the present teachings, fluorescence from the plurality of wells 26 on microplate 20 can be measured simultaneously using a CCD camera. In an idealized optical system, if all of the plurality of wells 26 have the same concentration of dye, each of the plurality of wells 26 would produce an identical fluorescence signal. In some prior conventional designs, wells near the center of the microplate may appear significantly brighter (i.e. output more signal) than those wells near the edge of the microplate, despite the fact that all of the wells may be outputting the same amount of fluorescence. There are several reasons for this condition in some current designs-vignetting, shadowing, and the particular illumination/irradiance profile.

With respect to vignetting, camera lenses can collect more light from the center of the frame relative to the edges. This can reduce the efficiency of certain prior, conventional detection systems. Additionally, in certain prior, conventional designs, the irradiance profile is sometimes not uniform. Most commercially available irradiance sources have a greater irradiance value (watts/meter²) near the center compared to the edges of the irradiance zone. In PCR, it has been found that for a given dye, until the dye saturates or bleaches, the amount of fluorescence can be proportional to the irradiance of the illumination source. Therefore, if the excitation light is brighter at the center, then the fluorescence signal from a well near the edge of the irradiance zone would be less than an identical well near the center. Shadowing can occur due to the depth of the wells. Unless the excitation light is perpendicular to the microplate, some part of the well may not be properly illuminated. In other words, the geometry of the well may block some of the light from reaching the bottom of the well. In addition, the amount of fluorescence emitted, which can be collected, may vary from center to edge. As should be appreciated by one skilled in the art, noise sources are often constant across the field of view of the camera. Therefore, for wells near the edges of microplate 20 that output a smaller amount of fluorescence, the signal to noise ratio can be adversely effected, thereby reducing the efficiency of high-density sequence detection system 10. As illustrated in FIG. 50, a graph illustrates the relative intensity or light transmission versus well location on a plate. As can be seen from the graph, the effects of vignetting and shadowing causes the light intensity to drop off along the edges of the field of view of the plate.

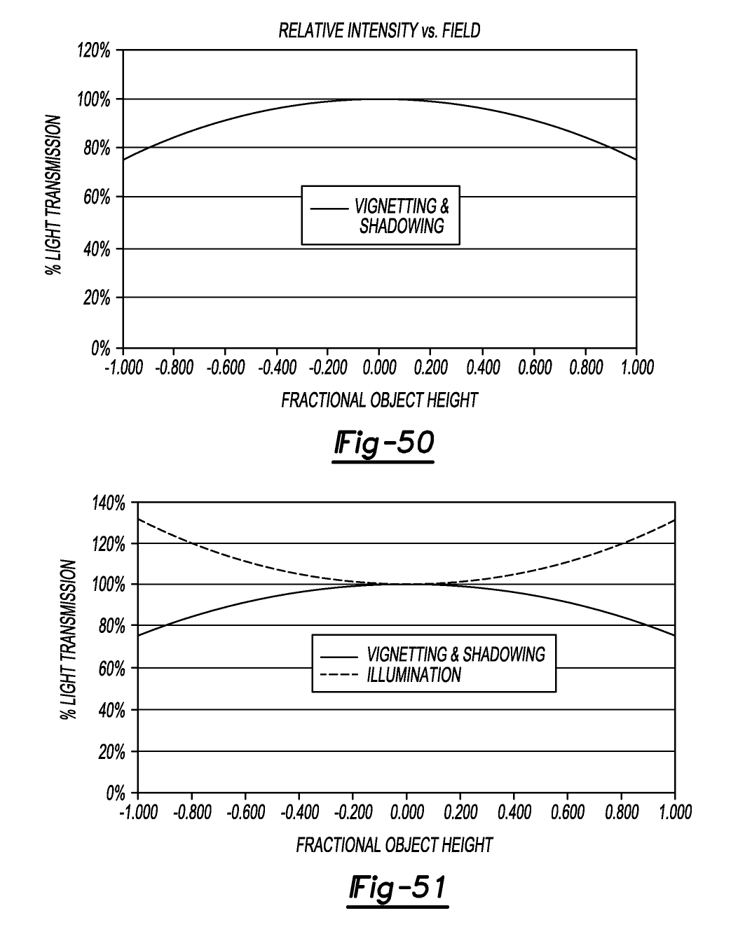

The present teachings, at least in part, address these effects so that identical wells output generally identical fluorescence irrespective of their location on microplate 20. By using the profile from FIG. 50, the optimum irradiance profile can be calculated. With reference to FIG. 51, a corresponding irradiance profile, represented by a dashed line, can provide a higher irradiance along the edges. This irradiance profile, when coupled with the effects of vignetting and shadowing, creates generally uniform signal strength across all of the plurality of wells 26 of microplate 20.

### Excitation Sources

In some embodiments, as illustrated in FIGS. 42-49, the plurality of excitation lamps 210 of excitation system 200 can be fixedly mounted to a support structure 212. In some embodiments, the plurality of excitation lamps 210 can be removably mounted to support structure 212 to permit convenient interchange, exchange, replacement, substitution, or the like. In some embodiments, support structure 212 can be generally planar in construction and can be adapted to be mounted within housing 1008 (FIG. 1). The plurality of excitation lamps 210 can be arranged in a generally circular configuration and directed toward microplate 20 to promote uniform excitation of assay 1000 in each of the plurality of wells 26. The present teachings permit a generally uniform excitation that is substantially free of shadowing. In some embodiments, the plurality of excitation lamps 210 can be arranged in a generally circular configuration about an aperture 214 formed in support structure 212. Aperture 214 permits the free transmission of fluorescence therethrough for detection by detection system 300, as described herein.

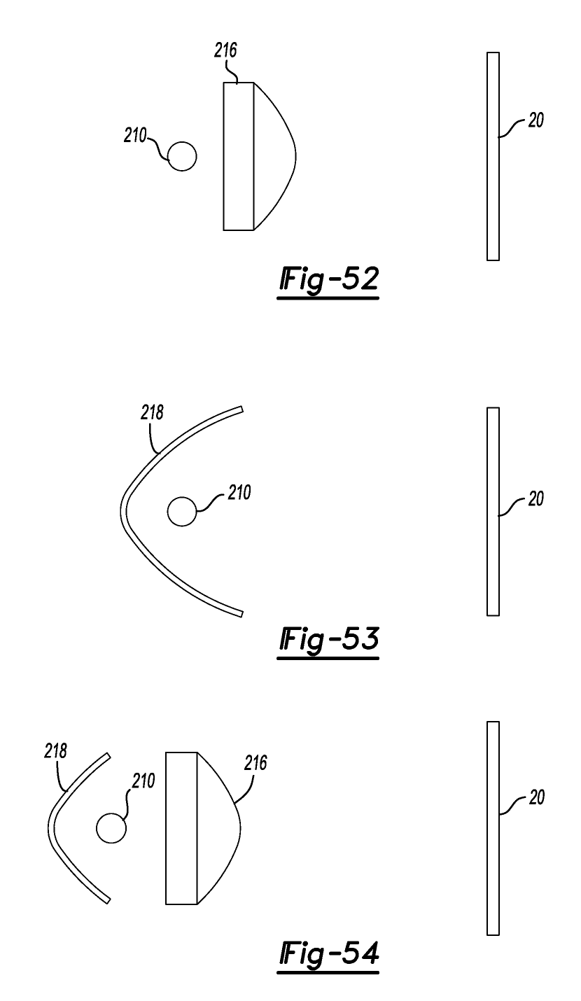

In some embodiments, as illustrated in FIGS. 52-56, each of the plurality of excitation lamps 210 can be configured to achieve the desired irradiance profile. In some embodiments, as seen schematically in FIG. 52, each of the plurality of excitation lamps 210 can comprise a lens 216. Lens 216 can be shaped to provide a desired irradiance profile (see FIG. 51). The exact shape of lens 216 can depend, at least in part, upon one or more of the desired irradiance profile at microplate 20, the illumination/irradiance profile at each of the plurality of excitation lamps 210, and the size and position of microplate 20 relative to the plurality of excitation lamps 210. The shape of lens 216 can be calculated in response to the particular application using commercially available software, such as ZEMAX and/or ASAP.

In some embodiments, as seen schematically in FIG. 53, each of the plurality of excitation lamps 210 can comprise a mirror 218. Mirror 218 can be shaped to provide a desired irradiance profile (see FIG. 51). The exact shape of mirror 218 can be dependent, at least in part, upon the desired irradiance profile at microplate 20, the illumination/irradiance profile at each of the plurality of excitation lamps 210, and the size and position of microplate 20 relative to the plurality of excitation lamps 210. The shape of mirror 218 can be calculated in response to the particular application using commercially available software, such as ZEMAX and/or ASAP.

In some embodiments, as illustrated in FIG. 54, each of the plurality of excitation lamps 210 can comprise a combination of lens 216 and mirror 218 to achieve the desired irradiance profile. Again, lens 216 and mirror 218 can be calculated in response to the particular application using commercially available software, such as ZEMAX and/or ASAP.

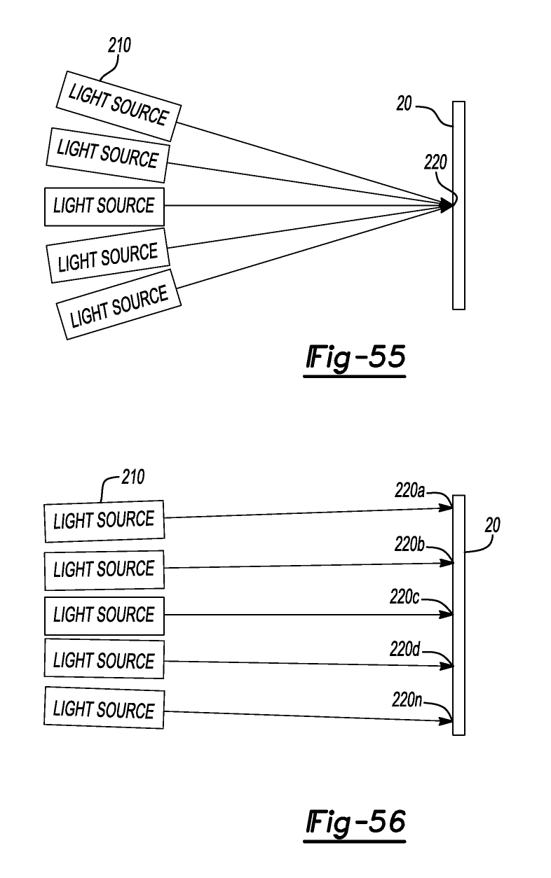

Turning now to FIG. 55, in some embodiments, each of the plurality of excitation lamps 210 can be aligned such that their optical centers converge on a single point 220. Additionally, in some embodiments, a desired irradiance profile (see FIG. 51) can be achieved by directing each of the plurality of excitation lamps 210 at a predetermined location 222a-222n on microplate 20, as illustrated in FIG. 56. In some embodiments, each of the plurality of excitation lamps 210 can comprise lens 216 and/or mirror 218 and can further be aligned as illustrated in FIG. 56 to achieve more complex irradiance profiles. As can be appreciated, employing any of the above techniques described herein can provide improved irradiance across microplate 20, thereby improving the resultant signal to noise ratio of the plurality of wells 26 along the edge of microplate 20.

It is anticipated that the plurality of excitation lamps 210 can be any one of a number of sources. In some embodiments, the plurality of excitation lamps 210 can be a laser source having a wavelength of about 488 nm, an Argon ion laser, an LED, a halogen bulb, or any other known source. In some embodiments, the LED can be a MR16 from Opto Technologies (Wheeling Ill.; http://www.optotech.com/MR16.htm). In some embodiments, the LED can be provided by LumiLEDS. In some embodiments, the halogen bulb can be a 75 W, 21 V DC lamp or a 50 W, 12 V DC lamp.

As discussed above, each of the plurality of excitation sources 210 can be removably coupled to support structure 212 to permit convenient interchange, exchange, replacement, substitution, or the like thereof. In some embodiments, the particular excitation source(s) employed can be selected by one skilled in the art to exhibit desired characteristics, such as increased power, better efficiency, improved uniformity, multi-colors, or having any other desired performance criteria. In embodiments employing multi-color and/or multi-wavelength excitation sources, additional detection probes and/or dyes can be used to, in some circumstances, increase throughput of high-density sequence detection system 10 by including multiple assays in each of the plurality of wells 26.

In some embodiments, the temperature of the plurality of excitation lamps 210 can be controlled to decrease the likelihood of intensity and spectral shifts. In such embodiments, the temperature control can be, for example, a cooling device. In some embodiments, the temperature control can maintain each of the plurality of excitation lamps 210 at an essentially constant temperature. In some embodiments, the intensity can be controlled via a photodiode feedback system, utilizing pulse width modulation (PWM) control to modulate the power of the plurality of excitation lamps 210. In some embodiments, the PWM can be digital. In some embodiments, shutters can be used to control each of the plurality of excitation lamps 210. It should be appreciated that any of the excitation assemblies 200 illustrated in FIGS. 42-49 and described above can be interchanged with each other.

### Detection Systems

In some embodiments, as illustrated in FIGS. 42-44, 47, and 48, detection system 300 can be used to detect and/or gather fluorescence emitted from assay 1000 during analysis. In some embodiments, detection system 300 can comprise a collection mirror 310, a filter assembly 312, and a collection camera 314. After excitation light 202 passes into each of the plurality of wells 26 of microplate 20, assay 1000 in each of the plurality of wells 26 can be illuminated, thereby exciting a detection probe disposed therein and generating an emission (i.e. fluorescence) that can be detected by detection system 300.

In some embodiments, collection mirror 310 can collect the emission and/or direct the emission from each of the plurality of wells 26 towards collection camera 314. In some embodiments, collection mirror 310 can be a 120 mm-diameter mirror having ¼ or 1/2 wave flatness and 40/20 scratch dig surface. In some embodiments, filter assembly 312 comprises a plurality of filters 318. During analysis, microplate 20 can be scanned numerous times—each time with a different filter 318.

In some embodiments, collection camera 314 comprises a multi-element photo detector 324, such as, but not limited to, charge coupled devices (CCDs), diode arrays, photomultiplier tube arrays, charge injection devices (CIDs), CMOS detectors, and avalanche photodiodes. In some embodiments, the emission from each of the plurality of wells 26 can be focused on collection camera 314 by a lens 316. In some embodiments, collection camera 314 is an ORCA-ER cooled CCD type available from Hamamatsu Photonics. In some embodiments, lens 316 can have a focal length of 50 mm and an aperture of 2.0. In some embodiments, collection camera 314 can be mounted to, and prealigned with, lens 316.

In some embodiments, detection system 300 can comprise a light separating element, such as a light dispersing element. Light dispersing element can comprise elements that separate light into its spectral components, such as transmission gratings, reflective gratings, prisms, beam splitters, dichroic filters, and combinations thereof that are can be used to analyze a single bandpass wavelength without spectrally dispersing the incoming light. In some embodiments, with a single bandpass wavelength light dispersing element, a detection system can be limited to analyzing a single bandpass wavelength. Therefore, one or more light detectors, each comprising a single bandpass wavelength light dispersing element, can be provided.

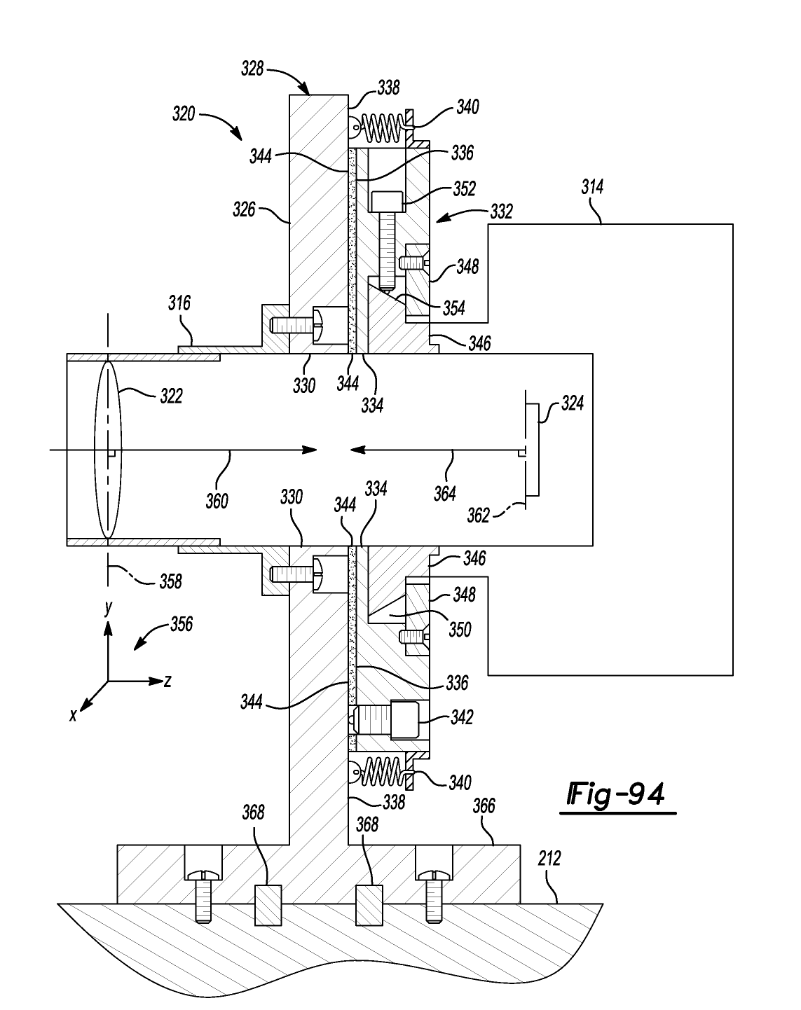

In some embodiments, as seen in FIG. 94, an alignment mount 320 can mate collection camera 314 and lens 316. Alignment mount 320 can provide a mechanism to adjust an axial alignment and a distance between an optic assembly 322 and multi-element photo detector 324. Lens 316 can receive optic assembly 322 and can mount to a mounting face 326 of a base plate 328. Base plate 328 can have an aperture 330 formed therein that can allow light to pass from optic assembly 322 to multi-element photo detector 324. In some embodiments, base plate 328 can be formed from a metal, such as steel, stainless steel, or aluminum.

Collection camera 314 can contain multi-element photo detector 324 and can mount to a camera mounting plate 332. Mounting plate 332 can have an aperture 334 that can align with aperture 330. Mounting plate 332 can have a face 336 generally parallel to a mating face 338 of base plate 328. In some embodiments, mounting plate 332 can be formed from a metal, such as steel, stainless steel, or aluminum. At least one resilient member 340 can attach to mounting plate 332 and to base plate 328. Resilient member 340 can be formed, by non-limiting example, from a spring and/or other elastic structure. Resilient member 340 can provide a bias force that urges face 336 towards mating face 338. A planarity adjustment feature, such as, by way of non-limiting example, at least one setscrew 342, can be positioned between face 336 and mating face 338. At least one setscrew 342 can apply a force opposite the bias force provided by resilient member 340 and maintain face 336 in a spaced relationship from mating face 338.

In some embodiments, at least one set screw 342 can have a thread pitch between 80 and 100 threads per inch (TPI), inclusive. In some embodiments, at least one setscrew 342 can be a ball-end type. In some embodiments, three setscrews 342 can be radially spaced around mounting plate 332. In some embodiments, the planarity adjustment feature can comprise cams, motorized screws, fluid-containing bags, or inclined planes. In some embodiments, the space between face 336 and mating face 338 can be less than ⅛ inch. In some embodiments, a light blocking gasket 344 can be positioned in the space between face 336 and mating face 338. In some embodiments, light blocking gasket 344 can be formed from closed cell foam. Light blocking gasket 344 can have apertures formed therein that align with apertures 330 and 334, and with the planarity adjustment feature.

In some embodiments, at least one of collection camera 314 and lens 316 can have a mount comprising a threaded mount or a bayonet mount. The threaded mount can comprise, for example, a C-mount or a CS-mount. The bayonet mount can comprise, for example, an F-mount or a K-mount. In some embodiments, collection camera 314 can be mounted to mounting plate 332 using a mounting ring 346 and a retaining ring 348. In some embodiments, mounting plate 332 can be formed from a metal, such as steel, stainless steel, or aluminum. Collection camera 314 can be secured to mounting ring 346. Mounting ring 346 can fit into a groove 350 formed around a periphery of aperture 334. Retaining ring 348 can fasten to mounting plate 332 and can cover at least a portion of groove 350 and a portion of mounting ring 346, thereby retaining mounting ring 346 within groove 350. In some embodiments, retaining ring 348 can be formed from a metal, such as steel, stainless steel, or aluminum. In some embodiments, a concentricity adjustment feature, such as at least one set screw 352, can protrude radially into groove 350 and can press against an outer periphery 354 of mounting ring 346. The concentricity adjustment feature can locate mounting ring 350 in an x-y plane of groove 350. The x-y plane can be illustrated by a coordinate system 356. In some embodiments, at least one setscrew 352 can have a thread pitch between 80 TPI and 100 TPI, inclusive. In some embodiments, at least one setscrew 352 can be a ball-end type. The concentricity adjustment feature in other embodiments can include cams, motorized screws, fluid-containing bags, and/or inclined planes.

A line segment 358 can represent an image plane of optic assembly 322. An arrow 360 can be centered on optic assembly 322 and normal to its image plane 358. A line segment 362 can represent an image plane of multi-element photo detector 324. An arrow 364 can be centered on multi-element photo detector 324 and normal to its image plane 362.

In operation, the planarity adjustment feature, such as at least one set screw 342, can be used to tilt mounting plate 332 such that image plane 362 can become parallel with image plane 322. The planarity adjustment feature can also used to adjust the distance between optic assembly 322 and multi-element photo detector 324.

The concentricity adjustment feature, such as at least one setscrew 352, can translate mounting ring 346 in the x-y plane. Translating mounting ring 346 can adjust arrow 364 concentrically with arrow 360.

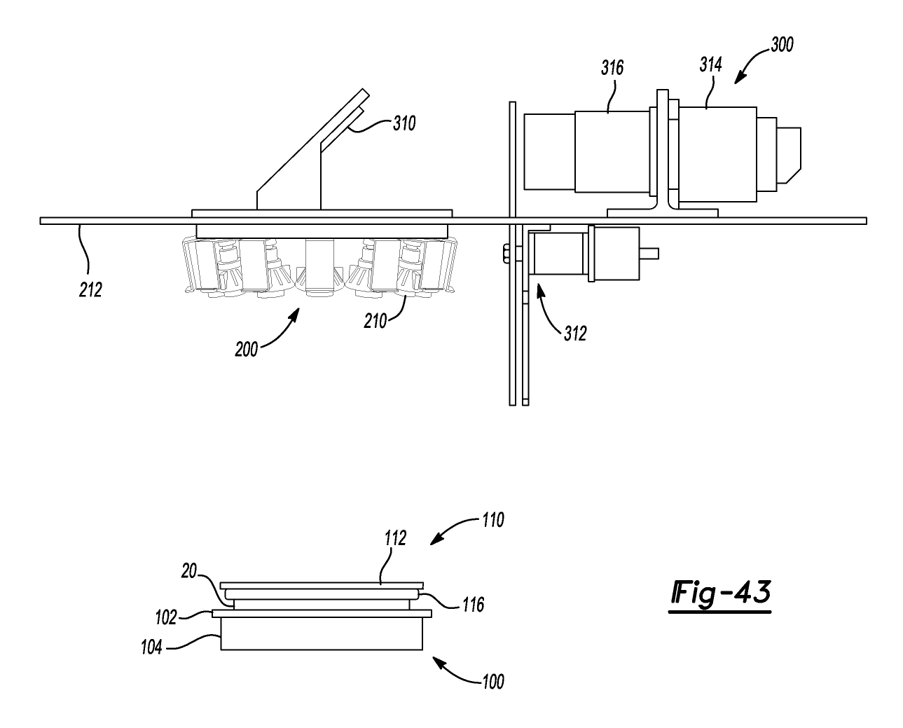

In some embodiments, alignment features 368 can align base plate 328 with support structure 212. Locations of alignment features 368 and dimensions of alignment mount 320 can be selected to place the arrow 360 concentric with a center of microplate 20. Locations of alignment features 356 and dimensions of alignment mount 320 can be selected to place image plane 358 in parallel with an image plane of microplate 20. In some embodiments having collection mirror 310 (of FIGS. 42 and 43), locations of alignment features 356 and dimensions of alignment mount 320 can be selected to place image plane 358 perpendicular with the image plane of microplate 20. In some embodiments, base plate 328 can include a foot plate 366. By way of non-limiting example, alignment features 368 can comprise any combination of dowels and keys.

### Control System

In some embodiments, control system 1010 can be operable to control various portions of high-density sequence detection system 10 and to collect data. In such embodiments, control system 1010 can comprise software and devices operable to collect and analysis data; control operation of electrical, mechanical, and optical portions of high-density sequence detection system 10; and thermocycling. In some embodiments, such data analysis can comprise organizing, manipulating, and reporting of data and derived results to determine relative gene expression within assay 1000, between various test samples, and across multiple test runs.

In some embodiments, control system 1010 can archive data within a database, database retrieval, database analysis and manipulation, and bioinformatics. In some embodiments, control system 1010 can be operable to analyze raw data and among other actions, control operation of high-density sequence detection system 10. Such analysis of raw data can comprise compensating for point spread (PSF), background or base emissions, a unique intensity profile, optical crosstalk, detector and/or optical path variability and noise, misalignment, or movement during operation. This can be accomplished, in some embodiments, by utilizing internal controls in several of the plurality of wells 26, as well as calibrating high-density sequence detection system 10. In some embodiments, data analysis can comprise difference imaging, such as comparing an image from one point in time to an image at a different point in time, or image subtracting. In some embodiments, data analysis can comprise curve fitting based on a specific gene or a gene set. Still further, in some embodiments, data analysis can comprise using no template control (NTC) background or baseline correction. In some embodiments, data analysis can comprise error estimation using confidence values derived in terms of CT. See U.S. Patent Application No. 60/517,506 filed Nov. 4, 2003 and U.S. Patent Application No. 60/519,077 (Attorney Docket No. AB 5043) filed Nov. 10, 2003.

In some embodiments, the present teachings can provide a method for reducing signal noise from an array of pixels of a segmented detector for biological samples. The signal noise comprises a dark current contribution and readout offset contribution. The method can comprise providing a substantially dark condition for the array of pixels, wherein the dark condition comprises being substantially free of fluorescent light emitted from the biological samples, providing a first output signal from a binned portion of the array of pixels by collecting charge for a first exposure duration, transferring the collected charge to an output register and reading out the register, wherein transferring of the collected charge from the binned pixels comprises providing a gate voltage to a region near the binned pixels to move collected charge from the binned pixels, and wherein the collected charge can be transferred in a manner that causes the collected charge to be shifted to the output register, providing a second output signal from each pixel by collecting charge for a second exposure duration, transferring the collected charge to the output register, and reading out the register, providing a third output signal by resetting and reading out the output register, determining the dark current contribution and the readout offset contribution from the first output signal, the second output signal, and the third output signal.

In some embodiments, the present teachings can provide a method of characterizing signal noise associated with operation of a charge-coupled device (CCD) adapted for analysis of biological samples, wherein the signal noise comprises a dark current contribution, readout offset contribution, and spurious change contribution. The method can comprise providing a plurality of first data points associated with first outputs provided from the CCD under a substantially dark condition during a first exposure duration, providing a plurality of second data points associated with second outputs provided from the CCD under the substantially dark condition during a second exposure duration wherein the second duration is different from the first duration, providing a plurality of third data points associated with third outputs provided from a cleared output register of the CCD without comprising charge transferred thereto, determining the dark current contribution per unit exposure time by comparing the first data points and the second data points, determining the readout offset contribution from the third data points, and determining the spurious charge contribution based on the dark current contribution and the readout offset contribution. See U.S. patent application Ser. No. 10/913,601 filed Aug. 5, 2004; U.S. patent application Ser. No. 10/660,460 filed Sep. 11, 2003, and U.S. patent application Ser. No. 10/660,110 filed Sep. 11, 2003.

1. An apparatus for sealing a microplate, said apparatus comprising:
a microplate having a first surface and an opposing second surface;
a plurality of wells formed in said first surface of said microplate, each of said plurality of wells being sized to receive an assay therein; and
a sealing cover disposed over said microplate adjacent said plurality of wells, said sealing cover comprising a plurality of transparent layers, at least one transparent layer of the sealing cover being compliant.

2. The apparatus according to claim 1, further comprising:
a rim portion formed about each of said plurality of wells, each of said rim portions extending upward above said first surface and engageable with said sealing cover to define a sealing interface.

3. The apparatus according to claim 2 wherein said rim portions are made of a first material and said microplate is made of a second material, said first material being different than said second material.

4. The apparatus according to claim 2 wherein said rim portions are made of polystyrene.

5. (canceled)

6. The apparatus according to claim 1 wherein each of said rim portions is plastically deformable and thermally fusible with said sealing cover.

7.-12. (canceled)

13. An apparatus for sealing a microplate, said apparatus comprising:
a microplate having a first surface and an opposing second surface;
a plurality of wells formed in said first surface of said microplate, each of said plurality of wells being sized to receive an assay therein;
a rim portion formed about each of said plurality of wells, each of said rim portions extending upward above said first surface; and
a sealing cover disposed over said microplate adjacent said plurality of wells, said sealing cover comprising a plurality of transparent layers, at least one transparent layer of the sealing cover being engageable with said rim portions to define a sealing interface about each of said plurality of wells.

14. The apparatus according to claim 13 wherein said sealing cover is compliant.

15. The apparatus according to claim 13 wherein said rim portions are made of a first material and said microplate is made of a second material, said first material being different than said second material.

16. The apparatus according to claim 13 wherein said rim portions are made of polystyrene.

17. (canceled)

18. The apparatus according to claim 13 wherein each of said rim portions is plastically deformable and thermally fusible with said sealing cover.

19. An apparatus for sealing a microplate, said apparatus comprising:
a microplate having a first surface and an opposing second surface;
a plurality of wells formed in said first surface of said microplate, each of said plurality of wells being sized to receive an assay therein;
a sealing layer disposed over said microplate adjacent said plurality of wells, said sealing layer being transparent; and
a compliant layer disposed over said sealing cover, said compliant layer being transparent.

20. The apparatus according to claim 19, further comprising:
a rim portion formed about each of said plurality of wells, each of said rim portions extending upward above said first surface and engageable with said sealing layer to define a sealing interface.

21. The apparatus according to claim 20 wherein said rim portions are made of a first material and said microplate is made of a second material, said first material being different than said second material.

22. The apparatus according to claim 20 wherein said rim portions are made of polystyrene.

23. The apparatus according to claim 19 wherein each of said rim portions is plastically deformable and thermally fusible with said sealing layer.

24. The apparatus according to claim 19 wherein the sealing layer comprises an adhesive layer.

25. The apparatus according to claim 1 wherein the sealing cover comprises an adhesive layer.

26. The apparatus according to claim 13 wherein the sealing cover comprises an adhesive layer.
